(19) United States
(12) Patent Application Publication
(10) Pub. No.: US 2024/0075087 A1
(43) Pub. Date: Mar. 7, 2024

(54) COMPOSITIONS AND METHODS COMPRISING HEMP EXTRACT FOR THE TREATMENT OF ANIMALS IN NEED

(71) Applicant: Portland Technology Holdings LLC, South Portland, ME (US)

(72) Inventor: Joseph Wakshlag, South Portland, ME (US)

(21) Appl. No.: 18/363,793

(22) Filed: Aug. 2, 2023

Related U.S. Application Data
(63) Continuation of application No. PCT/US22/70630, filed on Feb. 11, 2022.
(60) Provisional application No. 63/197,179, filed on Jun. 4, 2021.
(60) Provisional application No. 63/148,467, filed on Feb. 11, 2021.

(51) Int. Cl. A61K 36/185; A61K 9/00; A61K 31/00; A61P 1/12; A61P 17/02; A61P 17/04; A61P 25/22; A61P 29/00

(52) U.S. Cl. CPC A61K 36/185; A61K 9/0017; A61K 9/0056; A61K 31/658; A61P 1/12; A61P 17/02; A61P 17/04; A61P 25/22; A61P 29/00

(57) ABSTRACT

This disclosure relates to non-naturally occurring or engineered compositions and methods comprising one or more hemp extracts for the treatment of diseases, disorders, syndromes, and/or conditions in animals in need.

### FIELD OF THE INVENTION

This disclosure relates to non-naturally occurring or engineered compositions and methods comprising one or more hemp extracts for the treatment of diseases, disorders, syndromes, and/or conditions in animals in need.

### BACKGROUND

Industrial hemp products with 0.3% or less of tetrahydrocannabinol (THC) are legal according to the Industrial Hemp Act. These products alone or when combined with other cannabinoids (CBDs) are reported to have health benefits including analgesic, anti-anxiety, anti-inflammatory, anti-anxiolytic, and anti-epileptic. There are numerous companies selling hemp products including CBD oil claiming that they are safe and effective for various medical conditions in humans and animals. In the absence of an optimal treatment using traditional pharmacological agents for patients in need, other potentially efficacious pharmacological agents such as CBDs are often sought. However, there is very little published data to support safety and efficacy claims for the use of these products in human and veterinary patients.

Thus, there is a need for safer and more targeted hemp products.

### SUMMARY

In one aspect, the present technology relates to methods for treating recurrent diarrhea disease in a veterinary subject in need thereof comprising administering to the subject a therapeutically effective amount of hemp extract. In some embodiments, the veterinary subject is a primate. In some embodiments, the primate is a non-human primate. In some embodiments, the non-human primate is a macaque. In some embodiments, the recurrent diarrhea disease is idiopathic diarrhea disease. In some embodiments, the hemp extract is administered orally. In some embodiments, the hemp extract is administered in the form of a chew, a marshmallow, a gummy, or via syringe. In some embodiments, the hemp extract is administered at a dosage of about 2 mg/kg, about 4 mg/kg, or about 8 mg/kg. In some embodiments, the hemp extract is administered until the veterinary subject has a stool score of ≤2 for 3 days or for 21 days. In some embodiments, the hemp extract is administered once a day, twice a day, three times a day, or four times a day.

In another aspect, the present technology relates to methods for treating inflammation in a veterinary subject in need thereof comprising administering to the subject a therapeutically effective amount of hemp extract. In some embodiments, in the veterinary subject is a dog. In some embodiments, the hemp extract comprises CBD, CBDA, or a combination thereof. In some embodiments, the hemp extract modulates neutrophil function, generation of reactive oxygen species, phagocytosis, eicosanoid concentrations, chemotaxis, cytokine production, and/or fibroblast response. In some embodiments, these modulations are measured using in vitro assays using samples from the veterinary subject. In some embodiments, these modulations occur in vivo and are optionally measured in vivo.

In another aspect, the present technology relates to methods for treating noise aversion in a veterinary subject in need thereof comprising administering to the subject a therapeutically effective amount of hemp extract. In some embodiments, the veterinary subject is a dog. In some embodiments, the noise aversion is a phobia. In some embodiments, the phobia is storm phobia or noise phobia. In some embodiments, the noise aversion is to fireworks or thunderstorms. In some embodiments, the hemp extract is administered at a dosage of 2 mg/kg to 10 mg/kg. In some embodiments, the hemp extract is administered at a dosage of 4 mg/kg to 10 mg/kg.

In another aspect, the present technology relates to methods for treating feather plucking in a veterinary subject in need thereof, comprising administering to the subject a therapeutically effective amount of hemp extract. In some embodiments, the veterinary subject is a bird. In some embodiments, the hemp extract comprises CBD, CBDA, or a combination thereof. In some embodiments, the hemp extract comprises 70 mg/mL total cannabinoids. In some embodiments, the hemp extract comprises 60 mg/mL of CBD and CBDA. In some embodiments, the hemp extract is administered at a dosage of 15 mg/kg, twice daily, for three months. In some embodiments, the hemp extract is administered at a dosage from 30 mg/kg to 120 mg/kg, twice daily, for three months. In some embodiments, the hemp extract is administered at a dosage from 40 mg/kg to 80 mg/kg, twice daily, for three months. In some embodiments, the hemp extract is administered at a dosage of 30 mg/kg, twice daily, for three months. In some embodiments, the hemp extract is administered at a dosage of 60 mg/kg, twice daily, for three months. In some embodiments, the hemp extract is administered at a dosage of 120 mg/kg, twice daily, for three months. In another aspect, the present technology relates to methods for treating arthritis in a veterinary subject in need thereof, comprising administering to the subject a therapeutically effective amount of hemp extract. In some embodiments, the veterinary subject is a rabbit. In some embodiments, the hemp extract comprises CBD, CBDA, or a combination thereof. In some embodiments, the hemp extract is administered at a dosage of 10 mg/kg to 25 mg/kg. In some embodiments, the hemp extract is administered at a dosage of about 15 mg/kg. In some embodiments, the hemp extract is administered at a dosage of about 20 mg/kg. In some embodiments, the hemp extract is administered twice daily.

In another aspect, the present technology relates to methods for treating lameness in a veterinary subject in need thereof, comprising administering to the subject a therapeutically effective amount of hemp extract. In some embodiments, the veterinary subject is a horse. In some embodiments, the hemp extract comprises CBD, CBDA, or a combination thereof. In some embodiments, the hemp extract is administered at a dosage of 1 mg/kg every 12 hours. In some embodiments, the hemp extract is administered as an acute dose of 2 mg/kg to 8 mg/kg.

In another aspect, the present technology relates to methods for promoting wound healing in a veterinary subject in need thereof comprising administering to the subject a therapeutically effective amount of hemp extract. In some embodiments, the hemp extract is administered topically. In some embodiments, the veterinary subject is a dog. In some embodiments, the hemp extract comprises CBG and CBGA. In some embodiments, 20 mg of CBG and CBGA is applied to the wound. In some embodiments, the CBG and CBGA are applied to the wound every 12 hours.

In another aspect, the present technology relates to methods for treating cancer in a veterinary subject in need thereof, comprising administering to the subject a therapeutically effective amount of hemp extract. In some embodiments, the veterinary subject is a dog. In some embodiments, the hemp extract comprises CBD, CBDA, or a combination thereof. In some embodiments, the hemp extract is administered at a dose of 5 mg/kg.

In another aspect, the present technology relates to methods for treating pyoderma in a veterinary subject in need thereof, comprising administering to the subject a therapeutically effective amount of hemp extract. In some embodiments, the hemp extract is administered topically. In some embodiments, the veterinary subject is a dog. In some embodiments, the hemp extract comprises CBD and CBG. In some embodiments 30 mg of CBD and CBG are applied to location of the pyoderma. In some embodiments 35 mg of CBD and CBG are applied to location of the pyoderma. In some embodiments 40 mg of CBD and CBG are applied to location of the pyoderma. In some embodiments 45 mg of CBD and CBG are applied to location of the pyoderma. In some embodiments 50 mg of CBD and CBG are applied to location of the pyoderma. In some embodiments 30 mg to 50 mg of CBD and CBG are applied to location of the pyoderma. In some embodiments, the CBD and CBG are applied to location of the pyoderma every 12 hours.

In another aspect, the present technology relates to methods for treating atopy in a veterinary subject in need thereof, comprising administering to the subject a therapeutically effective amount of hemp extract. In some embodiments, the veterinary subject is a dog. In some embodiments, the hemp extract comprises CBD and CBDA. In some embodiments the hemp extract is administered orally. In some embodiments the hemp extract is administered via a capsule. In some embodiments, the hemp extract comprises CBD and CBDA and the hemp extract is administered such that a dose of about 2 mg/kg of CBD and CBDA is administered. In some embodiments, the hemp extract is administered once every 12 hours.

In another aspect, the present technology relates to methods for treating pruritus in a veterinary subject in need thereof, comprising administering to the subject a therapeutically effective amount of hemp extract. In some embodiments, the veterinary subject is a dog. In some embodiments, the hemp extract comprises CBD and CBDA. In some embodiments the hemp extract is administered orally. In some embodiments the hemp extract is administered via a capsule. In some embodiments, the hemp extract comprises CBD and CBDA and the hemp extract is administered such that a dose of about 2 mg/kg of CBD and CBDA is administered. In some embodiments, the hemp extract is administered once every 12 hours.

In another aspect, the present technology relates to methods for treating a neural condition in a subject in need thereof, comprising administering to the subject a therapeutically effective amount of hemp extract. In some embodiments, the subject is a human. In some embodiments, the subject is a veterinary subject. In some embodiments, the neural condition comprises a traumatic neural injury or a degenerative neural disease (e.g., a proteopathy such as ALS, Parkinson's disease, dementia, or Alzheimer's disease). In some embodiments, the hemp extract comprises tetrahydrocannabinolic acid (THCA).

In some embodiments, the hemp extract comprises cannabidiol and cannabidiolic acid, wherein the ratio of cannabidiol to cannabidiolic acid is about 0.6:1 to about 1:0.6. In some embodiments, the hemp extract further comprises cannabigerolic acid, Δ9-tetrahydrocannabinol, and cannabichromene. In some embodiments, the hemp extract also comprises THCA.

In some embodiments, the hemp extract comprises:
cannabidiol;
cannabidiolic acid;
cannabigerolic acid;
Δ9-tetrahydrocannabinol; and
cannabichromene;
wherein the ratio of cannabidiol to cannabidiolic acid is about 0.6:1 to about 1:0.6.

In some embodiments, the hemp extract further comprises:
α-pinene;
β-myrcene;
β-pinene;
δ-limonene;
linalool;
β-caryophyllene;
α-humulene;
nerolidol 2;
guaiol;
caryophyllene oxide; and
α-bisabolol.

In some embodiments, the concentration of Δ9-tetrahydrocannabinol is insufficient to produce a psychotropic effect. In some embodiments, the ratio of Δ9-tetrahydrocannabinol to the other cannabinoids is about 1:25. In some embodiments, the concentration of Δ9-tetrahydrocannabinol is less than about 1 mg/mL. In some embodiments, the concentration of Δ9-tetrahydrocannabinol is less than about 0.5 mg/mL. In some embodiments, the concentration of Δ9-tetrahydrocannabinol is less than about 0.3 mg/mL. In some embodiments, the concentration of Δ9-tetrahydrocannabinol is less than about 0.2 mg/mL. In some embodiments, the concentration of Δ9-tetrahydrocannabinol is less than about 0.1 mg/mL. In some embodiments, the concentration of Δ9-tetrahydrocannabinol is about 0 mg/mL. In some embodiments, the hemp extract also comprises THCA.

In some embodiments, the hemp extract comprises:
about 1-10 mg/mL of cannabidiol;
about 1-10 mg/mL of cannabidiolic acid;
about 0.05-0.2 mg/mL cannabigerolic acid;
about 0.1-0.3 mg/mL Δ9-tetrahydrocannabinol; and
about 0.1-0.4 mg/mL cannabichromene.

In some embodiments, the hemp extract comprises:
about 5 mg/mL of cannabidiol;
about 5 mg/mL of cannabidiolic acid;
about 0.11 mg/mL cannabigerolic acid;
about 0.25 mg/mL Δ9-tetrahydrocannabinol; and
about 0.27 mg/mL cannabichromene.

In some embodiments, the hemp extract comprises:
about 0.09-0.13% α-pinene;
about 0.23-0.44% β-myrcene;
about 0.04-0.09% β-pinene;
about 0.05-0.09% δ-limonene;
about 0.03-0.06% linalool;
about 0.04-0.07% β-caryophyllene;
about 0.02-0.04% α-humulene;
about .04-0.07% nerolidol 2;
about 0.04-0.08% caryophyllene oxide; and
about 0.01-0.04% α-bisabolol.

In some embodiments, the hemp extract further comprises:
camphene;
β-ocimene;
eucalyptol;
isopulegol; and/or
nerolidol 1.

In some embodiments, the hemp extract also comprises THCA.

In some embodiments, the hemp extract comprises:
about 0.02% camphene;
about 0.02-0.03% β-ocimene;
about 0.02-0.05% eucalyptol;
about 0.02% isopulegol; and/or
about 0.02-0.04% nerolidol 1.

In some embodiments, the hemp extract also comprises THCA.

In some embodiments, the hemp extract is formulated in a carrier. In some embodiments, the carrier is selected from the group consisting of hemp seed oil, linseed oil, olive oil, fish oil, salmon oil, coconut oil, catnip oil, sesame oil, MCT oil, and grapeseed oil. In some embodiments, the carrier is grapeseed oil. In some embodiments, the carrier is catnip oil. In some embodiments, the carrier is sesame oil. In some embodiments, the hemp extract comprises lecithin. In some embodiments, the lecithin is sunflower lecithin. In some embodiments, the sunflower lecithin is up to 40%. In some embodiments, the hemp extract further comprises NF-971P. In some embodiments, the NF-971P is up to 2% weight/volume ratio. In some embodiments, the hemp extract comprises nepetalactone. In some embodiments, the hemp extract comprises taurine.

In some embodiments, the hemp extract comprises:
cannabidiol;
cannabidiolic acid;
cannabigerolic acid;
Δ9-tetrahydrocannabinol; and
cannabichromene;
wherein the carrier is grapeseed oil.

In some embodiments, the hemp extract comprises:
cannabidiol;
cannabidiolic acid;
cannabigerolic acid;
cannabigerol;
Δ9-tetrahydrocannabinol; and
cannabichromene;
wherein the carrier is grapeseed oil.

In some embodiments, the ratio of cannabidiol to cannabidiolic acid is selected from the group consisting of about 1:100, about 1:50, about 1:10, and about 1:1. In some embodiments, the ratio of cannabidiol to cannabidiolic acid is about 1:1. In some embodiments, the concentration of Δ9-tetrahydrocannabinol is insufficient to produce a psychotropic effect. In some embodiments, the ratio of Δ9-tetrahydrocannabinol to the other cannabinoids is about 1:25. In some embodiments, the concentration of Δ9-tetrahydrocannabinol is less than about 1 mg/mL. In some embodiments, the concentration of Δ9-tetrahydrocannabinol is less than about 0.5 mg/mL. In some embodiments, the concentration of Δ9-tetrahydrocannabinol is less than about 0.3 mg/mL. In some embodiments, the hemp extract also comprises THCA.

In some embodiments, the hemp extract comprises:
about 1-10 mg/mL of cannabidiol;
about 1-10 mg/mL of cannabidiolic acid;
about 0.05-0.2 mg/mL cannabigerolic acid;
about 0.1-0.3 mg/mL Δ9-tetrahydrocannabinol; and
about 0.1-0.4 mg/mL cannabichromene.

In some embodiments, the hemp extract comprises:
about 5 mg/mL of cannabidiol;
about 5 mg/mL of cannabidiolic acid;
about 0.11 mg/mL cannabigerolic acid;
about 0.25 mg/mL Δ9-tetrahydrocannabinol; and
about 0.27 mg/mL cannabichromene.

In some embodiments, the hemp extract comprises:
about 1-10 mg/mL of cannabidiol;
about 1-10 mg/mL of cannabidiolic acid;
about 0.05-0.2 mg/mL cannabigerolic acid;
cannabigerol;
about 0.1-0.3 mg/mL Δ9-tetrahydrocannabinol; and
about 0.1-0.4 mg/mL cannabichromene.

In some embodiments, the hemp extract comprises:
about 5 mg/mL of cannabidiol;
about 5 mg/mL of cannabidiolic acid;
cannabigerol;
about 0.11 mg/mL cannabigerolic acid;
about 0.25 mg/mL Δ9-tetrahydrocannabinol; and
about 0.27 mg/mL cannabichromene.

In some embodiments, the hemp extract comprises:
α-pinene;
β-myrcene;
β-pinene;
δ-limonene;
linalool;
β-caryophyllene;
α-humulene;
nerolidol 2;
guaiol;
caryophyllene oxide; and
α-bisabolol.

In some embodiments, the hemp extract comprises:
about 0.09-0.13% α-pinene;
about 0.23-0.44% β-myrcene;
about 0.04-0.09% β-pinene;
about 0.05-0.09% δ-limonene;
about 0.03-0.06% linalool;
about 0.04-0.07% β-caryophyllene;
about 0.02-0.04% α-humulene;
about 0.04-0.07% nerolidol 2;
about 0.02-0.04% guaiol;
about 0.04-0.08% caryophyllene oxide; and
about 0.01-0.04% α-bisabolol.

In some embodiments, the hemp extract further comprises:
camphene;
β-ocimene;
eucalyptol;
isopulegol; and/or
nerolidol 1

In some embodiments, the hemp extract comprises:
about 0.02% camphene;
about 0.02-0.03% β-ocimene;
about 0.02-0.05% eucalyptol;
about 0.02% isopulegol; and/or
about 0.02-0.04% nerolidol 1.

In some embodiments, the hemp extract is administered in a dosage form comprising one or more pharmaceutically acceptable additives, flavoring agents, surfactants, and adjuvants. In some embodiments, the flavoring agent is selected from the group consisting of peppermint oil, mango extract, beef, poultry, and seafood. In some embodiments, the flavoring agent is selected from the group consisting of peanut butter, catnip oil, chicken liver powder, poultry extract, maltodextrin, butter, and bacon. In some embodiments, the flavoring agent is chicken liver powder. In some embodiments, the flavoring agent is catnip oil. In some embodiments, the flavoring agent is molasses or dry molasses. In some embodiments, the flavoring agent is peanut butter. In some embodiments, the dosage form comprises nepetalactone. In some embodiments, the dosage form comprises taurine. In some embodiments, the dosage form is formulated as a sublingual spray. In some embodiments, the dosage form is formulated as a water, polyethylene glycol, glycerol, or alcohol soluble solution or cream for topical or transdermal application. In some embodiments, the dosage form is formulated as a gel for buccal or mucosal administration. In some embodiments, the dosage form is formulated as a paste for buccal or mucosal administration. In some embodiments, the dosage form is formulated as a powder. In some embodiments, the dosage form is formulated as a solution for subcutaneous injection. In some embodiments, the dosage form is formulated as a tablet. In some embodiments, the dosage form is formulated as a capsule. In some embodiments, the dosage form is formulated as a hard chewable. In some embodiments, the dosage form is formulated as a soft chewable. In some embodiments, the dosage form is formulated for administration using a nebulizer. In some embodiments, the dosage form is formulated for inhalation. In some embodiments, the dosage form is formulated for administration using a pet collar. In some embodiments, the composition is formulated as an edible product for oral administration. In some embodiments, the edible product comprises pet food.

In some embodiments, the dosage form is formulated as a chew for oral administration. In some embodiments, the chew is produced using cold extrusion. In some embodiments, the weight of the chew is about 0.5-10 g. In some embodiments, the weight of the chew is about 4 g, about 6 g, about 9 g, or about 10 g. In some embodiments, the weight of the chew is about 4 g.

In some embodiments, the chew comprises:
about 7 mg of cannabidiol;
about 6 mg of cannabidiolic acid;
about 0.12 mg cannabigerolic acid;
about 0.32 mg Δ9-tetrahydrocannabinol; and
about 0.36 mg cannabichromene.

In some embodiments, chew also comprises THCA.

In some embodiments, the dosage form is formulated in a carrier for oral administration. In some embodiments, the carrier is selected from the group consisting of hemp seed oil, linseed oil, olive oil, fish oil, salmon oil, coconut oil, catnip oil, sesame oil, MCT oil, and grapeseed oil. In some embodiments, the carrier is grapeseed oil. In some embodiments, the carrier is catnip oil. In some embodiments, the carrier is sesame oil.

In some embodiments, the dosage form comprises:
glucosamine HCl;
chondroitin Sulfate (76%);
brewer's yeast;
arabic gum;
guar gum;
a flavoring agent;
Verdilox;
Previon;
hemp extract;
glycerin;
sunflower lecithin; and
water.

In some embodiments, the dosage form comprises:
about 12-17% glucosamine HCl;
about 1-4% chondroitin sulfate (76%);
about 29-33% brewer's yeast;
about 3-6% arabic gum;
about 0.5-2% guar gum;
about 12-16% of a flavoring agent;
about 0.01-0.1% Verdilox;
about 0.5-1.5% Previon;
about 3-6% hemp extract;
about 13-17% glycerin;
about 3-7% sunflower lecithin; and
about 3-7% water.

In some embodiments, the dosage form comprises:
about 15.6% glucosamine HCl;
about 2.6% chondroitin sulfate (76%);
about 30% brewer's yeast;
about 4.7% arabic gum;
about 0.9% guar gum;
about 14.2% of a flavoring agent;
about 0.05% Verdilox;
about 0.9% Previon;
about 4.7% hemp extract;
about 15.1% glycerin;
about 5.7% sunflower lecithin; and
about 5.7% water.

In some embodiments, the dosage form comprises:
glucosamine HCl;
hyaluronic acid;
brewer's yeast;
arabic gum;
guar gum;
a flavoring agent;
Verdilox;
Previon;
hemp extract;
glycerin;
sunflower lecithin; and
water.

In some embodiments, the dosage form comprises:
about 12-17% glucosamine HCl;
about 0.01-1% hyaluronic acid;
about 29-33% brewer's yeast;
about 3-6% arabic gum;
about 0.5-2% guar gum;
about 12-16% of a flavoring agent;
about 0.01-0.1% Verdilox;
about 0.5-1.5% Previon;
about 3-6% hemp extract;
about 13-17% glycerin;
about 3-7% sunflower lecithin; and
about 3-7% water.

In some embodiments, the dosage form comprises:
about 16% glucosamine HCl;
about 0.1% hyaluronic acid;
about 30.6% brewer's yeast;
about 4.8% arabic gum;
about 0.97% guar gum;
about 14.5% of a flavoring agent;
about 0.05% Verdilox;
about 0.97% Previon;
about 4.8% hemp extract;
about 15.5% glycerin;
about 5.8% sunflower lecithin; and
about 5.8% water.

In some embodiments, the dosage form comprises:
hemp extract;
peanut butter;
rice bran;
glucosamine HCl;
sweet potato;
dry molasses;
sorbic acid;
brewer's yeast;
sugar;
water;
glycerin;
potato starch;
dehydrated peanut butter;
rice starch; and
guar gum.

In some embodiments, the dosage form comprises:
about 3.0-10.0% hemp extract;
about 10.0-20.0% peanut butter;
about 10.0-15.0% rice bran;
about 5.0-15.0% glucosamine HCl;
about 4.0-10.0% sweet potato;
about 6.0-13.0% dry molasses;
about 0.5-5.0% sorbic acid;
about 2.0-8.0% brewer's yeast;
about 3.0-8.0% sugar;
about 5.0-15.0% water;
about 8.0-18.0% glycerin;
about 1.0-8.0% potato starch;
about 0.5-5.0% dehydrated peanut butter;
about 1.0-5.0% rice starch; and
about 1.0-5.0% guar gum.

In some embodiments, the dosage form comprises:
about 5.0% hemp extract;
about 15.0% peanut butter;
about 12.5% rice bran;
about 12.75% glucosamine HCl;
about 5.5% sweet potato;
about 8.0% dry molasses;
about 1% sorbic acid;
about 5.0% brewer's yeast;
about 6.0% sugar;
about 9.25% water;
about 13.0% glycerin;
about 2.0% potato starch;
about 1.0% dehydrated peanut butter;
about 2.0% rice starch; and
about 2.0% guar gum.

In some embodiments, the dosage form comprises:
about 5.0% hemp extract;
about 15.0% peanut butter;
about 13.0% rice bran;
about 8.5% glucosamine HCl;
about 6.0% sweet potato;
about 9.0% dry molasses;
about 1% sorbic acid;
about 5.0% brewer's yeast;
about 6.0% sugar;
about 9.5% water;
about 13.0% glycerin;
about 4.0% potato starch;
about 1.0% dehydrated peanut butter;
about 2.0% rice starch; and
about 2.0% guar gum.

In some embodiments, the dosage form comprises:
hemp extract;
peanut butter;
rice bran;
glucosamine HCl;
sweet potato;
dry molasses;
sorbic acid;
brewer's yeast;
sugar;
water;
glycerin;
potato starch;
dehydrated peanut butter;
DigestaWell PET;
rice starch; and
guar gum.

In some embodiments, the dosage form comprises:
about 3.0-10.0% hemp extract;
about 5.0-20.0% peanut butter;
about 10.0-15.0% rice bran;
about 5.0-15.0% glucosamine HCl;
about 4.0-10.0% sweet potato;
about 6.0-13.0% dry molasses;
about 0.5-5.0% sorbic acid;
about 2.0-8.0% brewer's yeast;
about 3.0-8.0% sugar;
about 5.0-15.0% water;
about 8.0-18.0% glycerin;
about 1.0-8.0% potato starch;
about 0.5-5.0% dehydrated peanut butter;
about 0.1-3.0% DigestaWell PET;
about 1.0-8.0% rice starch; and
about 1.0-5.0% guar gum

In some embodiments, the dosage form comprises:
about 5.0% hemp extract;
about 10.0% peanut butter;
about 12.0% rice bran;
about 12.75% glucosamine HCl;
about 5.5% sweet potato;
about 8.0% dry molasses;
about 1% sorbic acid;
about 5.0% brewer's yeast;
about 6.0% sugar;
about 7.25% water;
about 10.0% glycerin;
about 5.0% potato starch;
about 4.0% dehydrated peanut butter;
about 0.5% DigestaWell PET;
about 6.0% rice starch; and
about 2.0% guar gum.

In some embodiments, the dosage form comprises:
about 5.0% hemp extract;
about 10.0% peanut butter;
about 12.5% rice bran;
about 8.5% glucosamine HCl;
about 8.0% sweet potato;
about 9.0% dry molasses;
about 1% sorbic acid;
about 5.0% brewer's yeast;
about 6.0% sugar;
about 6.0% water;
about 10.0% glycerin;
about 6.0% potato starch;
about 4.0% dehydrated peanut butter;
about 0.5% DigestaWell PET;
about 6.5% rice starch; and
about 2.0% guar gum.

In some embodiments, the dosage form comprises:
hemp extract;
peanut butter;
rice bran;
glucosamine HCl;
sweet potato;
dry molasses;
sorbic acid;
brewer's yeast;
sugar;
water;
glycerin;
potato starch;
dehydrated peanut butter;
chondroitin;
DigestaWell PET;
rice starch; and
guar gum.

In some embodiments, the dosage form comprises:
about 3.0-10.0% hemp extract;
about 5.0-20.0% peanut butter;
about 10.0-15.0% rice bran;
about 5.0-15.0% glucosamine HCl;
about 4.0-10.0% sweet potato;
about 6.0-13.0% dry molasses;
about 0.5-5.0% sorbic acid;
about 2.0-8.0% brewer's yeast;
about 3.0-8.0% sugar;
about 5.0-15.0% water;
about 8.0-18.0% glycerin;
about 1.0-8.0% potato starch;
about 0.5-5.0% dehydrated peanut butter;
about 0.5-5.0% chondroitin;
about 0.1-3.0% DigestaWell PET;
about 1.0-8.0% rice starch; and
about 1.0-5.0% guar gum.

In some embodiments, the dosage form comprises:
about 5.0% hemp extract;
about 10.0% peanut butter;
about 12.0% rice bran;
about 12.75% glucosamine HCl;
about 5.5% sweet potato;
about 8.0% dry molasses;
about 1% sorbic acid;
about 5.0% brewer's yeast;
about 6.0% sugar;
about 7.25% water;
about 10.0% glycerin;
about 4.0% potato starch;
about 4.0% dehydrated peanut butter;
about 2.5% chondroitin;
about 0.5% DigestaWell PET;
about 4.5% rice starch; and
about 2.0% guar gum.

In some embodiments, the dosage form comprises:
about 5.0% hemp extract;
about 10.0% peanut butter;
about 12.5% rice bran;
about 8.5% glucosamine HCl;
about 8.0% sweet potato;
about 9.0% dry molasses;
about 1% sorbic acid;
about 5.0% brewer's yeast;
about 6.0% sugar;
about 6.0% water;
about 10.0% glycerin;
about 5.0% potato starch;
about 4.0% dehydrated peanut butter;
about 2.5% chondroitin;
about 0.5% DigestaWell PET;
about 5.0% rice starch; and
about 2.0% guar gum.

In some embodiments, the hemp extract, dosage form, or pharmaceutical composition is packaged to provide one or more doses of hemp extract per package. In some embodiments, the package is resealable. In some embodiments, one dose of hemp extract is a therapeutically effective amount.

### BRIEF DESCRIPTION OF THE DRAWINGS

Aspects, features, benefits, and advantages of the embodiments described herein will be apparent with regard to the following description, appended claims, and accompanying drawings where:

FIG. 1A is a graph showing cannabinoid treated 48-hour MTT proliferation assays performed with cannabidiol (CBD) according to embodiments of the present teachings;

FIG. 1B a graph showing cannabinoid treated 48-hour MTT proliferation assays performed with cannabidiolic acid (CBDA) according to embodiments of the present teachings;

FIG. 1C a graph showing cannabinoid treated 48-hour MTT proliferation assays performed with CBD-rich whole plant hemp extract according to embodiments of the present teachings;

FIG. 2 is a bar chart showing the percentage of trypan blue positive for fibroblast, 17-71, D17 and CMT12 at 48 after treatment with CBD at 15, 7.5, 3.75 ug/mL and with methanol vehicle control (VC) according to embodiments of the present teachings;

FIG. 3A are images of immunoblottings for cleaved caspase 3 (17 Kda) after 8 and 16 hours of CBD treatment as compared with methanol vehicle control according to embodiments of the present teachings;

FIG. 3B is a bar chart showing the apoptosis of neoplastic cell line 17-71 after treatment with 15 μg/mL of CBD according to embodiments of the present teachings;

FIG. 3C is a bar chart showing the apoptosis of neoplastic cell line D17 after treatment with 15 μg/mL of CBD according to embodiments of the present teachings;

FIG. 3D is a bar chart showing the apoptosis of neoplastic cell line CMT12 after treatment with 15 μg/mL of CBD according to embodiments of the present teachings;

FIG. 4A are images of time-course immunoblottings for phosphorylated MAP kinases in relationship to baseline protein expression with vehicle control treated or 10 μg/mL CBD for ERK and phosphorylated ERK expressions at time 2, 4 and 8 hours according to embodiments of the present teachings;

FIG. 4B are images of time-course immunoblottings for phosphorylated MAP kinases in relationship to baseline protein expression with vehicle control treated or 10 μg/mL CBD for JNK and phosphorylated JNK expressions at time 2, 4 and 8 hours according to embodiments of the present teachings;

FIG. 5A are images of immunoblotting for LC3 I/II in 17-71, CMT12 and D17 cell lines treated for 2, 4 or 8 hours with vehicle control or 10 μg/mL CBD according to embodiments of the present teachings; and

FIG. 5B are immunofluorescence images for LC3A/B with CMT12 (600×) and D17 (400×) cells depicted in columns according to embodiments of the present teachings.

FIG. 6A is a box and whisker plot showing VAS scores for dogs in the CBD treatment group and control group at zero, 2, and 4 weeks. Significant differences are marked with asterisks at zero and 4 weeks.

FIG. 6B is a box and whisker plot showing CADESI scores for dogs in the CBD treatment group and control group at zero, 2, and 4 weeks. Significant differences are marked with asterisks at zero and 4 weeks.

FIG. 7A is a graph showing alkaline aminotransferase levels in dogs treated with either the CBD treatment or the placebo. Values are shown for both zero and 4 weeks.

FIG. 7B is a graph showing alkaline phosphatase levels in dogs treated with either the CBD treatment or the placebo. Values are shown for both zero and 4 weeks.

FIG. 8 is a bar plot showing alkaline phosphatase levels in dogs when the CBD treatment was combined with various other drugs, as indicated, or when the CBD treatment was administered without other drugs (right-most column).

FIG. 9A is a plot showing serum levels of monocyte chemoattractant protein-1 (MCP-1) for dogs receiving either the CBD treatment or placebo at zero and 4 weeks.

FIG. 9B is a plot showing serum levels of IL-6 for dogs receiving either the CBD treatment or placebo at zero and 4 weeks.

FIG. 10A is a plot showing serum levels of IL-31 for dogs receiving either the CBD treatment or placebo at zero and 4 weeks.

FIG. 10B is a plot showing serum levels of IL-34 for dogs receiving either the CBD treatment or placebo at zero and 4 weeks.

FIG. 11 shows serum levels of CBDA, CBD, THCA, and THC, at the indicated time points in macaques that received an oral hemp extract dose.

FIG. 12A shows CYP3A12 activity after treatment with vehicle or various potential inhibitors. Data is shown for each potential inhibitor both without preincubation (left bars, no outline) and with a 20 min preincubation (right bars, with outline).

FIG. 12B shows CYP2D15 activity after treatment with vehicle or various potential inhibitors. Data is shown for each potential inhibitor both without preincubation (left bars, no outline) and with a 20 min preincubation (right bars, with outline).

FIG. 12C shows CYP2B11 activity after treatment with vehicle or various potential inhibitors. Data is shown for each potential inhibitor both without preincubation (left bars, no outline) and with a 20 min preincubation (right bars, with outline).

FIG. 13A shows CYP3A12 activity after treatment with vehicle or various potential inhibitors. Data is shown for each potential inhibitor at various concentrations. No preincubation was employed.

FIG. 13B shows CYP3A12 activity after treatment with vehicle or various potential inhibitors. Data is shown for each potential inhibitor at various concentrations. A 20 minute preincubation was employed.

FIG. 14A shows CYP2B11 activity after treatment with vehicle or various potential inhibitors. Data is shown for each potential inhibitor at various concentrations. No preincubation was employed.

FIG. 14B shows CYP2B11 activity after treatment with vehicle or various potential inhibitors. Data is shown for each potential inhibitor at various concentrations. A 20 minute preincubation was employed.

FIG. 15A shows CYP2D15 activity after treatment with vehicle or various potential inhibitors. Data is shown for each potential inhibitor at various concentrations. No preincubation was employed.

FIG. 15B shows CYP2D15 activity after treatment with vehicle or various potential inhibitors. Data is shown for each potential inhibitor at various concentrations. A 20 minute preincubation was employed.

### DETAILED DESCRIPTION

It will be appreciated that for clarity, the following disclosure will describe various aspects of embodiments. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±5% from the specified value, as such variations are appropriate to perform the disclosed methods.

As used in the specification and in the claims, the term “comprising” may include the embodiments “consisting of” and “consisting essentially of” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “treatment” or “treating,” is defined as the application or administration of a therapeutic agent, i.e., a compound provided herein (alone or in combination with another pharmaceutical agent), to a patient or subject, or application or administration of a therapeutic agent to an isolated tissue or cell line from a patient (e.g., for diagnosis or ex vivo applications), with the purpose to cure, heal, alleviate, relieve, alter, remedy, ameliorate, improve or affect the symptoms of a disease, disorder, syndrome, or condition. Such treatments can be specifically tailored or modified, based on knowledge obtained from the field of pharmacology.

In certain embodiments, the compositions described herein treat and/or reduce the severity of a disease, disorder, syndrome, or condition in a subject. For example, the compositions described herein treat and/or improve one or more symptoms of inflammation, periuria, anxiety, depression, insomnia, pain (e.g., chronic pain, non-chronic pain, neuropathic pain, neurological dysfunction pain, nociceptive pain, post-operation pain), skin disorders, cancer, psychotic disorders, seizure, epilepsy, osteoarthritis, lymphoma, atopy, allergies, diarrhea (e.g., idiopathic diarrhea), noise aversion, feather plucking, hair pulling, skin wounds, pyoderma, gastrointestinal conditions, behavioral issues, obsessive behaviors, migraines, headaches, insect bites, diabetes, inflammatory bowel disease, dermatological conditions (e.g., pruritus, pyoderma), urinary conditions, anxiety, or frustration

As used herein, the term “prevent” or “prevention” means no disorder or disease development if none had occurred, or no further disorder or disease development if there had already been development of the disorder or disease. Also considered is the ability of one to prevent some or all of the symptoms associated with a disorder, disease, and/or condition.

As used herein, the term “use” includes any one or more of the following embodiments of the invention, respectively: the use in the treatment of a disease, disorder, syndrome, and/or condition for the manufacture of pharmaceutical compositions for use in the treatment of this disease, disorder, syndrome, and/or condition, e.g., in the manufacture and/or preparation of a medicament; methods of use of compounds of the invention in the treatment of this disease, disorder, syndrome, and/or condition; pharmaceutical preparations having compounds of the invention for the treatment of this disease, disorder, syndrome, and/or condition; and compounds of the invention for use in the treatment of this disease, disorder, syndrome, and/or condition; as appropriate and expedient, if not stated otherwise.

As used herein, the term “patient,” “individual,” or “subject” is intended to include organisms, which are capable of suffering from or afflicted with a disease, disorder, syndrome, and/or condition. Examples of subjects include animals. Examples of subjects include mammals. Examples of subjects include dogs, cats, horses, cows, pigs, sheep, goats, mice, rabbits, rats, birds, fishes, non-human primates, amphibians, reptiles, and transgenic non-human animals.

When used with respect to methods of treatment/prevention and the use of the compounds and pharmaceutical compositions thereof described herein, an individual “in need thereof” may be an individual who has been diagnosed with or previously treated for the condition to be treated. With respect to prevention, the individual in need thereof may also be an individual who is at risk for a condition (e.g., a family history of the disease, disorder, syndrome, and/or condition, life-style factors indicative of risk for the disease, disorder, syndrome, and/or condition, etc.). Typically, when a step of administering a compound of the invention is disclosed herein, the invention further contemplates a step of identifying an individual or subject in need of the particular treatment to be administered or having the particular condition to be treated.

In some embodiments, the individual is a mammal, including, but not limited to, bovine, equine, feline, rabbit, canine, rodent, or primate. In some embodiments, the mammal is a primate. In some embodiments, the individual is a human or other animal and is elderly, adult, a young adult, a child, an adolescent, neonatal, preweaning, an infant, or a premature infant. In some embodiments, the individual is a non-mammal. In some embodiments, the primate is a non-human primate such as chimpanzees and other apes and monkey species. The term “individual” does not denote a particular age or sex. In some embodiments, the individual is a female. In some embodiments, the individual is a male.

As used herein, the term “pharmaceutically acceptable” refers to a material, such as a carrier or diluent, which does not abrogate the biological activity or properties of the compound, and is relatively non-toxic, i.e., the material can be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with an amount (e.g., a stoichiometric amount) of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” or “carrier” means a pharmaceutically acceptable material, composition or carrier, such as a liquid or solid (e.g., a solid filler), stabilizer, dispersing agent, suspending agent, diluent, excipient, thickening agent, solvent or encapsulating material, involved in carrying or transporting a compound useful within the invention within or to the patient or subject such that it can perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient or subject. Some examples of materials that can serve as pharmaceutically acceptable carriers include: sugars, such as lactose, glucose, sucrose, and the like; starches, such as corn starch potato starch, and the like; cellulose, and its derivatives, such as sodium carboxymethyl cellulose, ethyl cellulose, cellulose acetate, and the like; powdered tragacanth; malt; gelatin; talc; excipients, such as cocoa butter, coconut butter, suppository waxes, and the like; oils, such as peanut oil, cottonseed oil, safflower oil, sesame oil, olive oil, corn oil, soybean oil, and the like; glycols, such as propylene glycol and the like; polyols, such as glycerin, sorbitol, mannitol, polyethylene glycol, and the like; esters, such as ethyl oleate, ethyl laurate, and the like; agar; buffering agents, such as magnesium hydroxide, aluminum hydroxide, and the like; surface active agents; alginic acid; water, such as tap water, purified water, distilled water, milli-q water, pyrogen-free water, and the like; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial agents, antifungal agents, antioxidant agents, absorption delaying agents, preservative agents, stabilizing agents, and the like that are compatible with the activity of the compound useful within the invention, and are physiologically acceptable to the patient or subject. One or more supplementary active and/or inactive compounds can also be incorporated into the compositions. The “pharmaceutically acceptable carrier” or “carrier” can further include one or more pharmaceutically acceptable salts of the one or more compounds useful within the invention. Other additional ingredients that can be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

The term “stabilizer,” as used herein, refers to polymers capable of chemically inhibiting or preventing degradation. Stabilizers are added to formulations of compounds to improve chemical and physical stability of the compound.

As used herein, the term “adjuvant” may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as, for example, citric acid, sorbitan monolaurate, triethanolamine oleate, butylated hydroxytoluene, and the like.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, and/or causes of a disease, or any other desired alteration of a biological system. An appropriate therapeutic amount in any individual case may be determined by one of ordinary skill in the art using routine experimentation.

As used herein, the term “weight percent” is meant to refer to the quantity by weight of a compound and/or component in a composition as the quantity by weight of a constituent component of the composition as a percentage of the weight of the total composition. The weight percent can also be calculated by multiplying the mass fraction by 100. The “mass fraction” is the ratio of one substance of a mass m1 to the mass of the total composition mT such that weight percent=(m1/mT)*100.

“Aqueous buffer” refers to a water solution which resists change in hydronium ion and the hydroxide ion concentration (and consequent pH) upon addition of small amounts of acid or base, or upon dilution. Buffer solutions consist of a weak acid and its conjugate base (more common) and/or a weak base and its conjugate acid (less common). The buffer can be prepared by methods well known in the art with the appropriate buffering agents to give the desired pH value. Examples of the suitable buffering agents include hydrochloric acid, lactic acid, acetic acid, citric acid, malic acid, maleic acid, pyruvic acid, succinic acid, tris-hydroxymethylaminomethane, sodium hydroxide, sodium bicarbonate, phosphoric acid, sodium phosphate, and other biologically acceptable buffering agents. Aqueous buffers are readily available commercially and they can be used in preparation of the compositions of this invention without further treatment.

As used herein, the term “hemp extract” refers to a composition of cannabinoids and terpenes that are isolated from a hemp plant. The terms “hemp extract,” “CBD oil,” and “hemp oil” have the same meaning and are used interchangeably herein. The hemp extract can be obtained by any method known in the art. For example, the hemp extract can be obtained by supercritical (or subcritical) CO₂ extraction, which uses carbon dioxide under high pressure and low temperatures to isolate, preserve and maintain the purity of hemp extract. In an embodiment, the hemp extract is obtained from a supercritical CO₂ extraction. For example, supercritical CO₂ extraction may be performed as described in U.S. Pat. No. 8,895,078, which is incorporated herein by reference in its entirety. Alternatively, a solvent such as petroleum ether, ethanol, methanol, butanol, acetone, dry ice, or olive oil can be used, at room temperature (ambient temperature) with stirring, by passive extraction, heated to a temperature above room temperature, or under reflux, as known in the art to provide the hemp extract. In another embodiment, hemp extract from a butanol extraction is employed as starting material for methods disclosed herein.

As used herein, the term “quality of life”, or “QoL” is generally considered a multidimensional concept that involves subjective evaluation of factors that contribute to overall well-being with a more recent publication suggesting a malaise, anxiety and digestive function. Likert scaling system appears to be a sound assessment of QoL and is used in some embodiments herein (see Giuffrida et al. (2018) J Arner Vet Med Assoc. 252:1073-1083, which is incorporated herein by reference in its entirety). The Pruritus Visual Analog Scale (pVAS) appears to be a sound assessment of QoL and is used in some embodiments herein.

Cannabinoids are compounds isolated from hemp plants. Classes of cannabinoids include for example, without limitation, cannabichromenes, cannabicyclols, cannabidiols, cannabielsoins, cannabigerols, cannabinols, cannabinodiols, cannabitriols, delta-8-tetrahydrocannabinols, and delta-9-tetrahydrocannabinols. Cannabinoid compounds include for example, without limitation, cannabichromene (CBC), cannabichromenic acid (CBCA), cannabichromevarin (CBCV), cannabichromevarinic acid (CBCVA), cannabicyclol (CBL), cannabicyclolic acid (CBLA), cannabicyclovarin (CBLV), cannabidiol (CBD), cannabidiol monomethylether (CBDM), cannabidiolic acid (CBDA), cannabidiorcol (CBD-C1), cannabidivarin (CBDV), cannabidivarinic acid (CBDVA), cannabielsoic acid B (CBEA-B), cannabielsoin (CBE), cannabielsoin acid A (CBEA-A), cannabigerol (CBG), cannabigerol monomethylether (CBGM), cannabigerolic acid (CBGA), cannabigerolic acid monomethylether (CBGAM), cannabigerovarin (CBGV), cannabigerovarinic acid (CBGVA), cannabinodiol (CBND), cannabinodivarin (CBVD), cannabinol (CBN), cannabinol methylether (CBNM), cannabinol-C2 (CBN-C2), cannabinol-C4 (CBN-C4), cannabinolic acid (CBNA), cannabiorcool (CBN-C1), cannabivarin (CBV), 10-Ethoxy-9-hydroxy-delta-6a-tetrahydrocannabinol, 8,9-Dihydroxy-delta-6a-tetrahydrocannabinol, cannabitriol (CBT), cannabitriolvarin (CBTV), delta-8-tetrahydrocannabinol (Δ8-THC), delta-8-tetrahydrocannabinolic acid (Δ8-THCA), delta-9-tetrahydrocannabinol (THC), delta-9-tetrahydrocannabinol-C4 (THC-C4), delta-9-tetrahydrocannabinolic acid A (THCA-A), delta-9-tetrahydrocannabinolic acid B (THCA-B), delta-9-tetrahydrocannabinolic acid-C4 (THCA-C4), delta-9-tetrahydrocannabiorcol (THC-C1), delta-9-tetrahydrocannabiorcolic acid (THCA-C1), delta-9-tetrahydrocannabivarin (THCV), delta-9-tetrahydrocannabivarinic acid (THCVA), 10-Oxo-delta-6a-tetrahydrocannabinol (OTHC), cannabichromanon (CBCF), cannabifuran (CBF), cannabiglendol, cannabiripsol (CBR), cannabicitran (CBT), ehydrocannabifuran (DCBF), delta-9-cis-tetrahydrocannabinol (cis-THC), and tryhydroxy-delta-9-tetrahydrocannabinol (triOH-THC).

Suitable methods for measuring the cannabinoid and terpene content in the hemp extract are known in the art. In an embodiment, cannabinoid content is determined using liquid chromatography with mass spectrometry detection (LC-MS). In another embodiment, terpene content is determined using gas chromatography with flame ionization detection (GC-FID) analysis of headspace.

As used herein, the term “flavoring agent” refers to an ingredient that is added to a composition to impart a particular flavor, smell, or other organoleptic property. The flavoring agent can be natural or artificial.

As used herein, the term “oil” refers to a nonpolar viscous liquid that is both hydrophobic and lipophilic. Oils may be isolated from animal, vegetable, or petrochemical products.

As used herein, the term “chew” refers to a product or a portion thereof that has rheological and other texture and organoleptic properties which tend to promote chewing upon the article by a target subject. Generally speaking, a chewable matrix will exhibit sufficient ductility that it is at least slightly malleable when bitten by the target subject and sufficient palatability that the target subject is not deterred by its taste from biting it multiple times. By contrast, “chewable” does not mean merely that an article can be chewed by a subject (i.e., it does not mean merely that some portion of the article will fit within a subject's mouth sufficiently to permit engagement of the subject's teeth against the portion).

The “maximal serum concentration level” of a substance, as used herein, refers to the maximal level of the substance found in a plasma sample following a single administration.

As used herein, the term “cold extrusion” refers to a process for producing edible food products comprising several unit operations including adding, mixing, kneading, pressing, shearing, shaping, and forming, all of which are conducted at or near ambient temperature. As used herein, the term “psychotropic effect” refers to a modification of brain function that results in an alteration of perception, mood, consciousness, and/or behavior.

Overview

The embodiments disclosed herein provide non-naturally occurring or engineered compositions and methods comprising one or more hemp extracts for the treatment of diseases, disorders, syndromes, and/or conditions in animals in need. The diseases, disorders, syndromes, and/or conditions comprise for example, without limitation, inflammation, periuria, anxiety, depression, insomnia, pain (e.g., chronic pain, non-chronic pain, neuropathic pain, neurological dysfunction pain, nociceptive pain, post-operation pain), skin disorders, cancer, psychotic disorders, seizure, epilepsy, osteoarthritis, lymphoma, atopy, allergies, diarrhea (e.g., idiopathic diarrhea), noise aversion, feather plucking, hair pulling, skin wounds, pyoderma, gastrointestinal conditions, behavioral issues, obsessive behaviors, migraines, headaches, insect bites, diabetes, inflammatory bowel disease, dermatological conditions (e.g., pruritus, pyoderma), urinary conditions, anxiety, or frustration. Clinical trials and pharmacokinetic data regarding administering and dosing in treatments are disclosed herein.

Regarding the treatment of inflammation, the endocannabinoid receptor system is known to play a role in pain modulation and attenuation of inflammation. Cannabinoid receptors (CB1 and CB2) are widely distributed throughout the central and peripheral nervous system and are also present in the synovium. However, the psychotropic effects of certain cannabinoids prevent extensive research into their use as single agents for pain relief. The cannabinoids are a group of as many as 60 different compounds that may or may not act at CB receptors. One class of cannabinoids, cannabidiol (CBD), may actually be an antagonist of the CB receptors. In lower vertebrates, CBD can also have immunomodulatory, anti-hyperalgesic, antinociceptive, anti-anxiety, and anti-inflammatory actions, making it an attractive therapeutic option in animals.

Regarding the treatment of periuria (urinary house-soiling problems), this condition in cats can be divided into those related to latrine behavior and those related to marking. Chronic pain and anxiety/frustration may be indicated in both of these conditions. Marking is commonly seen as a response to a threat to the key resources within cat's core area, and latrine related problems commonly arise from issues relating to access to what the cat perceives as an appropriate latrine (Barcelos et al., (2018) Front. Vet. Sci. 5:108). Both of these can be seen as limitations to the cat's autonomy and therefore likely to induce frustration. This may be combined with anxiety as the animal either perceives a physical threat from other individuals (in the case of marking), or is potentially distressed by the lack of access to a desirable latrine. In addition, anxiety may occur at the prospect of discomfort during elimination, if the cat has some painful condition of the urinary system. A recent review indicated that cats with periuria may be around 4 times more likely to have a history of urinary tract disease. (Barcelos et al., 2018). It has recently reported that cats may change posture from squatting to spraying or vice versa, should house-soiling become uncomfortable (Ramos et al., (2018) J Feline Med Surg. doi: 10.1177/1098612X18801034). It is also worth noting that around 40% of superficially healthy subjects with periuria may have a medical problem detectable upon initial clinical examination, with the figure rising to 66.7% for spraying cats and 56.5% for latrining cats upon closer medical evaluation (Ramos et al., 2018).

Regarding the treatment of migraine, this condition is a common neurovascular disorder manifesting itself in attacks of headaches that can reach a level of severe pain in many patients, leading to substantial functional impairment. The recent Global Burden of Disease Study 2010 (GBD2010), conducted by the World Health Organization, estimates a worldwide prevalence of migraine of 14.7%, ranking it third place among the most common diseases, seventh place among specific causes of disability, and top among neurological disorders as cause of total years lived with disability. Migraine, thus, affects millions of people. To date, the pathophysiology of migraine is not fully understood.

Regarding the treatment of neurological disorders, the use of cannabinoid derivatives in the treatment of a variety of neurological disorders in humans has recently been explored, particularly in the treatment of chronic pain and epilepsy. Full spectrum cannabinoid rich industrial hemp-based nutraceuticals (HBN) below 0.3% THC have been shown to have no psychotropic effects and modest activity through non-cannabinoid receptor routes affecting the serotonergic, glycinergic and GABA neurotransmission pathways that may be able to diminish pain, as well as inflammation. Recent research using full spectrum cannabinoid rich HBNs has revealed efficacy of these products in dogs with chronic pain, e.g., Gamble L-J, Boesch J M, Frye C W, Schwark W S, Mann S, Wolfe L, Brown H, Berthelsen E S and Wakshlag J J (2018), Pharmacokinetics, Safety, and Clinical Efficacy of Cannabidiol Treatment in Osteoarthritic Dogs, Front. Vet. Sci. 5:165, DOI 10.3389/fvets.2018.00165, which is incorporated herein by reference in its entirety. Additionally, investigation in epilepsy in humans and the release of EPIDIOLEX®, available from Greenwich Biosciences, Carlsbad, California, as viable treatment shows merits to cannabidiol in the treatment of epilepsy.

Regarding the treatment of cancer, cannabinoids have been studied and utilized for the palliation of cancer symptoms or for the treatment of side effects related to standard cancer therapies (e.g., Eyal Meiri, Haresh Jhangiani, James J. Vredenburgh, Luigi M. Barbato, Frederick J. Carter, Hwa-Ming Yang, and Vickie Baranowski, Efficacy of dronabinol alone and in combination with ondansetron versus ondansetron alone for delayed chemotherapy-induced nausea and vomiting, 2007, Current Medical Research and Opinion, 23:3, 533-543, DOI: 10.1185/030079907X167525; Gonzalez-Rosales F and Walsh D, Intractable nausea and vomiting due to gastrointestinal mucosal metastases relieved by tetrahydrocannabinol (dronabinol), J Pain Symptom Manage, 1997 November, 14(5), pp. 311-4, doi: 10.1016/S0885-3924(97)00229-7; and May M B and Glode A E, Dronabinol for chemotherapy-induced nausea and vomiting unresponsive to antiemetics, Cancer Manag. Res., 2016 May, 12(8), pp. 49-55, doi: 10.2147/CMAR.S81425, which are incorporated herein by reference in their entirety). However, more recent studies have been exploring CBD and its direct antineoplastic properties alone or in combination with standard cancer therapies, such as chemotherapy or ionizing radiation (e.g., Sánchez C, de Ceballos M L, Gomez del Pulgar T, Rueda D, Corbacho C, Velasco G, Galve-Roperh I, Huffman J W, Ramón y Cajal S, and Guzmán M, Inhibition of glioma growth in vivo by selective activation of the CB(2) cannabinoid receptor, Cancer Res, 2001 Aug. 1, 61(15), 5784-9; Scott K A, Dalgleish A G, and Liu W M, The combination of cannabidiol and Δ9-tetrahydrocannabinol enhances the anticancer effects of radiation in an orthotopic murine glioma model, Mol. Cancer Ther, 2014 December, 13(12), pp. 2955-67, doi: 10.1158/1535-7163; Deng L, Ng L, Ozawa T, and Stella N, Quantitative Analyses of Synergistic Responses between Cannabidiol and DNA Damaging Agents on the Proliferation and Viability of Glioblastoma and Neural Progenitor Cells in Culture, J. Pharmacol. Exp. Ther., 2017 January, 360(1), pp. 215-224, doi: 10.1124/jpet.116.236968; and Luciano De Petrocellis, Alessia Ligresti, Aniello Schiano Moriello, Mariagrazia Iappelli, Roberta Verde, Colin G Stott, Luigia Cristino, Pierangelo Orlando, and Vincenzo Di Marzo, Non-THC cannabinoids counteract prostate carcinoma growth in vitro and in vivo: pro-apoptotic effects and underlying mechanisms. Br. J. Pharmacol. 2012, 168, pp. 79-102, which are incorporated herein by reference in their entirety). Equally is the ability for CBD to activate GPCR18, and GPCR55 whose actions are poorly elucidated in cancer cell biology (e.g., Brown A J and Robin Hiley C, Is GPR55 an anandamide receptor?, Vitam. Horm., 2009, 81, pp. 111-37, doi: 10.1016/S0083-6729(09)81005-4; and De Petrocellis L and Di Marzo V, Non-CB1, non-CB2 receptors for endocannabinoids, plant cannabinoids, and synthetic cannabimimetics: focus on G-protein-coupled receptors and transient receptor potential channels, J. Neuroimmune Pharmacol., 2010 March, 5(1), pp. 103-21, doi: 10.1007/s11481-009-9177-z, which are incorporated herein by reference in their entirety) but appear to control ion channels or incite activation of signalling cascades (e.g., Ross R A, The enigmatic pharmacology of GPR55, Trends Pharmacol. Sci., 2009 March, 30(3), pp. 156-63. doi: 10.1016/j.tips.2008.12.004; and Console-Bram L, Brailoiu E, Brailoiu G C, Sharir H, and Abood M E, Activation of GPR18 by cannabinoid compounds: a tale of biased agonism, Br. J. Pharmacol., 2014 August, 171(16), pp. 3908-17, doi: 10.1111/bph.12746, which are incorporated herein by reference in their entirety).

Still regarding the treatment of cancer, glioblastoma multiforme has been a major focus of cannabinoid-based research in human tumor models. These studies have demonstrated a reduction in the cell viability of glioma cell lines treated with CBD, as well as synergetic reductions in cell viability in combination with ionizing radiation and/or DNA-damaging agents both in vitro and in xenograft murine models (e.g., Scott K A, Dalgleish A G, and Liu W M, The combination of cannabidiol and Δ9-tetrahydrocannabinol enhances the anticancer effects of radiation in an orthotopic murine glioma model, Mol Cancer Ther., 2014, 13(12), pp. 2955-2967; Deng L, Ng L, Ozawa T, and Stella N, Quantitative analysis of synergistic responses between cannabidiol and DNA-damaging agents on the proliferation and viability of glioblastoma and neural progenitor cells in culture, J Pharmacol Exp Ther, 2017, 360(1), pp. 215-224; and Marcu J P, Christian R T, Lau D, Zielinski A J, Horowitz M P, Lee J, Pakdel A, Allison J, Limbad C, Moore D H, Yount G L, Desprez P Y, and McAllister S D, Cannabidiol enhances the inhibitory effects of delta9-tetrahydrocannabinol on human glioblastoma cell proliferation and survival, Mol Cancer Ther, 2010 January, 9(1), pp. 180-9, doi: 10.1158/1535-7163, which are incorporated herein by reference in their entirety). Induction of apoptosis has been observed in many cell culture models and there appear to be numerous cell signalling pathways affected leading to apoptosis and/or autophagy, namely the mammalian target of rapamycin (mTOR), phosphtidyl-inositol-3 kinase (PI3K) and mitogen-activated protein (MAP) kinases (e.g. Sultan A S, Marie M A, and Sheweita S A, Novel mechanism of cannabidiol-induced apoptosis in breast cancer cell lines, Breast, 2018, 41, pp. 34-41; Roperh I, Sanchez C, and Cortes M, Anti-tumoral action of cannabinoids: involvement of sustained ceramide accumulation and extracellular signal-related kinase activation, Nature Med, 2000, 6, pp. 313-9; Powles T, to Poele R, Shamash J, Chaplin T, Propper D, Joel S, Oliver T, and Liu W M, Cannabis-induced cytotoxicity in leukemic cell lines: the role of the cannabinoid receptors and the MAPK pathway, Blood, 2005 Feb. 1, 105(3), pp. 1214-21, doi: 10.1182/blood-2004-03-1182; Ellert-Maklaszemska A, Kaminsha B, and Konarska L. Cannabinoids downregulate PI3K/Akt and Erk signaling pathways and activate proapoptotic function of bad protein, Cell Signal., 2005, 17, pp. 25-37; and Sarker K P, Biswas K K, Yamakuchi M, Lee K Y, Hahiguchi T, Kracht M, Kitajima I, and Maruyama I, ASK1-p38 MAPK/JNK signaling cascade mediates anandamide-induced PC12 cell death, J Neurochem, 2003 April, 85(1), pp. 50-61, doi: 10.1046/j.1471-4159.2003.01663.x, which are incorporated herein by reference in their entirety). To date, there has been little to no examination of the effects of CBD on canine cell culture models or the effects of standard CBD-rich hemp extracts.

Still regarding the treatment of cancer, the use of hemp extracts in controlling cellular growth is complex considering hemp extracts with relatively similar CBD concentrations appear to have differential effects on cell culture systems lending to the proposition of the “entourage effect” whereby terpenes and other cannabinoids may be acting synergistically with CBD to influence cell proliferation (e.g., Russo E B and Taming THC, Potential cannabis synergy and phytocannabinoid-terpenoid entourage effects, Brit J Pharmacol, 2011, 163(7), pp. 1344-1364; Blasco-Benito S, Seijo-Vila M, Caro-Villalobos M, Tundidor I, Andradas C, Garcia-Taboada E, Wade J, Smith S, Guzmán M, Pérez-Gómez E, Gordon M, and Sánchez C, Appraising the “entourage effect”: Antitumor action of a pure cannabinoid versus a botanical drug preparation in preclinical models of breast cancer, Biochem Pharmacol, 2018 November, 157, pp. 285-293, doi: 10.1016/j.bcp.2018.06.025; and Ben-Shabat S, Fride E, Sheskin T, Tamiri T, Rhee M H, Vogel Z, Bisogno T, De Petrocellis L, Di Marzo V, and Mechoulam R, An entourage effect: inactive endogenous fatty acid glycerol esters enhance 2-arachidonoyl-glycerol cannabinoid activity, European journal of pharmacology, 1998 Jul. 17, 353(1), pp. 23-31, which are incorporated herein by reference in their entirety). The cell death response can be in conjunction with chemotherapeutic agents commonly used in veterinary treatment, as many owners want to utilize CBD-rich hemp products during chemotherapy for the effects on the cancer itself, or to relieve some the adverse effects of chemotherapy (nausea and lethargy) to maintain or improve their pet's quality of life (e.g., Kogan L R, Hellyer P W, and Robinson N G, Consumers' perceptions of hemp products for animals, J Am Holist Vet Med Assoc., 2016, 42, pp. 40-48, which is incorporated herein by reference in its entirety).

Regarding the treatment of lymphoma, the disease is one of the most commonly diagnosed hematopoietic cancers in dogs. The most common chemotherapy protocol used for this disease is a doxorubicin-based multidrug protocol (L-asparaginase, cyclophosphamide, doxorubicin, vincristine, and prednisone). Remission rates for such protocols range from 80-90%. Median survival time in dogs diagnosed with lymphoma treated with a doxorubicin based chemotherapy protocol ranges from 6 to 12 months. Approximately 20% to up to 50% of dogs undergoing CHOP or L-CHOP chemotherapy may experience variable degree of GI toxicity (Tomiyasu et al. (2010) J Vet Med Sci. 72(11):1391-7; Mason et al. (2014) J Small Anim Pract. 55(8):391-8; which are incorporated herein by reference in their entirety). The availability of intensive therapeutic modalities and increased client willingness to pursue treatment has led to improved survival times in veterinary cancer patients. However, with more intensive therapy, the risk of associated morbidity has also increased and balancing quality of life with quantity of life has become an important role for the veterinary oncologist. Owners of pets with terminal illness tend to value quality of life (QoL) over longevity and are willing to trade survival time to preserve QoL (Iliopoulou et al. (2013) J Am Vet Med Assoc. 242(12):1679-87; Giuffrida et al. (2018) J Amer Vet Med Assoc. 252:1073-1083, which are incorporated herein by reference in their entirety)

Pharmaceutical Compositions

In an aspect, provided herein is a pharmaceutical composition comprising hemp extract and a carrier, wherein the hemp extract comprises:
cannabidiol; and
cannabidiolic acid.

In another embodiment, the hemp extract comprises:
cannabidiol;
cannabidiolic acid;
cannabigerolic acid;
Δ9-tetrahydrocannabinol; and
cannabichromene.

In another embodiment, the hemp extract comprises:
cannabidiol;
cannabidiolic acid;
cannabigerolic acid;
cannabigerol;
Δ9-tetrahydrocannabinol; and
cannabichromene.

In another embodiment, the ratio of Δ9-tetrahydrocannabinol to the other cannabinoids is from about 1:50 to about 1:20. In an embodiment, the ratio of cannabidiol to cannabidiolic acid is about 0.1:1 to about 1:0.1. In another embodiment, the ratio of cannabidiol to cannabidiolic acid is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1. In yet another embodiment, the ratio of cannabidiol to cannabidiolic acid is about 0.6:1 to about 1:0.6. In still another embodiment, the ratio of cannabidiol to cannabidiolic acid is about 1:1.

In an embodiment, the concentration of Δ9-tetrahydrocannabinol is insufficient to produce a psychotropic effect. In another embodiment, the ratio of Δ9-tetrahydrocannabinol to the other cannabinoids is from about 1:50 to about 1:20. In yet another embodiment, the ratio of Δ9-tetrahydrocannabinol to the other cannabinoids is about 1:50. In still another embodiment, the ratio of Δ9-tetrahydrocannabinol to the other cannabinoids is about 1:45. In an embodiment, the ratio of Δ9-tetrahydrocannabinol to the other cannabinoids is about 1:40. In another embodiment, the ratio of Δ9-tetrahydrocannabinol to the other cannabinoids is about 1:35. In yet another embodiment, the ratio of Δ9-tetrahydrocannabinol to the other cannabinoids is about 1:30. In still another embodiment, the ratio of Δ9-tetrahydrocannabinol to the other cannabinoids is about 1:25. In an embodiment, the ratio of Δ9-tetrahydrocannabinol to the other cannabinoids is about 1:20.

In an embodiment, the concentration of Δ9-tetrahydrocannabinol is less than about 2 mg/mL. In another embodiment, the concentration of Δ9-tetrahydrocannabinol is less than about 1.5 mg/mL. In yet another embodiment, the concentration of Δ9-tetrahydrocannabinol is less than about 1 mg/mL. In still another embodiment, the concentration of Δ9-tetrahydrocannabinol is less than about 0.9 mg/mL. In yet another embodiment, the concentration of Δ9-tetrahydrocannabinol is less than about 0.8 mg/mL. In an embodiment, the concentration of Δ9-tetrahydrocannabinol is less than about 0.7 mg/mL. In another embodiment, the concentration of Δ9-tetrahydrocannabinol is less than about 0.6 mg/mL. In yet another embodiment, the concentration of Δ9-tetrahydrocannabinol is less than about 0.5 mg/mL. In still another embodiment, the concentration of Δ9-tetrahydrocannabinol is less than about 0.4 mg/mL. In an embodiment, the concentration of Δ9-tetrahydrocannabinol is less than about 0.3 mg/mL. In another embodiment, the concentration of Δ9-tetrahydrocannabinol is less than about 0.2 mg/mL. In yet another embodiment, the concentration of Δ9-tetrahydrocannabinol is less than about 0.1 mg/mL. In another embodiment, the concentration of Δ9-tetrahydrocannabinol is less than about 0.05 mg/mL. In another embodiment, the concentration of Δ9-tetrahydrocannabinol is about 0 mg/mL.

In an embodiment, the hemp extract comprises:
about 0.1-20 mg/mL of cannabidiol;
about 0.1-20 mg/mL of cannabidiolic acid;
about 0.01-0.5 mg/mL cannabigerolic acid;
about 0.01-0.5 mg/mL Δ9-tetrahydrocannabinol; and/or
about 0.01-0.5 mg/mL cannabichromene.

In another embodiment, the hemp extract comprises:
about 1-10 mg/mL of cannabidiol;
about 1-10 mg/mL of cannabidiolic acid;
about 0.05-0.2 mg/mL cannabigerolic acid;
about 0.1-0.3 mg/mL Δ9-tetrahydrocannabinol; and/or
about 0.1-0.4 mg/mL cannabichromene.

In yet another embodiment, the hemp extract comprises:
about 5 mg/mL of cannabidiol;
about 5 mg/mL of cannabidiolic acid;
about 0.11 mg/mL cannabigerolic acid;
about 0.25 mg/mL Δ9-tetrahydrocannabinol; and/or
about 0.27 mg/mL cannabichromene.

In an embodiment, the hemp extract comprises:
about 0.1-20 mg/mL of cannabidiol;
about 0.1-20 mg/mL of cannabidiolic acid;
about 0.01-0.5 mg/mL cannabigerolic acid;
cannabigerol;
about 0.01-0.5 mg/mL Δ9-tetrahydrocannabinol; and/or
about 0.01-0.5 mg/mL cannabichromene.

In another embodiment, the hemp extract comprises:
about 1-10 mg/mL of cannabidiol;
about 1-10 mg/mL of cannabidiolic acid;
about 0.05-0.2 mg/mL cannabigerolic acid;
cannabigerol;
about 0.1-0.3 mg/mL Δ9-tetrahydrocannabinol; and/or
about 0.1-0.4 mg/mL cannabichromene.

In yet another embodiment, the hemp extract comprises:
about 5 mg/mL of cannabidiol;
about 5 mg/mL of cannabidiolic acid;
about 0.11 mg/mL cannabigerolic acid;
cannabigerol;
about 0.25 mg/mL Δ9-tetrahydrocannabinol; and/or
about 0.27 mg/mL cannabichromene.

In some embodiments, the hemp extract comprises tetrahydrocannabinolic acid (THCA).

In an embodiment, the hemp extract comprises about 0.01 mg/mL of cannabinoids. In an embodiment, the hemp extract comprises about 0.05 mg/mL of cannabinoids. In an embodiment, the hemp extract comprises about 0.1 mg/mL of cannabinoids. In an embodiment, the hemp extract comprises about 0.5 mg/mL of cannabinoids. In an embodiment, the hemp extract comprises about 1 mg/mL of cannabinoids. In an embodiment, the hemp extract comprises about 2 mg/mL of cannabinoids. In an embodiment, the hemp extract comprises about 3 mg/mL of cannabinoids. In an embodiment, the hemp extract comprises about 4 mg/mL of cannabinoids. In an embodiment, the hemp extract comprises about 5 mg/mL of cannabinoids. In an embodiment, the hemp extract comprises about 10 mg/mL of cannabinoids. In an embodiment, the hemp extract comprises about 20 mg/mL of cannabinoids. In an embodiment, the hemp extract comprises about 30 mg/mL of cannabinoids. In an embodiment, the hemp extract comprises about 40 mg/mL of cannabinoids. In an embodiment, the hemp extract comprises about 50 mg/mL of cannabinoids. In an embodiment, the hemp extract comprises about 60 mg/mL of cannabinoids. In an embodiment, the hemp extract comprises about 70 mg/mL of cannabinoids. In an embodiment, the hemp extract comprises about 80 mg/mL of cannabinoids. In an embodiment, the hemp extract comprises about 90 mg/mL of cannabinoids. In an embodiment, the hemp extract comprises about 100 mg/mL of cannabinoids. In an embodiment, the cannabinoids are cannabidiol and cannabidiolic acid. According to some embodiments, about 0.05-0.5 mL of the extract is administered topically.

In an embodiment, provided herein is a pharmaceutical composition comprising hemp extract and a carrier, wherein the hemp extract comprises:
α-pinene;
β-myrcene;
β-pinene;
δ-limonene;
linalool;
β-caryophyllene;
α-humulene;
nerolidol 2;
guaiol;
caryophyllene oxide; and/or
α-bisabolol.

In another embodiment, the hemp extract comprises:
about 0.09-0.13% α-pinene;
about 0.23-0.44% β-myrcene;
about 0.04-0.09% β-pinene;
about 0.05-0.09% δ-limonene;
about 0.03-0.06% linalool;
about 0.04-0.07% β-caryophyllene;
about 0.02-0.04% α-humulene;
0.04-0.07% nerolidol 2;
about 0.02-0.04% guaiol;
about 0.04-0.08% caryophyllene oxide; and/or
about 0.01-0.04% α-bisabolol.

In another embodiment, the hemp extract comprises:
about 0.07-0.30% α-pinene;
about 0.10-0.60% β-myrcene;
about 0.02-0.20% β-pinene;
about 0.03-0.20% δ-limonene;
about 0.01-0.08% linalool;
about 0.03-0.09% β-caryophyllene;
about 0.01-0.06% α-humulene;
about 0.02-0.09% nerolidol 2; and/or
about 0.01-0.06% guaiol;

In another embodiment, the hemp extract comprises:
about 0.01-0.50% α-pinene;
about 0.01-0.90% β-myrcene;
about 0.01-0.50% β-pinene;
about 0.01-0.50% δ-limonene;
about 0.01-0.50% linalool;
about 0.01-0.50% β-caryophyllene;
about 0.01-0.50% α-humulene;
about 0.01-0.50% nerolidol 2;
about 0.01-0.50% guaiol;
about 0.01-0.50% caryophyllene oxide; and/or
about 0.01-0.50% α-bisabolol.

In another embodiment, the hemp extract further comprises:
camphene;
β-ocimene;
eucalyptol;
isopulegol; and/or
nerolidol 1.

In another embodiment, the hemp extract comprises:
about 0.02% camphene;
about 0.02-0.03% β-ocimene;
about 0.02-0.05% eucalyptol;
about 0.02% isopulegol; and/or
about 0.02-0.04% nerolidol 1.

In another embodiment, the hemp extract comprises:
about 0.01-0.04% camphene;
about 0.01-0.05% β-ocimene;
about 0.01-0.07% eucalyptol;
about 0.01-0.04% isopulegol; and/or
about 0.01-0.05% nerolidol 1.

In another embodiment, the hemp extract comprises:
about 0.01-0.50% camphene;
about 0.01-0.50% β-ocimene;
about 0.01-0.50% eucalyptol;
about 0.01-0.50% isopulegol; and/or
about 0.01-0.50% nerolidol 1.

In an embodiment, the hemp extract does not comprise terpenes.

In an embodiment, the hemp extract comprises 1 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 2 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 3 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 4 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 5 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 6 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 7 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 8 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 9 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 10 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 11 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 12 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 13 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 14 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 15 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises the following: α-pinene, β-myrcene, (3-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the composition is formulated as an oil. In another embodiment, the carrier is selected from the group consisting of hemp seed oil, linseed oil, olive oil, fish oil, salmon oil, coconut oil, catnip oil, sesame oil, MCT oil, and grapeseed oil. In yet another embodiment, the carrier is grapeseed oil. In an embodiment, the carrier is sesame oil.

In an embodiment, the dosage form comprises nepetalactone.

In an embodiment, the dosage form comprises taurine.

In an embodiment, the pharmaceutical composition comprises lecithin. In another embodiment, the lecithin is sunflower lecithin. In another embodiment, the sunflower is about 5%, about 10%, about 15%, about 20%, about 25%, about 30%, about 35%, about 40%, about 45%, or 50%.

In an embodiment, the pharmaceutical composition comprises NF-971P. In an embodiment, the NF-971P is about 0.5%, about 1.0%, about 1.5%, about 2.0%, about 2.5%, or about 3.0% weight/volume ratio.

In an embodiment, the pharmaceutical composition is formulated as a sublingual spray. In still another embodiment, the pharmaceutical composition is formulated as a water or alcohol soluble solution, a gel, or a cream for topical or transdermal application. In an embodiment, the pharmaceutical composition is applied to the back of the neck. In an embodiment, the pharmaceutical composition is applied via transdermal aural application. In another embodiment, the pharmaceutical composition is administered at a dose of 4 mg/kg. In another embodiment, the pharmaceutical composition is administered twice daily for four weeks. In an embodiment, the pharmaceutical composition is formulated as a gel for buccal or mucosal administration. In an embodiment, the pharmaceutical composition is formulated as a paste for buccal or mucosal administration. In an embodiment, the pharmaceutical composition is formulated as a powder. In another embodiment, the pharmaceutical composition is formulated as a solution for subcutaneous injection. In yet another embodiment, the pharmaceutical composition is formulated as a tablet. In still another embodiment, the pharmaceutical composition is formulated as a capsule. In an embodiment, the pharmaceutical composition is formulated as a hard chewable. In an embodiment, the pharmaceutical composition is formulated as a soft chewable.

In an embodiment, the composition is formulated as a chew for oral administration. In another embodiment, the chew is produced using cold extrusion. In another embodiment, the weight of the chew is about 0.5-10 g. In yet another embodiment, the weight of the chew is about 4 g, about 6 g, about 9 g, or about 10 g. In still another embodiment, the weight of the chew is about 0.5 g. In an embodiment, the weight of the chew is about 1 g. In another embodiment, the weight of the chew is about 1.5 g. In yet another embodiment, the weight of the chew is about 2 g. In still another embodiment, the weight of the chew is about 3 g. In an embodiment, the weight of the chew is about 4 g. In another embodiment, the weight of the chew is about 5 g. In yet another embodiment, the weight of the chew is about 6 g. In still another embodiment, the weight of the chew is about 7 g. In an embodiment, the weight of the chew is about 8 g. In another embodiment, the weight of the chew is about 9 g. In yet another embodiment, the weight of the chew is about 10 g.

In an embodiment, the 4 g chew comprises:
about 7 mg of cannabidiol;
about 6 mg of cannabidiolic acid;
about 0.12 mg cannabigerolic acid;
about 0.32 mg Δ9-tetrahydrocannabinol; and
about 0.36 mg cannabichromene.

The pharmaceutical compositions of the present disclosure may be manufactured by processes well known in the art, e.g., by means of conventional mixing, dissolving, granulating, grinding, pulverizing, dragee-making, levigating, emulsifying, encapsulating, entrapping or by lyophilizing processes.

The compositions for use in accordance with the present disclosure thus may be formulated in conventional manner using one or more pharmaceutically acceptable carriers comprising excipients and auxiliaries, which facilitate processing of the active compounds into preparations which can be used pharmaceutically. Proper formulation is dependent upon the route of administration chosen.

Dosage Forms

In an aspect, provided herein is a dosage form comprising:
cannabidiol;
cannabidiolic acid; and/or
one or more pharmaceutically acceptable additives, flavoring agents, surfactants, and adjuvants.

In another embodiment, the dosage form comprises:
cannabidiol;
cannabidiolic acid;
cannabigerolic acid;
Δ9-tetrahydrocannabinol;
cannabichromene; and/or
one or more pharmaceutically acceptable additives, flavoring agents, surfactants, and adjuvants.

In another embodiment, the dosage form comprises:
cannabidiol;
cannabidiolic acid;
cannabigerolic acid;
cannabigerol;
Δ9-tetrahydrocannabinol;
cannabichromene; and/or
one or more pharmaceutically acceptable additives, flavoring agents, surfactants, and adjuvants.

In some embodiments, the source of the cannabinoids in the dosage form is a hemp extract or pharmaceutical composition as disclosed herein.

In an embodiment, the ratio of cannabidiol to cannabidiolic acid is selected from the group consisting of about 1:100, about 1:50, about 1:10, and about 1:1. In an embodiment, the ratio of cannabidiol to cannabidiolic acid is about 0.1:1 to about 1:0.1. In another embodiment, the ratio of cannabidiol to cannabidiolic acid is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1. In yet another embodiment, the ratio of cannabidiol to cannabidiolic acid is about 0.6:1 to about 1:0.6. In still another embodiment, the ratio of cannabidiol to cannabidiolic acid is about 1:1.

In an embodiment, the concentration of Δ9-tetrahydrocannabinol is insufficient to produce a psychotropic effect. In another embodiment, the ratio of Δ9-tetrahydrocannabinol to the other cannabinoids is from about 1:50 to about 1:20. In yet another embodiment, the ratio of Δ9-tetrahydrocannabinol to the other cannabinoids is about 1:50. In still another embodiment, the ratio of Δ9-tetrahydrocannabinol to the other cannabinoids is about 1:45. In an embodiment, the ratio of Δ9-tetrahydrocannabinol to the other cannabinoids is about 1:40. In another embodiment, the ratio of Δ9-tetrahydrocannabinol to the other cannabinoids is about 1:35. In yet another embodiment, the ratio of Δ9-tetrahydrocannabinol to the other cannabinoids is about 1:30. In still another embodiment, the ratio of Δ9-tetrahydrocannabinol to the other cannabinoids is about 1:25. In an embodiment, the ratio of Δ9-tetrahydrocannabinol to the other cannabinoids is about 1:20.

In an embodiment, the concentration of Δ9-tetrahydrocannabinol is less than about 2 mg/mL. In another embodiment, the concentration of Δ9-tetrahydrocannabinol is less than about 1.5 mg/mL. In yet another embodiment, the concentration of Δ9-tetrahydrocannabinol is less than about 1 mg/mL. In still another embodiment, the concentration of Δ9-tetrahydrocannabinol is less than about 0.9 mg/mL. In yet another embodiment, the concentration of Δ9-tetrahydrocannabinol is less than about 0.8 mg/mL. In an embodiment, the concentration of Δ9-tetrahydrocannabinol is less than about 0.7 mg/mL. In another embodiment, the concentration of Δ9-tetrahydrocannabinol is less than about 0.6 mg/mL. In yet another embodiment, the concentration of Δ9-tetrahydrocannabinol is less than about 0.5 mg/mL. In still another embodiment, the concentration of Δ9-tetrahydrocannabinol is less than about 0.4 mg/mL. In an embodiment, the concentration of Δ9-tetrahydrocannabinol is less than about 0.3 mg/mL. In another embodiment, the concentration of Δ9-tetrahydrocannabinol is less than about 0.2 mg/mL. In yet another embodiment, the concentration of Δ9-tetrahydrocannabinol is less than about 0.1 mg/mL. In another embodiment, the concentration of Δ9-tetrahydrocannabinol is less than about 0.05 mg/mL. In another embodiment, the concentration of Δ9-tetrahydrocannabinol is about 0 mg/mL.

In an embodiment, the dosage form comprises:
about 0.1-20 mg/mL of cannabidiol;
about 0.1-20 mg/mL of cannabidiolic acid;
about 0.01-0.5 mg/mL cannabigerolic acid;
about 0.01-0.5 mg/mL Δ9-tetrahydrocannabinol; and/or
about 0.01-0.5 mg/mL cannabichromene.

In another embodiment, the dosage form comprises:
about 1-10 mg/mL of cannabidiol;
about 1-10 mg/mL of cannabidiolic acid;
about 0.05-0.2 mg/mL cannabigerolic acid;
about 0.1-0.3 mg/mL Δ9-tetrahydrocannabinol; and/or
about 0.1-0.4 mg/mL cannabichromene.

In yet another embodiment, the dosage form comprises:
about 5 mg/mL of cannabidiol;
about 5 mg/mL of cannabidiolic acid;
about 0.11 mg/mL cannabigerolic acid;
about 0.25 mg/mL Δ9-tetrahydrocannabinol; and/or
about 0.27 mg/mL cannabichromene.

In some embodiments, the hemp extract comprises THCA.

In some embodiments, the dosage form comprises:
α-pinene;
β-myrcene;
β-pinene;
δ-limonene;
linalool;
β-caryophyllene;
α-humulene;
nerolidol 2;
guaiol;
caryophyllene oxide; and/or
α-bisabolol.

In another embodiment, the dosage form comprises:
about 0.09-0.13% α-pinene;
about 0.23-0.44% β-myrcene;
about 0.04-0.09% β-pinene;
about 0.05-0.09% δ-limonene;
about 0.03-0.06% linalool;
about 0.04-0.07% β-caryophyllene;
about 0.02-0.04% α-humulene;
about 0.04-0.07% nerolidol 2;
about 0.02-0.04% guaiol;
about 0.04-0.08% caryophyllene oxide; and/or
about 0.01-0.04% α-bisabolol.

In another embodiment, the dosage form comprises:
about 0.07-0.30% α-pinene;
about 0.10-0.60% β-myrcene;
about 0.02-0.20% β-pinene;
about 0.03-0.20% δ-limonene;
about 0.01-0.08% linalool;
about 0.03-0.09% β-caryophyllene;
about 0.01-0.06% α-humulene;
about 0.02-0.09% nerolidol 2; and/or
about 0.01-0.06% guaiol.

In another embodiment, the dosage form comprises:
about 0.01-0.50% α-pinene;
about 0.01-0.90% β-myrcene;
about 0.01-0.50% β-pinene;
about 0.01-0.50% δ-limonene;
about 0.01-0.50% linalool;
about 0.01-0.50% β-caryophyllene;
about 0.01-0.50% α-humulene;
about 0.01-0.50% nerolidol 2;
about 0.01-0.50% guaiol;
about 0.01-0.50% caryophyllene oxide; and/or about 0.01-0.50% α-bisabolol.

In another embodiment, the dosage form further comprises:
camphene;
β-ocimene;
eucalyptol;
isopulegol; and/or
nerolidol 1.

In another embodiment, the dosage form comprises:
about 0.02% camphene;
about 0.02-0.03% β-ocimene;
about 0.02-0.05% eucalyptol;
about 0.02% isopulegol; and/or
about 0.02-0.04% nerolidol 1.

In another embodiment, the dosage form comprises:
about 0.01-0.04% camphene;
about 0.01-0.05% β-ocimene;
about 0.01-0.07% eucalyptol;
about 0.01-0.04% isopulegol; and/or
about 0.01-0.05% nerolidol 1.

In another embodiment, the dosage form comprises:
about 0.01-0.50% camphene;
about 0.01-0.50% β-ocimene;
about 0.01-0.50% eucalyptol;
about 0.01-0.50% isopulegol; and/or
about 0.01-0.50% nerolidol 1.

In an embodiment, the hemp extract does not comprise terpenes.

In an embodiment, the hemp extract comprises 1 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 2 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 3 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the hemp extract comprises 4 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 5 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 6 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 7 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 8 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 9 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 10 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 11 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 12 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 13 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 14 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises 15 or more of the following: α-pinene, β-myrcene, β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the dosage form comprises the following: α-pinene, β-myrcene, (β-pinene, δ-limonene, linalool, β-caryophyllene, α-humulene, nerolidol 2, guaiol, caryophyllene oxide, α-bisabolol, camphene, β-ocimene, eucalyptol, isopulegol, and nerolidol 1.

In an embodiment, the flavoring agent is selected from the group consisting of peanut butter, catnip oil, peppermint oil, mango extract, beef, poultry, and seafood. In another embodiment, the flavoring agent is peanut butter.

In an embodiment, the dosage form is formulated as a sublingual spray. In still another embodiment, the dosage form is formulated as a water or alcohol soluble solution, a gel, or a cream for topical or transdermal application. In an embodiment, the dosage form is applied to the back of the neck. In an embodiment, the dosage form is applied via transdermal aural application. In another embodiment, the dosage form is administered at a dose of 4 mg/kg. In another embodiment, the dosage form is administered twice daily for four weeks. In an embodiment, the dosage form is formulated as a gel for buccal or mucosal administration. In some embodiments, the dosage form is formulated as a paste for buccal or mucosal administration. In an embodiment, the dosage form is formulated as a powder. In another embodiment, the dosage form is formulated as a solution for subcutaneous injection. In yet another embodiment, the dosage form is formulated as a tablet. In still another embodiment, the dosage form is formulated as a capsule. In an embodiment, the dosage form is formulated as a soft chewable.

In some embodiments, the invention includes infusing edible products with hemp extract. In another embodiment, the edible product is an extruded food product, baked food product, nut butter, spread, pelleted feed, or processed food. In another embodiment, the edible product is a pet food. In another embodiment the edible product is in a dry, shelf-stable form such as dried fish, dried dairy products, fish meal, fish flour, cereals, flours, carbohydrates, dried fruits, etc. In another embodiment, the edible product is moist or semi-moist. In another embodiment, the edible product contains additives or supplements such as vitamins, minerals, medicinals, etc., for example chemicals, enzymes, etc., capable of removing plaque or tartar from the animal's teeth, etc. In an embodiment, the hemp extract is administered with catnip oil. In another embodiment, any of the dosage forms described can also include catnip.

In another embodiment, hemp extracts are administered using a nebulizer. In another embodiment, the nebulizer delivery device and system is capable of effectively and efficiently administering one or more nebulized drug to an animal. In another embodiment, the nebulizer system can easily be used on animals without removing them from their natural environment. In another embodiment, the nebulizer delivery device and system enables animals to be easily treated daily or multiple times a day without undue stress or the need for extensive resources. In another embodiment, the nebulizer delivery device and system can be used on animals having varying levels of training.

In one embodiment, hemp extract is administered using a diffuser. The diffuser can be any device which disperses hemp extract into the air. Hemp extract may be dispersed by any method, including by natural convection, by forced convection, by heating a wick or pad, for example, holding the hemp extract, by using pumps, or with fans.

In one embodiment, hemp extract is administered by a pet collar. The pet collar may comprise a belt with a buckle on one side, a free end on the other side and an attachment means, such as apertures disposed longitudinally within the central portion of the belt, or a quick release clasp mechanism, for securing the collar in a closed loop configuration. The pet collar may be made from a variety of materials including nylon, polyester leather or other suitable material. The belt material may be treated with a water-proofing compound. The nylon or polyester belt may be interwoven with reflective fibers to enhance the visibility of the pet collar during nighttime hours. In one embodiment, the collar is infused with hemp extract.

Chews

In an embodiment, the dosage form is formulated as a chew for oral administration. In another embodiment, the chew is produced using cold extrusion. In another embodiment, the weight of the chew is about 0.5-10 g. In yet another embodiment, the weight of the chew is about 4 g, about 6 g, about 9 g, or about 10 g. In still another embodiment, the weight of the chew is about 0.5 g. In an embodiment, the weight of the chew is about 1 g. In another embodiment, the weight of the chew is about 1.5 g. In yet another embodiment, the weight of the chew is about 2 g. In still another embodiment, the weight of the chew is about 3 g. In an embodiment, the weight of the chew is about 4 g. In another embodiment, the weight of the chew is about 5 g. In yet another embodiment, the weight of the chew is about 6 g. In still another embodiment, the weight of the chew is about 7 g. In an embodiment, the weight of the chew is about 8 g. In another embodiment, the weight of the chew is about 9 g. In yet another embodiment, the weight of the chew is about 10 g.

In one embodiment, the dosage form comprises:
brewer's yeast;
arabic gum;
guar gum;
a flavoring agent;
Verdilox;
Previon;
hemp extract;
glycerin;
sunflower lecithin; and/or
water.

In another embodiment, the dosage form comprises:
about 25-35% brewer's yeast;
about 1-10% arabic gum;
about 0.1-4% guar gum;
about 10-20% of a flavoring agent;
about 0.01-1% Verdilox;
about 0.1-2% Previon;
about 1-10% hemp extract;
about 10-20% glycerin;
about 1-10% sunflower lecithin; and/or
about 1-10% water.

In another embodiment, the dosage form comprises:
about 29-33% brewer's yeast;
about 3-6% arabic gum;
about 0.5-2% guar gum;
about 12-16% of a flavoring agent;
about 0.01-0.1% Verdilox;
about 0.5-1.5% Previon;
about 3-6% hemp extract;
about 13-17% glycerin;
about 3-7% sunflower lecithin; and/or
about 3-7% water.

In yet another embodiment, the dosage form comprises:
about 30% brewer's yeast;
about 4.7% arabic gum;
about 0.9% guar gum;
about 14.2% of a flavoring agent;
about 0.05% Verdilox;
about 0.9% Previon;
about 4.7% hemp extract;
about 15.1% glycerin;
about 5.7% sunflower lecithin; and/or
about 5.7% water.

In one embodiment, the dosage form comprises:
glucosamine HCl;
brewer's yeast;
arabic gum;
guar gum;
a flavoring agent;
Verdilox;
Previon;
hemp extract;
glycerin;
sunflower lecithin; and/or
water.

In another embodiment, the dosage form comprises:
about 10-20% glucosamine HCl;
about 25-35% brewer's yeast;
about 1-10% arabic gum;
about 0.1-4% guar gum;
about 10-20% of a flavoring agent;
about 0.01-1% Verdilox;
about 0.1-2% Previon;
about 1-10% hemp extract;
about 10-20% glycerin;
about 1-10% sunflower lecithin; and/or
about 1-10% water.

In another embodiment, the dosage form comprises:
about 12-17% glucosamine HCl;
about 29-33% brewer's yeast;
about 3-6% arabic gum;
about 0.5-2% guar gum;
about 12-16% of a flavoring agent;
about 0.01-0.1% Verdilox;
about 0.5-1.5% Previon;
about 3-6% hemp extract;
about 13-17% glycerin;
about 3-7% sunflower lecithin; and/or
about 3-7% water.

In yet another embodiment, the dosage form comprises:
about 15.6% glucosamine HCl;
about 30% brewer's yeast;
about 4.7% arabic gum;
about 0.9% guar gum;
about 14.2% of a flavoring agent;
about 0.05% Verdilox;
about 0.9% Previon;
about 4.7% hemp extract;
about 15.1% glycerin;
about 5.7% sunflower lecithin; and/or
about 5.7% water.

In one embodiment, the dosage form comprises:
glucosamine HCl;
chondroitin sulfate (76%);
brewer's yeast;
arabic gum;
guar gum;
a flavoring agent;
Verdilox;
Previon;
hemp extract;
glycerin;
sunflower lecithin; and/or
water.

In another embodiment, the dosage form comprises:
about 10-20% glucosamine HCl;
about 0.1-7% chondroitin sulfate (76%);
about 25-35% brewer's yeast;
about 1-10% arabic gum;
about 0.1-4% guar gum;
about 10-20% of a flavoring agent;
about 0.01-1% Verdilox;
about 0.1-2% Previon;
about 1-10% hemp extract;
about 10-20% glycerin;
about 1-10% sunflower lecithin; and/or
about 1-10% water.

In another embodiment, the dosage form comprises:
about 12-17% glucosamine HCl;
about 1-4% chondroitin sulfate (76%);
about 29-33% brewer's yeast;
about 3-6% arabic gum;
about 0.5-2% guar gum;
about 12-16% of a flavoring agent;
about 0.01-0.1% Verdilox;
about 0.5-1.5% Previon;
about 3-6% hemp extract;
about 13-17% glycerin;
about 3-7% sunflower lecithin; and/or
about 3-7% water.

In yet another embodiment, the dosage form comprises:
about 15.6% glucosamine HCl;
about 2.6% chondroitin sulfate (76%);
about 30% brewer's yeast;
about 4.7% arabic gum;
about 0.9% guar gum;
about 14.2% of a flavoring agent;
about 0.05% Verdilox;
about 0.9% Previon;
about 4.7% hemp extract;
about 15.1% glycerin;
about 5.7% sunflower lecithin; and/or
about 5.7% water.

In another embodiment, the dosage form comprises:
hyaluronic acid;
brewer's yeast;
arabic gum;
guar gum;
a flavoring agent;
Verdilox;
Previon;
hemp extract;
glycerin;
sunflower lecithin; and/or
water.

In another embodiment, the dosage form comprises:
about 0.01-3% hyaluronic acid;
about 25-35% brewer's yeast;
about 1-10% arabic gum;
about 0.1-5% guar gum;
about 10-20% of a flavoring agent;
about 0.01-1% Verdilox;
about 0.1-3% Previon;
about 1-10% hemp extract;
about 10-20% glycerin;
about 1-10% sunflower lecithin; and/or
about 1-10% water.

In another embodiment, the dosage form comprises:
about 0.01-1% hyaluronic acid;
about 29-33% brewer's yeast;
about 3-6% arabic gum;
about 0.5-2% guar gum;
about 12-16% of a flavoring agent;
about 0.01-0.1% Verdilox;
about 0.5-1.5% Previon;
about 3-6% hemp extract;
about 13-17% glycerin;
about 3-7% sunflower lecithin; and/or
about 3-7% water.

In yet another embodiment, the dosage form comprises:
about 0.1% hyaluronic acid;
about 30.6% brewer's yeast;
about 4.8% arabic gum;
about 0.97% guar gum;
about 14.5% of a flavoring agent;
about 0.05% Verdilox;
about 0.97% Previon;
about 4.8% hemp extract;
about 15.5% glycerin;
about 5.8% sunflower lecithin; and/or
about 5.8% water.

In another embodiment, the dosage form comprises:
glucosamine HCl;
hyaluronic acid;
brewer's yeast;
arabic gum;
guar gum;
a flavoring agent;
Verdilox;
Previon;
hemp extract;
glycerin;
sunflower lecithin; and/or
water.

In another embodiment, the dosage form comprises:
about 10-20% glucosamine HCl;
about 0.01-3% hyaluronic acid;
about 25-35% brewer's yeast;
about 1-10% arabic gum;
about 0.1-5% guar gum;
about 10-20% of a flavoring agent;
about 0.01-1% Verdilox;
about 0.1-3% Previon;
about 1-10% hemp extract;
about 10-20% glycerin;
about 1-10% sunflower lecithin; and/or
about 1-10% water.

In another embodiment, the dosage form comprises:
about 12-17% glucosamine HCl;
about 0.01-1% hyaluronic acid;
about 29-33% brewer's yeast;
about 3-6% arabic gum;
about 0.5-2% guar gum;
about 12-16% of a flavoring agent;
about 0.01-0.1% Verdilox;
about 0.5-1.5% Previon;
about 3-6% hemp extract;
about 13-17% glycerin;
about 3-7% sunflower lecithin; and/or
about 3-7% water.

In yet another embodiment, the dosage form comprises:
about 16% glucosamine HCl;
about 0.1% hyaluronic acid;
about 30.6% brewer's yeast;
about 4.8% arabic gum;
about 0.97% guar gum;
about 14.5% of a flavoring agent;
about 0.05% Verdilox;
about 0.97% Previon;
about 4.8% hemp extract;
about 15.5% glycerin;
about 5.8% sunflower lecithin; and/or
about 5.8% water.

In yet another embodiment, the dosage form comprises:
hemp extract;
peanut butter;
rice bran;
sweet potato;
dry molasses;
sorbic acid;
brewer's yeast;
sugar;
water;
glycerin;
potato starch;
dehydrated peanut butter;
rice starch; and/or
guar gum.

In another embodiment, the dosage form comprises:
about 3.0-10.0% hemp extract;
about 10.0-20.0% peanut butter;
about 10.0-15.0% rice bran;
about 4.0-10.0% sweet potato;
about 6.0-13.0% dry molasses;
about 0.5-5.0% sorbic acid;
about 2.0-8.0% brewer's yeast;
about 3.0-8.0% sugar;
about 5.0-15.0% water;
about 8.0-18.0% glycerin;
about 1.0-8.0% potato starch;
about 0.5-5.0% dehydrated peanut butter;
about 1.0-5.0% rice starch; and/or
about 1.0-5.0% guar gum.

In another embodiment, the dosage form comprises:
about 5.0% hemp extract;
about 15.0% peanut butter;
about 12.5% rice bran;
about 5.5% sweet potato;
about 8.0% dry molasses;
about 1% sorbic acid;
about 5.0% brewer's yeast;
about 6.0% sugar;
about 9.25% water;
about 13.0% glycerin;
about 2.0% potato starch;
about 1.0% dehydrated peanut butter;
about 2.0% rice starch; and/or
about 2.0% guar gum.

In yet another embodiment, the dosage form comprises:
about 5.0% hemp extract;
about 15.0% peanut butter;
about 13.0% rice bran;
about 6.0% sweet potato;
about 9.0% dry molasses;
about 1% sorbic acid;
about 5.0% brewer's yeast;
about 6.0% sugar;
about 9.5% water;
about 13.0% glycerin;
about 4.0% potato starch;
about 1.0% dehydrated peanut butter;
about 2.0% rice starch; and/or
about 2.0% guar gum.

In an embodiment, the dosage form comprises:
hemp extract;
peanut butter;
rice bran;
glucosamine HCl;
sweet potato;
dry molasses;
sorbic acid;
brewer's yeast;
sugar;
water;
glycerin;
potato starch;
dehydrated peanut butter;
rice starch; and/or
guar gum.

In another embodiment, the dosage form comprises:
about 5.0% hemp extract;
about 15.0% peanut butter;
about 12.5% rice bran;
about 12.75% glucosamine HCl;
about 5.5% sweet potato;
about 8.0% dry molasses;
about 1% sorbic acid;
about 5.0% brewer's yeast;
about 6.0% sugar;
about 9.25% water;
about 13.0% glycerin;
about 2.0% potato starch;
about 1.0% dehydrated peanut butter;
about 2.0% rice starch; and/or
about 2.0% guar gum.

In yet another embodiment, the dosage form comprises:
about 5.0% hemp extract;
about 15.0% peanut butter;
about 13.0% rice bran;
about 8.5% glucosamine HCl;
about 6.0% sweet potato;
about 9.0% dry molasses;
about 1% sorbic acid;
about 5.0% brewer's yeast;
about 6.0% sugar;
about 9.5% water;
about 13.0% glycerin;
about 4.0% potato starch;
about 1.0% dehydrated peanut butter;
about 2.0% rice starch; and/or
about 2.0% guar gum.

In yet another embodiment, the dosage form comprises:
about 3.0-10.0% hemp extract;
about 10.0-20.0% peanut butter;
about 10.0-15.0% rice bran;
about 5.0-15.0% glucosamine HCl;
about 4.0-10.0% sweet potato;
about 6.0-13.0% dry molasses;
about 0.5-5.0% sorbic acid;
about 2.0-8.0% brewer's yeast;
about 3.0-8.0% sugar;
about 5.0-15.0% water;
about 8.0-18.0% glycerin;
about 1.0-8.0% potato starch;
about 0.5-5.0% dehydrated peanut butter;
about 1.0-5.0% rice starch; and/or
about 1.0-5.0% guar gum.

In another embodiment, the dosage form further comprises chondroitin sulfate.

In an embodiment, the dosage form comprises:
hemp extract;
peanut butter;
rice bran;
glucosamine HCl;
sweet potato;
dry molasses;
sorbic acid;
brewer's yeast;
sugar;
water;
glycerin;
potato starch;
dehydrated peanut butter;
DigestaWell PET;
rice starch; and/or
guar gum.

In another embodiment, the dosage form comprises:
about 3.0-10.0% hemp extract;
about 5.0-20.0% peanut butter;
about 10.0-15.0% rice bran;
about 5.0-15.0% glucosamine HCl;
about 4.0-10.0% sweet potato;
about 6.0-13.0% dry molasses;
about 0.5-5.0% sorbic acid;
about 2.0-8.0% brewer's yeast;
about 3.0-8.0% sugar;
about 5.0-15.0% water;
about 8.0-18.0% glycerin;
about 1.0-8.0% potato starch;
about 0.5-5.0% dehydrated peanut butter;
about 0.1-3.0% DigestaWell PET;
about 1.0-8.0% rice starch; and/or
about 1.0-5.0% guar gum.

In another embodiment, the dosage form comprises:
about 5.0% hemp extract;
about 10.0% peanut butter;
about 12.0% rice bran;
about 12.75% glucosamine HCl;
about 5.5% sweet potato;
about 8.0% dry molasses;
about 1% sorbic acid;
about 5.0% brewer's yeast;
about 6.0% sugar;
about 7.25% water;
about 10.0% glycerin;
about 5.0% potato starch;
about 4.0% dehydrated peanut butter;
about 0.5% DigestaWell PET;
about 6.0% rice starch; and/or
about 2.0% guar gum.

In yet another embodiment, the dosage form comprises:
about 5.0% hemp extract;
about 10.0% peanut butter;
about 12.5% rice bran;
about 8.5% glucosamine HCl;
about 8.0% sweet potato;
about 9.0% dry molasses;
about 1% sorbic acid;
about 5.0% brewer's yeast;
about 6.0% sugar;
about 6.0% water;
about 10.0% glycerin;
about 6.0% potato starch;
about 4.0% dehydrated peanut butter;
about 0.5% DigestaWell PET;
about 6.5% rice starch; and/or
about 2.0% guar gum.

In an embodiment, the dosage form comprises:
hemp extract;
peanut butter;
rice bran;
glucosamine HCl;
sweet potato;
dry molasses;
sorbic acid;
brewer's yeast;
sugar;
water;
glycerin;
potato starch;
dehydrated peanut butter;
chondroitin;
DigestaWell PET;
rice starch; and/or
guar gum.

In another embodiment, the dosage form comprises:
about 3.0-10.0% hemp extract;
about 5.0-20.0% peanut butter;
about 10.0-15.0% rice bran;
about 5.0-15.0% glucosamine HCl;
about 4.0-10.0% sweet potato;
about 6.0-13.0% dry molasses;
about 0.5-5.0% sorbic acid;
about 2.0-8.0% brewer's yeast;
about 3.0-8.0% sugar;
about 5.0-15.0% water;
about 8.0-18.0% glycerin;
about 1.0-8.0% potato starch;
about 0.5-5.0% dehydrated peanut butter;
about 0.5-5.0% chondroitin;
about 0.1-3.0% DigestaWell PET;
about 1.0-8.0% rice starch; and/or
about 1.0-5.0% guar gum.

In another embodiment, the dosage form comprises:
about 5.0% hemp extract;
about 10.0% peanut butter;
about 12.0% rice bran;
about 12.75% glucosamine HCl;
about 5.5% sweet potato;
about 8.0% dry molasses;
about 1% sorbic acid;
about 5.0% brewer's yeast;
about 6.0% sugar;
about 7.25% water;
about 10.0% glycerin;
about 4.0% potato starch;
about 4.0% dehydrated peanut butter;
about 2.5% chondroitin;
about 0.5% DigestaWell PET;
about 4.5% rice starch; and/or
about 2.0% guar gum.

In yet another embodiment, the dosage form comprises:
about 5.0% hemp extract;
about 10.0% peanut butter;
about 12.5% rice bran;
about 8.5% glucosamine HCl;
about 8.0% sweet potato;
about 9.0% dry molasses;
about 1% sorbic acid;
about 5.0% brewer's yeast;
about 6.0% sugar;
about 6.0% water;
about 10.0% glycerin;
about 5.0% potato starch;
about 4.0% dehydrated peanut butter;
about 2.5% chondroitin;
about 0.5% DigestaWell PET;
about 5.0% rice starch; and/or
about 2.0% guar gum.

In an embodiment, the dosage form further comprises brewers dried yeast, fructo-oligosaccharides, fumaric acid, lactic acid, citric acid, malic acid, thyme oil, anethole, cinnamaldehyde, vegetable oil, dehydrated alfalfa meal, mineral oil, and/or sodium aluminosilicate.

In another embodiment, the dosage form comprises 2.0% hemp extract. In another embodiment, the dosage form comprises 3.0% hemp extract. In another embodiment, the dosage form comprises 4.0% hemp extract. In another embodiment, the dosage form comprises 5.0% hemp extract. In another embodiment, the dosage form comprises 6.0% hemp extract. In another embodiment, the dosage form comprises 7.0% hemp extract. In another embodiment, the dosage form comprises 8.0% hemp extract. In another embodiment, the dosage form comprises 9.0% hemp extract. In another embodiment, the dosage form comprises 10.0% hemp extract.

In an embodiment, the hemp extract comprises:
cannabidiol;
cannabidiolic acid;
cannabigerolic acid;
Δ9-tetrahydrocannabinol; and/or
cannabichromene.

In an embodiment, the ratio of cannabidiol to cannabidiolic acid is selected from the group consisting of about 1:100, about 1:50, about 1:10, and about 1:1. In an embodiment, the ratio of cannabidiol to cannabidiolic acid is about 0.1:1 to about 1:0.1. In another embodiment, the ratio of cannabidiol to cannabidiolic acid is about 0.1:1, about 0.2:1, about 0.3:1, about 0.4:1, about 0.5:1, about 0.6:1, about 0.7:1, about 0.8:1, about 0.9:1, about 1:1, about 1:0.9, about 1:0.8, about 1:0.7, about 1:0.6, about 1:0.5, about 1:0.4, about 1:0.3, about 1:0.2, or about 1:0.1. In yet another embodiment, the ratio of cannabidiol to cannabidiolic acid is about 0.6:1 to about 1:0.6. In still another embodiment, the ratio of cannabidiol to cannabidiolic acid is about 1:1.

In an embodiment, the concentration of Δ9-tetrahydrocannabinol is insufficient to produce a psychotropic effect. In another embodiment, the ratio of Δ9-tetrahydrocannabinol to the other cannabinoids is from about 1:50 to about 1:20. In yet another embodiment, the ratio of Δ9-tetrahydrocannabinol to the other cannabinoids is about 1:50. In still another embodiment, the ratio of Δ9-tetrahydrocannabinol to the other cannabinoids is about 1:45. In an embodiment, the ratio of Δ9-tetrahydrocannabinol to the other cannabinoids is about 1:40. In another embodiment, the ratio of Δ9-tetrahydrocannabinol to the other cannabinoids is about 1:35. In yet another embodiment, the ratio of Δ9-tetrahydrocannabinol to the other cannabinoids is about 1:30. In still another embodiment, the ratio of Δ9-tetrahydrocannabinol to the other cannabinoids is about 1:25. In an embodiment, the ratio of Δ9-tetrahydrocannabinol to the other cannabinoids is about 1:20.

In an embodiment, the concentration of Δ9-tetrahydrocannabinol is less than about 2 mg/mL. In another embodiment, the concentration of Δ9-tetrahydrocannabinol is less than about 1.5 mg/mL. In yet another embodiment, the concentration of Δ9-tetrahydrocannabinol is less than about 1 mg/mL. In still another embodiment, the concentration of Δ9-tetrahydrocannabinol is less than about 0.9 mg/mL. In yet another embodiment, the concentration of Δ9-tetrahydrocannabinol is less than about 0.8 mg/mL. In an embodiment, the concentration of Δ9-tetrahydrocannabinol is less than about 0.7 mg/mL. In another embodiment, the concentration of Δ9-tetrahydrocannabinol is less than about 0.6 mg/mL. In yet another embodiment, the concentration of Δ9-tetrahydrocannabinol is less than about 0.5 mg/mL. In still another embodiment, the concentration of Δ9-tetrahydrocannabinol is less than about 0.4 mg/mL. In an embodiment, the concentration of Δ9-tetrahydrocannabinol is less than about 0.3 mg/mL. In another embodiment, the concentration of Δ9-tetrahydrocannabinol is less than about 0.2 mg/mL. In yet another embodiment, the concentration of Δ9-tetrahydrocannabinol is less than about 0.1 mg/mL. In another embodiment, the concentration of Δ9-tetrahydrocannabinol is less than about 0.05 mg/mL. In yet another embodiment, the concentration of Δ9-tetrahydrocannabinol is about 0 mg/mL.

In an embodiment, the hemp extract comprises:
about 0.1-20 mg/mL of cannabidiol;
about 0.1-20 mg/mL of cannabidiolic acid;
about 0.01-0.5 mg/mL cannabigerolic acid;
about 0.01-0.5 mg/mL Δ9-tetrahydrocannabinol; and/or
about 0.01-0.5 mg/mL cannabichromene.

In another embodiment, the hemp extract comprises:
about 1-10 mg/mL of cannabidiol;
about 1-10 mg/mL of cannabidiolic acid;
about 0.05-0.2 mg/mL cannabigerolic acid;
about 0.1-0.3 mg/mL Δ9-tetrahydrocannabinol; and/or
about 0.1-0.4 mg/mL cannabichromene.

In yet another embodiment, the hemp extract comprises:
about 5 mg/mL of cannabidiol;
about 5 mg/mL of cannabidiolic acid;
about 0.11 mg/mL cannabigerolic acid;
about 0.25 mg/mL Δ9-tetrahydrocannabinol; and/or
about 0.27 mg/mL cannabichromene.

In some embodiments, the hemp extract comprises THCA.

In an embodiment, the hemp extract comprises:
α-pinene;
β-myrcene;
β-pinene;
δ-limonene;
linalool;
β-caryophyllene;
α-humulene;
nerolidol 2;
guaiol;
caryophyllene oxide; and/or
α-bisabolol.

In another embodiment, the hemp extract comprises:
about 0.09-0.13% α-pinene;
about 0.23-0.44% β-myrcene;
about 0.04-0.09% β-pinene;
about 0.05-0.09% δ-limonene;
about 0.03-0.06% linalool;
about 0.04-0.07% β-caryophyllene;
about 0.02-0.04% α-humulene;
about 0.04-0.07% nerolidol 2;
about 0.02-0.04% guaiol;
about 0.04-0.08% caryophyllene oxide; and/or
about 0.01-0.04% α-bisabolol.

In another embodiment, the hemp extract comprises:
about 0.07-0.30% α-pinene;
about 0.10-0.60% β-myrcene;
about 0.02-0.20% β-pinene;
about 0.03-0.20% δ-limonene;
about 0.01-0.08% linalool;
about 0.03-0.09% β-caryophyllene;
about 0.01-0.06% α-humulene;
about 0.02-0.09% nerolidol 2; and/or
about 0.01-0.06% guaiol;

In another embodiment, the hemp extract comprises:
about 0.01-0.50% α-pinene;
about 0.01-0.90% β-myrcene;
about 0.01-0.50% β-pinene;
about 0.01-0.50% δ-limonene;
about 0.01-0.50% linalool;
about 0.01-0.50% β-caryophyllene;
about 0.01-0.50% α-humulene;
about 0.01-0.50% nerolidol 2;
about 0.01-0.50% guaiol;
about 0.01-0.50% caryophyllene oxide; and/or
about 0.01-0.50% α-bisabolol.

In another embodiment, the hemp extract further comprises:
camphene;
β-ocimene;
eucalyptol;
isopulegol; and/or
nerolidol 1.

In another embodiment, the hemp extract comprises:
about 0.02% camphene;
about 0.02-0.03% β-ocimene;
about 0.02-0.05% eucalyptol;
about 0.02% isopulegol; and/or
about 0.02-0.04% nerolidol 1.

In another embodiment, the hemp extract comprises:
about 0.01-0.04% camphene;
about 0.01-0.05% β-ocimene;
about 0.01-0.07% eucalyptol;
about 0.01-0.04% isopulegol; and/or
about 0.01-0.05% nerolidol 1.

In another embodiment, the hemp extract comprises:
about 0.01-0.50% camphene;
about 0.01-0.50% β-ocimene;
about 0.01-0.50% eucalyptol;
about 0.01-0.50% isopulegol; and/or
about 0.01-0.50% nerolidol 1.

In an embodiment, the composition is formulated as an oil. In another embodiment, the carrier is selected from the group consisting of hemp seed oil, linseed oil, olive oil, fish oil, salmon oil, coconut oil, catnip oil, sesame oil, MCT oil, and grapeseed oil. In yet another embodiment, the carrier is grapeseed oil. In an embodiment, the carrier is sesame oil.

In an embodiment, the flavoring agent is selected from the group consisting of peanut butter, catnip oil, chicken liver powder, poultry extract, maltodextrin, butter, and bacon. In another embodiment, the flavoring agent is chicken liver powder. In another embodiment, the flavoring agent is peanut butter.

In an embodiment, the composition is formulated as a chew for oral administration. In another embodiment, the chew is produced using cold extrusion. In another embodiment, the weight of the chew is about 0.5-10 g. In yet another embodiment, the weight of the chew is about 4 g, about 6 g, about 9 g, or about 10 g. In still another embodiment, the weight of the chew is about 0.5 g. In an embodiment, the weight of the chew is about 1 g. In another embodiment, the weight of the chew is about 1.5 g. In yet another embodiment, the weight of the chew is about 2 g. In still another embodiment, the weight of the chew is about 3 g. In an embodiment, the weight of the chew is about 4 g. In another embodiment, the weight of the chew is about 5 g. In yet another embodiment, the weight of the chew is about 6 g. In still another embodiment, the weight of the chew is about 7 g. In an embodiment, the weight of the chew is about 8 g. In another embodiment, the weight of the chew is about 9 g. In yet another embodiment, the weight of the chew is about 10 g.

In an embodiment, the 4 g chew comprises:
about 7 mg of cannabidiol;
about 6 mg of cannabidiolic acid;
about 0.12 mg cannabigerolic acid;
about 0.32 mg Δ9-tetrahydrocannabinol; and/or
about 0.36 mg cannabichromene.

Methods of Treatment

In an aspect, provided herein is a method for treating a disease, disorder, syndrome, and/or condition in a veterinary subject in need thereof, comprising administering to the veterinary subject a therapeutically effective amount of any of the compositions or dosage forms described above. In an embodiment, the veterinary subject suffers from inflammation, periuria, anxiety, depression, insomnia, pain (e.g., chronic pain, non-chronic pain, neuropathic pain, neurological disfunction pain, nociceptive pain, post-operation pain), a skin disorder, cancer, a psychotic disorder, seizure, epilepsy, osteoarthritis, lymphoma, atopy, an allergy, diarrhea (e.g., idiopathic diarrhea), noise aversion, feather plucking, hair pulling, a skin wound, pyoderma, a gastrointestinal condition, a behavioral issue, obsessive behaviors, a migraine, a headache, insect bites, diabetes, inflammatory bowel disease, dermatological conditions (e.g., pruritus, pyoderma), urinary conditions, anxiety, or frustration.

In an embodiment, the veterinary subject is feline. In an embodiment, the feline is >6 months and <12 years old. In an embodiment, the feline is <6 months old. In an embodiment, the feline is about 6-12 months old. In an embodiment, the feline is about 1-3 years old. In an embodiment, the feline is about 3-6 years old. In an embodiment, the feline is about 6-9 years old. In an embodiment, the feline is about 9-12 years old. In an embodiment, the feline is about 12-15 years old. In an embodiment, the feline is about >15 years old.

In an embodiment, the pharmaceutical composition, dosage form, or hemp extract is administered at a dosage of about 0.1-50.0 mg/kg. In an embodiment, the pharmaceutical composition, dosage form, or hemp extract is administered at a dosage of about 1-50.0 mg/kg. In an embodiment, the pharmaceutical composition, dosage form, or hemp extract is administered at a dosage of about 10-50.0 mg/kg. In an embodiment, the pharmaceutical composition, dosage form, or hemp extract is administered at a dosage of about 10-25 mg/kg. In an embodiment, the pharmaceutical composition, dosage form, or hemp extract is administered at a dosage of about 15-50.0 mg/kg. In an embodiment, the pharmaceutical composition, dosage form, or hemp extract is administered at a dosage of about 15-25 mg/kg. In an embodiment, the pharmaceutical composition, dosage form, or hemp extract is administered at a dosage of about 20-50.0 mg/kg. In an embodiment, the pharmaceutical composition, dosage form, or hemp extract is administered at a dosage of about 25-50.0 mg/kg. In an embodiment, the pharmaceutical composition, dosage form, or hemp extract is administered at a dosage of about 30-50.0 mg/kg. In an embodiment, the pharmaceutical composition, dosage form, or hemp extract is administered at a dosage of about 20-35 mg/kg. In an embodiment, the pharmaceutical composition, dosage form, or hemp extract is administered at a dosage of about 25-35 mg/kg. In an embodiment, the pharmaceutical composition, dosage form, or hemp extract is administered at a dosage of about 35-50.0 mg/kg. In an embodiment, the pharmaceutical composition, dosage form, or hemp extract is administered at a dosage of about 0.1-15.0 mg/kg. In another embodiment, the pharmaceutical composition, dosage form, or hemp extract is administered at a dosage of about 0.1-10.0 mg/kg. In an embodiment, the dosage is given orally. In an embodiment, the dosage is given topically.

In yet another embodiment, the pharmaceutical composition, dosage form, or hemp extract is administered at a dosage of about 0.1 mg/kg. In still another embodiment, the pharmaceutical composition, dosage form, or hemp extract is administered at a dosage of about 0.2 mg/kg. In yet another embodiment, the pharmaceutical composition, dosage form, or hemp extract is administered at a dosage of about 0.3 mg/kg. In an embodiment, the pharmaceutical composition, dosage form, or hemp extract is administered at a dosage of about 0.4 mg/kg. In another embodiment, the pharmaceutical composition, dosage form, or hemp extract is administered at a dosage of about 0.5 mg/kg. In yet another embodiment, the pharmaceutical composition, dosage form, or hemp extract is administered at a dosage of about 0.6 mg/kg. In still another embodiment, the pharmaceutical composition, dosage form, or hemp extract is administered at a dosage of about 0.7 mg/kg. In yet another embodiment, the pharmaceutical composition, dosage form, or hemp extract is administered at a dosage of about 0.8 mg/kg. In an embodiment, the pharmaceutical composition, dosage form, or hemp extract is administered at a dosage of about 0.9 mg/kg. In another embodiment, the pharmaceutical composition, dosage form, or hemp extract is administered at a dosage of about 1 mg/kg. In yet another embodiment, the pharmaceutical composition, dosage form, or hemp extract is administered at a dosage of about 1.5 mg/kg. In still another embodiment, the pharmaceutical composition, dosage form, or hemp extract is administered at a dosage of about 2 mg/kg. In an embodiment, the pharmaceutical composition, dosage form, or hemp extract is administered at a dosage of about 3 mg/kg. In another embodiment, the pharmaceutical composition, dosage form, or hemp extract is administered at a dosage of about 4 mg/kg. In yet another embodiment, the pharmaceutical composition, dosage form, or hemp extract is administered at a dosage of about 5 mg/kg. In still another embodiment, the pharmaceutical composition, dosage form, or hemp extract is administered at a dosage of about 6 mg/kg. In an embodiment, the pharmaceutical composition, dosage form, or hemp extract is administered at a dosage of about 7 mg/kg. In another embodiment, the pharmaceutical composition, dosage form, or hemp extract is administered at a dosage of about 8 mg/kg. In yet another embodiment, the pharmaceutical composition, dosage form, or hemp extract is administered at a dosage of about 9 mg/kg. In still another embodiment, the pharmaceutical composition, dosage form, or hemp extract is administered at a dosage of about 10 mg/kg. In an embodiment, the pharmaceutical composition, dosage form, or hemp extract is administered at a dosage of about 11 mg/kg. In another embodiment, the pharmaceutical composition, dosage form, or hemp extract is administered at a dosage of about 12 mg/kg. In yet another embodiment, the pharmaceutical composition, dosage form, or hemp extract is administered at a dosage of about 13 mg/kg. In still another embodiment, the pharmaceutical composition, dosage form, or hemp extract is administered at a dosage of about 14 mg/kg. In an embodiment, the pharmaceutical composition, dosage form, or hemp extract is administered at a dosage of about 15 mg/kg. In an embodiment, the pharmaceutical composition, dosage form, or hemp extract is administered at a dosage of about 20 mg/kg. In an embodiment, the pharmaceutical composition, dosage form, or hemp extract is administered at a dosage of about 25 mg/kg. In an embodiment, the pharmaceutical composition, dosage form, or hemp extract is administered at a dosage of about 30 mg/kg. In an embodiment, the pharmaceutical composition, dosage form, or hemp extract is administered at a dosage of about 35 mg/kg. In an embodiment, the pharmaceutical composition, dosage form, or hemp extract is administered at a dosage of about 40 mg/kg. In an embodiment, the pharmaceutical composition, dosage form, or hemp extract is administered at a dosage of about 45 mg/kg. In an embodiment, the pharmaceutical composition, dosage form, or hemp extract is administered at a dosage of about 50 mg/kg. In an embodiment, the dosage is given orally. In an embodiment, the dosage is given topically.

In another embodiment, the pharmaceutical composition, dosage form, or hemp extract is administered at twice the therapeutically effective dosage for one week, and then subsequently administered at a therapeutically effective dosage. In yet another embodiment, the therapeutically effective dosage is about 0.1-0.5 mg/kg. In still another embodiment, the therapeutically effective dosage is about 2 mg/kg. In an embodiment, the therapeutically effective dosage is about 8 mg/kg.

In an embodiment, the pharmaceutical composition, dosage form, or hemp extract is administered at a dosage of about 1 mg/kg for one week, and then subsequently administered at a dosage of about 0.1-0.5 mg/kg. In another embodiment, the pharmaceutical composition, dosage form, or hemp extract is administered at a dosage of about 4 mg/kg for one week, and then subsequently administered at a dosage of about 2 mg/kg.

In an embodiment, the pharmaceutical composition, dosage form, or hemp extract is administered at a dosage of about 1.0 mg/kg once daily. In an embodiment, the pharmaceutical composition, dosage form, or hemp extract is administered at a dosage of about 1.0 mg/kg twice daily. In an embodiment, the pharmaceutical composition, dosage form, or hemp extract is administered at a dosage of about 1.0 mg/kg three times daily. In an embodiment, the pharmaceutical composition, dosage form, or hemp extract is administered at a dosage of about 1.0 mg/kg four times daily.

In an embodiment, the pharmaceutical composition, dosage form, or hemp extract is administered at a dosage of about 2.0 mg/kg once daily. In an embodiment, the pharmaceutical composition, dosage form, or hemp extract is administered at a dosage of about 2.0 mg/kg twice daily. In an embodiment, the pharmaceutical composition, dosage form, or hemp extract is administered at a dosage of about 2.0 mg/kg three times daily. In an embodiment, the pharmaceutical composition, dosage form, or hemp extract is administered at a dosage of about 2.0 mg/kg four times daily.

In an embodiment, the pharmaceutical composition, dosage form, or hemp extract is administered at a dosage of about 3.0 mg/kg once daily. In an embodiment, the pharmaceutical composition, dosage form, or hemp extract is administered at a dosage of about 3.0 mg/kg twice daily. In an embodiment, the pharmaceutical composition, dosage form, or hemp extract is administered at a dosage of about 3.0 mg/kg three times daily. In an embodiment, the pharmaceutical composition, dosage form, or hemp extract is administered at a dosage of about 3.0 mg/kg four times daily.

In an embodiment, the pharmaceutical composition, dosage form, or hemp extract is administered at a dosage of about 4.0 mg/kg once daily. In an embodiment, the pharmaceutical composition, dosage form, or hemp extract is administered at a dosage of about 4.0 mg/kg twice daily. In an embodiment, the pharmaceutical composition, dosage form, or hemp extract is administered at a dosage of about 4.0 mg/kg three times daily. In an embodiment, the pharmaceutical composition, dosage form, or hemp extract is administered at a dosage of about 4.0 mg/kg four times daily. In an embodiment, the pharmaceutical composition, dosage form, or hemp extract is administered at a dosage of about 5.0 mg/kg once daily. In an embodiment, the pharmaceutical composition, dosage form, or hemp extract is administered at a dosage of about 5.0 mg/kg twice daily. In an embodiment, the pharmaceutical composition, dosage form, or hemp extract is administered at a dosage of about 5.0 mg/kg three times daily. In an embodiment, the pharmaceutical composition, dosage form, or hemp extract is administered at a dosage of about 5.0 mg/kg four times daily.

In an embodiment, the pharmaceutical composition, dosage form, or hemp extract is administered at a dosage of about 6.0 mg/kg once daily. In an embodiment, the pharmaceutical composition, dosage form, or hemp extract is administered at a dosage of about 6.0 mg/kg twice daily. In an embodiment, the pharmaceutical composition, dosage form, or hemp extract is administered at a dosage of about 6.0 mg/kg three times daily. In an embodiment, the pharmaceutical composition, dosage form, or hemp extract is administered at a dosage of about 6.0 mg/kg four times daily.

In an embodiment, the pharmaceutical composition, dosage form, or hemp extract is administered at a dosage of about 7.0 mg/kg once daily. In an embodiment, the pharmaceutical composition, dosage form, or hemp extract is administered at a dosage of about 7.0 mg/kg twice daily. In an embodiment, the pharmaceutical composition, dosage form, or hemp extract is administered at a dosage of about 7.0 mg/kg three times daily. In an embodiment, the pharmaceutical composition, dosage form, or hemp extract is administered at a dosage of about 7.0 mg/kg four times daily.

In an embodiment, the pharmaceutical composition, dosage form, or hemp extract is administered at a dosage of about 8.0 mg/kg once daily. In an embodiment, the pharmaceutical composition, dosage form, or hemp extract is administered at a dosage of about 8.0 mg/kg twice daily. In an embodiment, the pharmaceutical composition, dosage form, or hemp extract is administered at a dosage of about 8.0 mg/kg three times daily. In an embodiment, the pharmaceutical composition, dosage form, or hemp extract is administered at a dosage of about 8.0 mg/kg four times daily.

In an embodiment, the pharmaceutical composition, dosage form, or hemp extract is administered at a dosage of about 9.0 mg/kg once daily. In an embodiment, the pharmaceutical composition, dosage form, or hemp extract is administered at a dosage of about 9.0 mg/kg twice daily. In an embodiment, the pharmaceutical composition, dosage form, or hemp extract is administered at a dosage of about 9.0 mg/kg three times daily. In an embodiment, the pharmaceutical composition, dosage form, or hemp extract is administered at a dosage of about 9.0 mg/kg four times daily.

In an embodiment, the pharmaceutical composition, dosage form, or hemp extract is administered at a dosage of about 10.0 mg/kg once daily. In an embodiment, the pharmaceutical composition, dosage form, or hemp extract is administered at a dosage of about 10.0 mg/kg twice daily. In an embodiment, the pharmaceutical composition, dosage form, or hemp extract is administered at a dosage of about 10.0 mg/kg three times daily. In an embodiment, the pharmaceutical composition, dosage form, or hemp extract is administered at a dosage of about 10.0 mg/kg four times daily.

In an embodiment, the pharmaceutical composition, dosage form, or hemp extract is administered at a dosage of about 2 mg/kg twice daily.

In an embodiment, a dropperful of the pharmaceutical composition, dosage form, or hemp extract is administered to the subject. In another embodiment, 0.5 mL of the pharmaceutical composition, dosage form, or hemp extract is administered to the subject. In another embodiment, 1 mL of the pharmaceutical composition, dosage form, or hemp extract is administered to the subject. In another embodiment, 2 mL of the pharmaceutical composition, dosage form, or hemp extract is administered to the subject. In another embodiment, 3 mL of the pharmaceutical composition, dosage form, or hemp extract is administered to the subject. In another embodiment, 4 mL of the pharmaceutical composition, dosage form, or hemp extract is administered to the subject. In another embodiment, 5 mL of the pharmaceutical composition, dosage form, or hemp extract is administered to the subject. In another embodiment, 6 mL of the pharmaceutical composition, dosage form, or hemp extract is administered to the subject. In another embodiment, 7 mL of the pharmaceutical composition, dosage form, or hemp extract is administered to the subject. In another embodiment, 8 mL of the pharmaceutical composition, dosage form, or hemp extract is administered to the subject. In another embodiment, 9 mL of the pharmaceutical composition, dosage form, or hemp extract is administered to the subject. In another embodiment, 10 mL of the pharmaceutical composition, dosage form, or hemp extract is administered to the subject.

In an embodiment, the method results in a therapeutically effective median maximal serum concentration of cannabidiol (CBD). In another embodiment, the median maximal serum concentration of CBD is about 30-90 ng/mL. In another embodiment, the median maximal serum concentration of CBD is about 30 ng/mL. In another embodiment, the median maximal serum concentration of CBD is about 50 ng/mL. In another embodiment, the median maximal serum concentration of CBD is about 70 ng/mL. In another embodiment, the median maximal serum concentration of CBD is about 90 ng/mL. In another embodiment, the median maximal serum concentration of CBDis about 90-310 ng/mL. In yet another embodiment, the median maximal serum concentration of CBDis about 90 ng/mL. In still another embodiment, the median maximal serum concentration of CBDis about 100 ng/mL. In still another embodiment, the median maximal serum concentration of CBDis about 102 ng/mL. In an embodiment, the median maximal serum concentration of CBDis about 200 ng/mL. In another embodiment, the median maximal serum concentration of CBDis about 300 ng/mL. In yet another embodiment, the median maximal serum concentration of CBDis about 400 ng/mL. In still another embodiment, the median maximal serum concentration of CBDis about 500 ng/mL. In an embodiment, the median maximal serum concentration of CBDis about 590 ng/mL. In another embodiment, the median maximal serum concentration of CBDis about 600 ng/mL.

In an embodiment, the method results in a therapeutically effective median maximal serum concentration of cannabidiolic acid (CBDA). In another embodiment, the median maximal serum concentration of CBDA is about 30-90 ng/mL. In another embodiment, the median maximal serum concentration of CBDA is about 30 ng/mL. In another embodiment, the median maximal serum concentration of CBDA is about 50 ng/mL. In another embodiment, the median maximal serum concentration of CBDA is about 70 ng/mL. In another embodiment, the median maximal serum concentration of CBDA is about 90 ng/mL. In another embodiment, the median maximal serum concentration of CBDA is about 90-310 ng/mL. In yet another embodiment, the median maximal serum concentration of CBDA is about 90 ng/mL. In still another embodiment, the median maximal serum concentration of CBDA is about 100 ng/mL. In still another embodiment, the median maximal serum concentration of CBDA is about 102 ng/mL. In an embodiment, the median maximal serum concentration of CBDA is about 200 ng/mL. In another embodiment, the median maximal serum concentration of CBDA is about 300 ng/mL. In yet another embodiment, the median maximal serum concentration of CBDA is about 400 ng/mL. In still another embodiment, the median maximal serum concentration of CBDA is about 500 ng/mL. In an embodiment, the median maximal serum concentration of CBDA is about 590 ng/mL. In another embodiment, the median maximal serum concentration of CBDA is about 600 ng/mL.

In an embodiment, the method results in a therapeutically effective median maximal serum concentration of CBD and CBDA. In another embodiment, the median maximal serum concentration of CBD and CBDA is about 30-90 ng/mL. In another embodiment, the median maximal serum concentration of CBD and CBDA is about 30 ng/mL. In another embodiment, the median maximal serum concentration of CBD and CBDA is about 50 ng/mL. In another embodiment, the median maximal serum concentration of CBD and CBDA is about 70 ng/mL. In another embodiment, the median maximal serum concentration of CBD and CBDA is about 90 ng/mL. In another embodiment, the median maximal serum concentration of CBD and CBDA is about 90-310 ng/mL. In yet another embodiment, the median maximal serum concentration of CBD and CBDA is about 90 ng/mL. In still another embodiment, the median maximal serum concentration of CBD and CBDA is about 100 ng/mL. In still another embodiment, the median maximal serum concentration of CBD and CBDA is about 102 ng/mL. In an embodiment, the median maximal serum concentration of CBD and CBDA is about 200 ng/mL. In another embodiment, the median maximal serum concentration of CBD and CBDA is about 300 ng/mL. In yet another embodiment, the median maximal serum concentration of CBD and CBDA is about 400 ng/mL. In still another embodiment, the median maximal serum concentration of CBD and CBDA is about 500 ng/mL. In an embodiment, the median maximal serum concentration of CBD and CBDA is about 590 ng/mL. In another embodiment, the median maximal serum concentration of CBD and CBDA is about 600 ng/mL.

In an embodiment, the veterinary subject is canine, feline, bovine, porcine, or equine. In another embodiment, the veterinary subject is canine. In yet another embodiment, the veterinary subject is feline. In yet another embodiment, the veterinary subject is equine.

In an embodiment, the hemp extract is administered at a dosage of about 0.1-15.0 mg/kg. In another embodiment, the hemp extract is administered at a dosage of about 0.1-10.0 mg/kg. In yet another embodiment, the hemp extract is administered at a dosage of about 0.1 mg/kg. In still another embodiment, the hemp extract is administered at a dosage of about 0.2 mg/kg. In yet another embodiment, the hemp extract is administered at a dosage of about 0.3 mg/kg. In an embodiment, the hemp extract is administered at a dosage of about 0.4 mg/kg. In another embodiment, the hemp extract is administered at a dosage of about 0.5 mg/kg. In yet another embodiment, the hemp extract is administered at a dosage of about 0.6 mg/kg. In still another embodiment, the hemp extract is administered at a dosage of about 0.7 mg/kg. In yet another embodiment, the hemp extract is administered at a dosage of about 0.8 mg/kg. In an embodiment, the hemp extract is administered at a dosage of about 0.9 mg/kg. In another embodiment, the hemp extract is administered at a dosage of about 1 mg/kg. In yet another embodiment, the hemp extract is administered at a dosage of about 1.5 mg/kg. In still another embodiment, the hemp extract is administered at a dosage of about 2 mg/kg. In an embodiment, the hemp extract is administered at a dosage of about 3 mg/kg. In another embodiment, the hemp extract is administered at a dosage of about 4 mg/kg. In yet another embodiment, the hemp extract is administered at a dosage of about 5 mg/kg. In still another embodiment, the hemp extract is administered at a dosage of about 6 mg/kg. In an embodiment, the hemp extract is administered at a dosage of about 7 mg/kg. In another embodiment, the hemp extract is administered at a dosage of about 8 mg/kg. In yet another embodiment, the hemp extract is administered at a dosage of about 9 mg/kg. In still another embodiment, the hemp extract is administered at a dosage of about 10 mg/kg. In an embodiment, the hemp extract is administered at a dosage of about 11 mg/kg. In another embodiment, the hemp extract is administered at a dosage of about 12 mg/kg. In yet another embodiment, the hemp extract is administered at a dosage of about 13 mg/kg. In still another embodiment, the hemp extract is administered at a dosage of about 14 mg/kg. In an embodiment, the hemp extract is administered at a dosage of about 15 mg/kg.

In another embodiment, the hemp extract is administered at twice the therapeutically effective dosage for one week, and then subsequently administered at a therapeutically effective dosage. In yet another embodiment, the therapeutically effective dosage is about 0.1-0.5 mg/kg. In still another embodiment, the therapeutically effective dosage is about 2 mg/kg. In an embodiment, the therapeutically effective dosage is about 8 mg/kg.

In an embodiment, the hemp extract is administered at a dosage of about 1 mg/kg for one week, and then subsequently administered at a dosage of about 0.1-0.5 mg/kg. In another embodiment, the hemp extract is administered at a dosage of about 4 mg/kg for one week, and then subsequently administered at a dosage of about 2 mg/kg.

In an embodiment, the method results in a therapeutically effective median maximal serum concentration of cannabidiol. In another embodiment, the median maximal serum concentration of cannabidiol is about 90-310 ng/mL. In yet another embodiment, the median maximal serum concentration of cannabidiol is about 90 ng/mL. In still another embodiment, the median maximal serum concentration of cannabidiol is about 100 ng/mL. In still another embodiment, the median maximal serum concentration of cannabidiol is about 102 ng/mL. In an embodiment, the median maximal serum concentration of cannabidiol is about 200 ng/mL. In another embodiment, the median maximal serum concentration of cannabidiol is about 300 ng/mL. In yet another embodiment, the median maximal serum concentration of cannabidiol is about 400 ng/mL. In still another embodiment, the median maximal serum concentration of cannabidiol is about 500 ng/mL. In an embodiment, the median maximal serum concentration of cannabidiol is about 590 ng/mL. In another embodiment, the median maximal serum concentration of cannabidiol is about 600 ng/mL.

The pharmaceutical compositions and dosage forms of the present disclosure may be administered by any convenient route, for example, by infusion or bolus injection, by absorption through epithelial or mucocutaneous linings (e.g., oral mucosa, rectal and intestinal mucosa, etc.) and may be administered together with any other therapeutic agent. Administration can be systemic or local. In an embodiment, administration is topical. In another embodiment, topical administration is used to treat local pain. In another embodiment, the local pain is joint pain. In an embodiment, the veterinary subject is an animal >100 kg (e.g., a horse, cow, or pig).

The therapeutic compositions of the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The composition and dose may vary depending upon the age, weight, and gender of a subject to be administered, target disease, disorder, syndrome, condition, route of administration, and the like. Various delivery systems are known and can be used to administer the pharmaceutical composition of the invention, e.g., encapsulation in liposomes, microparticles, microcapsules, receptor mediated endocytosis (see, e.g., Wu et al. (1987) J. Biol. Chem. 262:4429-4432). Methods of introduction include, but are not limited to, intradermal, intramuscular, intraperitoneal, intravenous, topical, transdermal, buccal, sublingual, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum, methyl cellulose, hydroxypropylmethyl-cellulose, sodium carbomethylcellulose, and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures.

The injectable preparations may include dosage forms for intravenous, subcutaneous, intracutaneous and intramuscular injections, local injection, drip infusions, etc. These injectable preparations may be prepared by methods publicly known. For example, the injectable preparations may be prepared, e.g., by dissolving, suspending or emulsifying the pharmaceutical composition, dosage form, or hemp extract in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active components may be dissolved or suspended in suitable liquids, such as fatty oils, liquid paraffin, or liquid polyethylene glycols.

Alternatively, the composition may be in a powder form for constitution before use with a suitable vehicle, e.g., sterile, pyrogen-free water. The exact formulation, route of administration and dosage may be chosen by the physician familiar with the patient's condition. (See for example Fingl, et al., 1975, in “The Pharmacological Basis of Therapeutics”, Chapter I, p. 1). Depending on the severity and responsiveness of the condition treated, dosing can also be a single administration of a slow release composition, with course of treatment lasting from several days to several weeks or until cure is effected or diminution of the disease state is achieved.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, chews, etc. In certain embodiments, for the dosages provided above, they are administered in one serving of an edible product, e.g. 1 mg/kg of hemp extract provided in an individual product.

Hemp extract, dosage forms, and pharmaceutical compositions described herein can be packaged to provide one or more doses of hemp extract per package. Any suitable type of packaging can be used, including wrappers, pouches, boxes, tubs, cans, blister packs, and bags. Such packaging is convenient and accessible to consumers, enhances the consumer's ease of use, reduces the presence of pathogens, increases shelf life, and reduces spoilage. In an embodiment, the hemp extract, dosage form, or pharmaceutical composition is packaged to provide one or more doses of hemp extract per package. In an embodiment, the package is resealable. In some embodiments, the dosage form is edible. In some embodiments, the edible dosage form is formed into a flat shape that can be more easily divided. In some embodiments, this flat shape is a disk or cookie shape. In some embodiments, the edible dosage form includes indentations to show where the edible dosage form should be divided to provide specific dosages. In some embodiments, the edible dosage form comes in multiple pieces. In some embodiments, each of the multiple pieces provides a certain dosage. In some embodiments, a package contains 1, 2, 3, 4, 5, 6, 7, 8, 9, 10, 11, 12, 13, 14, 15, 16, 17, 18, 19, 20, 25, 30, 35, 40, 45, 50, or more pieces. In some embodiments, the package is resealable. In an embodiment, one dose of hemp extract is a therapeutically effective amount. In accordance with the methods disclosed herein, pharmaceutical formulations can be administered to the patient or subject using any acceptable device or mechanism. For example, the administration can be accomplished using a syringe and needle or with a reusable pen and/or autoinjector delivery device. The methods of the present invention include the use of numerous reusable pen and/or autoinjector delivery devices to administer a pharmaceutical formulation.

In an embodiment for non-human animal administration, the term “pharmaceutical” as used herein may be replaced by “veterinary.”

In some embodiments, the patient is subject before, during, and/or after treatment to CHOP chemotherapy. CHOP chemotherapy comprises for example, without limitation, a cyclophosphamide, doxorubicin, vincristine, and steroid (e.g., prednisolone).

### EXAMPLES

While several experimental Examples are contemplated, these Examples are intended non-limiting.

### Example 1

Healthy Dogs and Cats Single-Dose Pharmacokinetics

The single-dose oral pharmacokinetics of CBD and an assessment of safety and adverse effects during 12-week administration using a hemp-based product in healthy dogs and cats were determined in this study. Eight of each species were provided a 2 mg/kg total CBD/CBDA (1 mg/kg CBD and 1 mg/kg CBDA) concentration orally twice daily for 12 weeks with screening of single-dose pharmacokinetics in six of each species. Pharmacokinetics revealed a mean maximum CBD concentration (Cmax) of 301 ng/mL and 43 ng/mL, area under the curve (AUC) of 1297 ng-h/mL and 164 ng-h/mL, and time to maximal concentration (Tmax) of 1.4 h and 2 h, for dogs and cats, respectively. Serum chemistry and CBC results showed no clinically significant alterations, however one cat showed a persistent rise in alanine aminotransferase (ALT) above the reference range for the duration of the trial. In healthy dogs and cats, an oral CBD-rich hemp supplement administered every 12 h was not detrimental based on CBC or biochemistry values. Cats do appear to absorb or eliminate CBD differently than dogs, showing lower serum concentrations and adverse effects of excessive licking and head-shaking during oil administration. More details about this study can be found in “Deabold, K. A.; Schwark, W. S.; Wolf, L.; Wakshlag, J. J., Single Dose Pharmacokinetics and Preliminary Safety Assessment with Use of CBD-Rich Hemp Nutraceutical in Healthy Dogs and Cats, Animals 2019, 9, 832, DOI 10.3390/ani9100832,” which is incorporated herein by reference in its entirety.

### Example 2

Pharmacokinetics of Oral and Transdermal Administration of Hemp Oil in Dogs

The pharmacokinetics of cannabidiol (CBD), cannabidiolic acid (CBDA), Δ9-tetrahydrocannabinol (THC), and tetrahydrocannabinolic acid (THCA) on three oral forms of CBD-rich hemp extract that contained near equal amounts of CBD and CBDA, and minor amounts (<0.3% by weight) of THC and THCA in dogs were studied. The metabolized psychoactive component of THC, 11-hydroxy-Δ9-tetrahydrocannabinol (11-OH-THC) and CBD metabolites 7-hydroxycannabidiol (7-OH-CBD) and 7-nor-7-carboxycannabidiol (7-COOH-CBD) were also assessed to better understand the pharmacokinetic differences between three formulations regarding THC and CBD, and their metabolism. Six purpose-bred female beagles were utilized for study purposes, each having an initial 7-point, 24-h pharmacokinetic study performed using a dose of 2 mg/kg body weight of CBD/CBDA (˜1 mg/kg CBD and ˜1 mg/kg CBDA). Dogs were then dosed every 12 h for 2 weeks and had further serum analyses at weeks 1 and 2, 6 h after the morning dose to assess serum cannabinoids. Serum was analyzed for each cannabinoid or cannabinoid metabolite using liquid chromatography and tandem mass spectroscopy (LC-MS/MS). Regardless of the form provided (1, 2, or 3) the 24-h pharmacokinetics for CBD, CBDA, and THCA were similar, with only Form 2 generating enough data above the lower limit of quantitation to assess pharmacokinetics of THC. CBDA and THCA concentrations were 2- to 3-fold higher than CBD and THC concentrations, respectively. The 1- and 2-week steady-state concentrations were not significantly different between the two oils or the soft chew forms. CBDA concentrations were statistically higher with Form 2 than the other forms, showing superior absorption/retention of CBDA. Furthermore, Form 1 showed less THCA retention than either the soft chew Form 3 or Form 2 at weeks 1 and 2. THC was below the quantitation limit of the assay for nearly all samples. Overall, these findings suggest CBDA and THCA are absorbed or eliminated differently than CBD or THC, respectively, and that a partial lecithin base provides superior absorption and/or retention of CBDA and THCA. More details about this study can be found in “Wakshlag J J, Schwark W S, Deabold K A, Talsma B N, Cital S, Lyubimov A, Iqbal A and Zakharov A, Pharmacokinetics of Cannabidiol, Cannabidiolic Acid, Δ9-Tetrahydrocannabinol, Tetrahydrocannabinolic Acid and Related Metabolites in Canine Serum After Dosing With Three Oral Forms of Hemp Extract, 2020, Front. Vet. Sci. 7:505. DOI 10.3389/fvets.2020.00505,” which is incorporated herein by reference in its entirety. The pharmacokinetics of transdermal administration of a CBD containing base in beagles were also studied, wherein the transdermal preparation was made with a commercially available carrier (Pencream). The base was at 70 mg (CBD/CBDA) which was applied topically on the inner pinnae twice daily.

### Example 3

Studies in Cynomolgus Macaques

This study assesses the pharmacokinetics and utility of a proprietary hemp oil comprising a mix of cannabinoids with about 90% of the mix being CBD and CBDA in the management of idiopathic diarrhea in Cynomolgus Macaques.

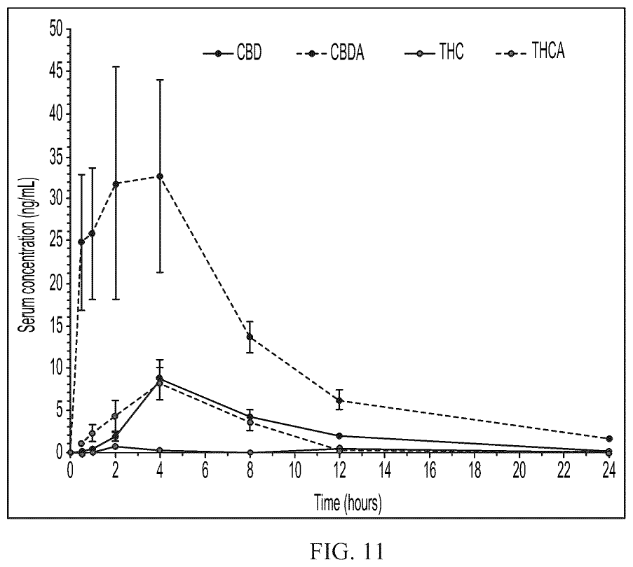

In one experiment, a population of 4 macaques was assessed for pharmacokinetic characteristics. Each macaque was given a single oral dose of 2 mg/kg hemp oil via a gummy treat. FIG. 11 shows serum levels of CBDA (upper/blue broken line), CBD (upper/blue solid line), THCA (lower/green broken line), and THC (lower/green solid line), at the indicated time points. It is noted that acidic forms of the cannabinoids appear to be better absorbed. Additionally, CBDA and CBD were present in approximately equal proportions in the hemp oil.

In another experiment, 9 macaques diagnosed with idiopathic diarrhea received the proprietary hemp oil hidden in marshmallows at a dose of 2 mg/kg once every 12 hours. This treatment was in addition to typical treatments provided to the macaques in the colony that were diagnosed with idiopathic diarrhea. Macaques that received both the hemp oil treatment and the typical treatments, compared to historical data for macaques that received only the typical treatments, showed a 33% decrease in hospital time (p=0.1).

In another experiment, 15 macaques (7 female, 8 male) were given a single dose of 2, 4, or 8 mg of hemp extract. Macaques were bled at zero, 0.5, 1, 2, 4, 8, 12, 24, 168 (7 days), and 336 hours (14 days) post dosing. Data on various cannabinoids and cannabinoid metabolites in macaque sera were collected and analyzed via targeted mass spectrometry and results are shown in Table 1, below. Animals 1, 2, 5, 7, 8, 9, 12, and 14 received the hemp extract in an oil dosage form. Animals 3, 4, 6, 10, 11, 13, and 15 received the hemp extract in a gummy dosage form.

### Example 4

Noise Aversion Treatment in Dogs

This study assesses the efficacy in the treatment of noise aversion in dogs with proprietary hemp oil.

### Method

In this study, a population of 15 canines is assessed. Patients are receiving a hemp oil based chew formulation at a dose of 4 mg/kg once2 hours before the event for a duration of 1 day. The proprietary hemp oil comprises a mix of cannabinoids with about 90% of the mix being CBD and CBDA. The study has a cross-over design, includes a washout period of 3 days and is double blinded.

The inclusion criteria include dogs being manageable and cooperative with study procedures. The dog must also react with at least 3 signs of fear to an audio recording of fireworks, storm, or thunder (Day 1) and scores at least a 30 on the Lincoln Sound Sensitivity Scale. The owner must be able to connect for video conferencing. Diagnosis of Noise Aversion must be established (storm phobia, noise phobia). The allowed medications include heartworm prevention (topical and oral), anti-parasitic treatments, vaccinations, ocular medications (including corticosteroids), antibiotics or antimicrobials and nonsteroidal anti-inflammatories. The owner must be able to collect saliva, and the dog must not have an history of aggression to owner.

The exclusion criteria comprise underlying diagnosis contributing to the clinical signs of noise aversion such as, but not limited to: a. current or previous urinary tract disease in the past 30 days; b. current or previous history of urolithiasis in the past 30 days; or c. diagnosed with or suspected, renal failure, diabetes mellitus, hypothyroidism, and neurologic disease. Excluded medications include general anesthesia or sedatives within 5 days of the Day 0 or use of CBD product, corticosteroids, diuretics, pheromones, tramadol, trazodone, benzodiazepines, gabapentin, opioids, hormones or antihistamines within 7 days of the Day 0 study visit or at any time during the study. Excluded medications include buspirone, monoamine oxidase inhibitors, serotonin reuptake inhibitors, serotoninergic medications otherwise not listed here, tricyclic antidepressants, serotonin norepinephrine reuptake inhibitors and serotonin reuptake inhibitors/antagonists within 30 days of Day 0. Excluded supplements and diets include those containing psychoactive ingredients such as but not exclusively 1-theanine, magnolia, phellodendron, alpha-casosepine and Shen Calmer within 7 days of Day 0.

The daily protocol is the following. Day 1: Three-minute acclimation period where the dog is with the pet owner in the clinical setting with the research coordinator. The only handling permitted is typical petting. Saliva is collected for cortisol measurement within 3 hours of waking with no stressful event present. Day 4: The proprietary hemp oil or placebo is administered orally. After two hours there is a three-minute acclimation period where the dog is with the pet owner in the clinical setting with the research coordinator. The only handling permitted is typical petting. Dogs are exposed to thunderstorm storm audio for 3 minutes. Saliva is collected for cortisol measurement. Day 7: The proprietary hemp oil or placebo is administered orally. After two hours there is a three-minute acclimation period where the dog is with the pet owner in the clinical setting with the research coordinator. The only handling permitted is typical petting. Dog is exposed to thunderstorm audio for 3 minutes. Saliva is collected for cortisol measurement.

### Example 5

Safety and Efficacy of Administration of Hemp Oil in Pet Birds

This study assesses the safety and efficacy in management of refractory feather plucking in psittacine pet birds.

### Method

In this study, a population of 24 birds is assessed. Patients receive a proprietary hemp oil at a dose of 15 mg/kg every 12 hours for a period of 3 months. The proprietary hemp oil comprises a mix of cannabinoids with about 90% of the mix being CBD and CBDA.

Included in this study are birds with plucking behaviors with no identified underlying medical reason as ruled out by radiographs, fecal direct and blood tests; birds with identified and medically managed diseases where there has been no clinical improvement; and birds that can have routine blood samples collected monthly to assess hematology serum chemistry.

Excluded from this study are birds less than 175 grams, birds presenting any identified medical condition associated with feather plucking that is not being treated or where clients are not following veterinary recommendations, and birds exposed to stressful and unhealthy environmental conditions typically associated with feather plucking.

Eligible birds have blood sampled for hematology and chemistry and aliquoted (200 μL) for HPLC-MS method development.

### Example 6

High-grade Lymphoma Treatment with CBD/CBDA in Dogs

This study assesses the quality of life in dogs with intermediate to high-grade lymphoma treated with a standardized CBD+CBDA protocol.

### Method

In this study, a population of 30 dogs is assessed. The study is blinded and the patients with cytologically diagnosed multicentric large cell lymphoma are randomized to one of treatment two groups, (A) prednisone alone and (B) prednisone plus CBD oil (5 mg/kg mix of CBD/CBDA as soft gel). The dosages are the following: (A) prednisone alone (2 mg/kg, PO, q24h for a week; then 1 mg/kg, PO, q48h), and (B) prednisone (2 mg/kg, PO, q24h for a week; then 1 mg/kg, PO, q48h), plus CBD oil (5 mg/kg). Patients are evaluated at days 0, 21 and 42 by a clinician. During each visit, a physical exam is done and the following measurements are performed: lymph node, CBC, chemistry profile, and UA. Half mL of serum is saved after each visit and stored at about −20 degrees C. for future CBD/CBA analysis. Patients are also evaluated at days 0, 21 and 42 by the owners to evaluate quality of life using a Quality of Life (QOL) questionnaires.

The following diagnostics on admission are performed: a physical exam, a complete blood count, a chemistry profile, an urinalysis, a lymph node measurement, and an immunophenotyping.

The following inclusion and exclusion criteria are applied: 1. dogs with cytologic or histopathologic diagnosis of intermediate to high-grade (intermediate to large-cell) peripheral lymphoma are included; 2. dogs with estimated life expectancy without any interventional therapy of ≥3 days are included; 3. dogs with known or suspected gastrointestinal involvement are excluded; 4. dogs with known or suspected cutaneous involvement are excluded; 5. dogs with known or suspected internal disease (liver/spleen) only (without peripheral lymph node involvement) are excluded; 6. dogs with clinically relevant comorbidities and small cell lymphoma are excluded; and 7. dogs receiving chemotherapy, L-asparaginase AND/OR corticosteroids prior to enrollment are excluded. No corticosteroid therapy (oral and/or topical) is allowed within the 2 months prior to enrollment.

The following four responses are evaluated in patients: (1) Complete Remission (CR), defined as complete disappearance of all evidence of disease; (2) Partial Remission (PR), defined as at least 30% decrease in the Mean Sum LD (one dimension) of target lesions; (3) Progressive Disease (PD), defined as an increase in at least 20% in the Mean Sum LD of target lesions; and (4) Stable Disease (SD), defined as neither sufficient decrease to qualify as PR nor sufficient increase to qualify as PD. Patients that experience CR, PR, or SD continue receiving treatment until PD develops. Disease free interval (DFI), if CR is achieved, and/or median survival times (MST) are compared using Kaplan Meier survival curves. The following two exit criteria are applied in this study: (1) a PD for over 10 days; and (2) a development of cancer associated clinical signs or unacceptable toxicities, as determined by the owner/guardian or veterinarian.

All cases are reviewed by the same clinical pathologist. For patients with cytology, histopathology and/or immunophenotyping, 3-6 lymph node aspirates are collected per case on positively charged slides. Slides from a single node are preferably obtained. Slides from enrolled cases are labeled with patient name, lymph node site and collection date. Slides are then placed into slide container and saved to be sent to the lab (EVP) once every two months.

Quality of Life (QOL) questionnaires are used by the owners to assess the condition of the patients. The ‘Canine Cancer Treatment Survey’ document (pdf) is provided to the dog owner. The Baseline Form (Day 0) is completed by the dog owner at time of enrollment and submitted to the Iowa State. The subsequent QOL questionnaires are completed by the Iowa State personnel via phone with the dog owner on Day 7, Day 14 and then monthly thereafter. The conclusion QOL questionnaire is completed by Iowa State personnel after the dog dies or is euthanized. The dog owner expects that the Iowa State personnel call for updates and QOL survey completion based on the timeline outlined.

### Example 7

Treatment of Skin Wounds in Dogs with CBG/CBGA

This study assesses the effect of topical CBG/CBGA in wound healing and topical CBD/CBG pyoderma models in dogs.

### Method

In this study, a population of 6 dogs is assessed. The patients receive 20 mg of CBG/CBGA or vehicle control every 12 hours to the areas (one side treated, one side vehicle control) and lesions are photographed every 2 days until resolved or necessary closure at 2 weeks. The study is blinded and includes a 2-week washout period. The second phase will be a 30 mg CBD/CBG application to areas of induced pyoderma. The dogs are screened for any dermatologic abnormalities and have physical exams performed.

During phase 1, the lesions are photographed every 2 days until resolved or necessary closure at 2 weeks. Visual assessment of the lesions is performed daily by a clinician blinded to the treatment. The images are assessed for granulation and re-epithelialization. At the end of study, a skin biopsy is collected for histopathology as well as aerobic and anaerobic culture, and a complete blood count (CBC), blood chemistry panel, and urinalysis are performed to assess any systemic effects of treatment. Blood cannabinoid levels are also assessed.

During phase 2, the dogs have an area just above the shoulder shaved bilaterally. The outer skin layer of this area is mechanically disrupted and a staphylococcus inoculum is applied for three days to induce a superficial pyoderma. Once pyoderma has been induced (confirmed by visual inspection and impression cytology; surface aerobic bacterial culture also collected), areas are then treated with either vehicle control or 40 mg CBD/CBG. Pyoderma is assessed by a blinded clinician via daily clinical lesion scoring and skin impression cytology every 2 days until resolution. Photographs of lesions are taken as well. A skin biopsy for histopathology and superficial swab for aerobic skin culture are collected at the end of the study at resolution or at conclusion after 2 weeks. Dogs have a CBC, blood chemistry panel, and urinalysis performed to assess any systemic effects of treatment. The blood cannabinoid levels are also assessed.

### Example 8

Pharmacokinetics of Hemp Oil Treatment in Horses

This study assesses the pharmacokinetics of a proprietary hemp oil in horses. The proprietary hemp oil comprises a mix of cannabinoids with about 90% of the mix being CBD or CBDA. In this study, a population of 8 horses is assessed at a dose of 2 mg/kg with. Patients receive the proprietary hemp oil (70 mg/mL) at a dose of 12 mg/kg once every 12 hours for a period The study includes a 2-week washout period between randomized CBD or CBDA treatments. Serum samples collection times are the following: Day 1: 0, 1, 2, 4, 12, and 24 hours; Day 7: 0, 1 and 2 hours; and Day 14: 0, 1 and 2 hours.

### Example 9

Pharmacokinetics and Safety Study of Cannabidiol Based Oil Treatment in Horses

This study assesses the pharmacokinetic and safety of a proprietary hemp oil in horses.

### Method

In this study, a population of 7 horses is assessed. Patients receive CBD or CBDA isolate oils at a dose of 1 mg/kg of CBD or CBDA once every 12 hours for 6 weeks. The study has a cross-over design and includes a washout period of 2 weeks. The horse selection is done based on previously diagnosed chronic musculoskeletal pathologies.

Specific clinical and laboratory assessments are conducted on each horse to meet the following 3 aims: (1) Pharmacokinetics: blood samples are obtained from each horse at time points (0, 15 minutes, 30 minutes, 1 hour, 2 hours, 4 hours, 8 hours, 12 hours, 24 hours, and 1 week, 2 weeks, 4 weeks, 6 weeks of each oil administration period). Serum is stored at −80° C. for subsequent cannabinoid concentration assessment. (2) Safety: cannabinoid oil safety is evaluated based on: 2a. Clinical examination: performed daily for the 12 weeks administration period, to rule out psychotropic effects and evaluate the development of signs suggestive of complications. 2b. Diagnostic testing to assess body systems function, and changes in inflammatory and metabolic status: bloodwork includes complete blood count (CBC), serum chemistry panel, metabolic panel (baseline cortisol, ACTH, insulin, leptin) and a cytokine panel. Blood is collected and analyzed at time points (0, 2w, 6w, 10w, and 14w post-enrollment). 2c. Hepatic ultrasonographic and histopathologic evaluation: transcutaneous ultrasonographic evaluation is performed at the beginning and end of the study to assess eventual changes in the liver structure and echogenicity, and identify the location for a liver biopsy. The biopsy of the liver is obtained transcutaneously, under ultrasound guidance, using a tru-cut device. The liver biopsy samples performed at the start (week 0) and end of the study period (week 14). (3) Efficacy: clinical response to administration of CBD, and specifically its effect on chronic pain is evaluated using standardized objective pain scoring systems and lameness evaluations utilizing a commercially available inertial sensor system (Equinosis Q™ with Lameness Locator® biomechanical platform [Columbia, Missouri, USA]), which provides an objective measurement of soundness. After recording the baseline data at time 0, before CBD administration, the pain scoring system is applied daily during the 14 weeks, while the lameness is graded and recorded through the lameness locator system on a weekly basis.

### Example 10

Pharmacokinetics of Hemp Oil Treatment in Horses

This study assessed the enteral pharmacokinetic and gastrointestinal motility of cannabidiol based oil treatment of horses.

### Method

In this study, a population of 8 horses was assessed. Patients received a single dose of proprietary hemp oil (70 mg/mL) at a dose of either 2 or 8 mg/kg once. The proprietary hemp oil comprises a mix of cannabinoids with about 90% of the mix being CBD and CBDA. The study includes a washout period of 2 weeks and healthy adult horses with a body condition score of 3-7 out of 9. One dose (either 2 mg/kg or 8 mg/kg) was given before the washout period. After the washout period the second dose, comprising the dose not yet administered to the horse, was given. The following parameters were calculated: plasma concentration of cannabidiol (and other cannabinoids) at 0, 0.5, 1, 1.5, 2, 3, 4, 8, 12, 24, and 48 hours after oral dosing of the hemp oil at 2 mg/kg CBD and CBDA; plasma concentration of cannabidiol at 0, 0.5, 1, 1.5, 2, 3, 4, 8, 12, 24, and 48 hours after oral dosing of 8 mg/kg CBD and CBDA; physical exam findings, including all identified clinical abnormalities; neurologic exam findings and video recordings; weight of feces produced every 6 hours; and the number of barium balls excreted in feces every 6 hours. From the collected data, the following parameters are calculated: Tmax: Time to maximum measured plasma concentration; Cmax: Maximum measured plasma concentration; t0.5: Time for the measured plasma concentration to decrease by half; AUC: The area under the plasma concentration curve; K: First-order elimination rate constant; kilograms of feces produced per hour; and number of barium balls per kilogram of feces produced.

Tables 2 and 3, below, shows pharmacokinetic data for 8 different horses that were given 2 mg/kg and 8 mg/kg doses of the hemp oil. Blood samples were taken from each horse at the indicated time points and subjected to targeted mass spectrometry in order to determine the serum concentration of the indicated cannabinoid compounds and metabolites.

TABLE 2
Serum cannabinoid levels in horses after a 2 mg/kg dose of hemp oil

| | | | | | | | | | | | | | |
|---|---|---|---|---|---|---|---|---|---|---|---|---|---|
| LLOQ | 1 | 1 | 1 | 0.25 | 0.5 | 1 | 2.5 | 1 | 1 | 10 | 2.5 | 1 | 1 |
| ULOQ | 1000 | 1000 | 1000 | 1000 | 1000 | 1000 | 1000 | 1000 | 2500 | 1000 | 1000 | 500 | 500 |

| | | | | | |
|---|---|---|---|---|---|
| CBD | CBDA | THC | THCA | CBG | CBGA |

MRM:

| | | | | | |
|---|---|---|---|---|---|
| 315 > | 359 > | 315 > | 357 > | 317 > | 361 > |
| 193 (+) | 219 (+) | 193 (+) | 313 (−) | 193 (+) | 219 (+) |

RT:

| | | | | | | |
|---|---|---|---|---|---|---|
| Time | 4.7 | 4.35 | 5.8 | 6.3 | 4.6 | 4.55 |

| | | | |
|---|---|---|---|
| DOSE | Horse | Point | IS: |

| (mg/kg) | ID | (h) | CBD-d3 | CBD-d3 | THC-d3 | THCA-d3 | CBD-d3 | CBD-d3 |
|---|---|---|---|---|---|---|---|---|
| 2 | Hank | 0 | BQL | BQL | BQL | BQL | BQL | BQL |
| 2 | Amy | 0 | BQL | BQL | BQL | BQL | BQL | BQL |
| 2 | Pepi | 0 | BQL | BQL | BQL | BQL | BQL | BQL |
| 2 | Lassi | 0 | BQL | BQL | BQL | BQL | BQL | BQL |
| 2 | Yetti | 0 | BQL | BQL | BQL | BQL | BQL | BQL |
| 2 | Amelia | 0 | BQL | BQL | BQL | BQL | BQL | BQL |
| 2 | Ivy | 0 | BQL | BQL | BQL | BQL | BQL | BQL |
| 2 | Tabi | 0 | BQL | BQL | BQL | BQL | BQL | BQL |
| | | MEAN: | N/A | N/A | N/A | N/A | N/A | N/A |
| 2 | Hank | 0.5 | BQL | 15.67 | BQL | 4.13 | BQL | BQL |
| 2 | Amy | 0.5 | BQL | 23.61 | BQL | 6.58 | BQL | BQL |
| 2 | Pepi | 0.5 | BQL | 9.48 | BQL | 3.65 | BQL | BQL |
| 2 | Lassi | 0.5 | BQL | 25.76 | BQL | 6.79 | BQL | BQL |
| 2 | Yetti | 0.5 | 1.48 | 65.47 | BQL | 10.61 | BQL | 1.71 |
| 2 | Amelia | 0.5 | BQL | 16.21 | BQL | 3.62 | BQL | BQL |
| 2 | Ivy | 0.5 | BQL | 16.14 | BQL | 3.04 | BQL | BQL |
| 2 | Tabi | 0.5 | 1.71 | 124.83 | BQL | 20.43 | BQL | 3.47 |
| | | MEAN: | 1.59 | 37.15 | N/A | 7.36 | N/A | 2.59 |
| 2 | Hank | 1 | 1.68 | 9.25 | BQL | 6.89 | BQL | BQL |
| 2 | Amy | 1 | 1.25 | 15.32 | BQL | 6.86 | BQL | BQL |
| 2 | Pepi | 1 | 1.32 | 5.93 | BQL | 3.40 | BQL | BQL |
| 2 | Lassi | 1 | 1.23 | 18.99 | BQL | 8.19 | BQL | BQL |
| 2 | Yetti | 1 | 6.87 | 41.86 | 1.61 | 15.38 | BQL | 1.38 |
| 2 | Amelia | 1 | 2.06 | 10.93 | BQL | 5.56 | BQL | BQL |
| 2 | Ivy | 1 | 2.25 | 12.31 | BQL | 4.51 | BQL | BQL |
| 2 | Tabi | 1 | 5.36 | 49.69 | 1.05 | 23.64 | BQL | 1.89 |
| | | MEAN: | 2.75 | 20.53 | 1.33 | 9.30 | N/A | 1.64 |
| 2 | Hank | 1.5 | 2.91 | 8.22 | BQL | 5.92 | BQL | BQL |
| 2 | Amy | 1.5 | 2.15 | 15.05 | BQL | 7.20 | BQL | BQL |
| 2 | Pepi | 1.5 | BQL | 6.05 | BQL | 3.12 | BQL | BQL |
| 2 | Lassi | 1.5 | 2.13 | 18.69 | BQL | 9.06 | BQL | BQL |
| 2 | Yetti | 1.5 | 7.76 | 36.30 | 1.62 | 15.45 | BQL | 1.09 |
| 2 | Amelia | 1.5 | 3.34 | 7.79 | BQL | 5.06 | BQL | BQL |
| 2 | Ivy | 1.5 | 3.21 | 13.43 | BQL | 4.97 | BQL | BQL |
| 2 | Tabi | 1.5 | 10.30 | 37.40 | 1.65 | 22.35 | BQL | BQL |
| | | MEAN: | 4.54 | 17.87 | 1.63 | 9.14 | N/A | 1.09 |
| 2 | Hank | 2 | 1.88 | 9.48 | BQL | 5.26 | BQL | BQL |
| 2 | Amy | 2 | 2.06 | 17.89 | BQL | 8.03 | BQL | BQL |
| 2 | Pepi | 2 | BQL | 6.00 | BQL | 2.91 | BQL | BQL |
| 2 | Lassi | 2 | 2.12 | 17.35 | BQL | 9.28 | BQL | BQL |
| 2 | Yetti | 2 | 9.86 | 31.40 | 2.12 | 15.12 | BQL | BQL |
| 2 | Amelia | 2 | 4.18 | 8.50 | BQL | 4.09 | BQL | BQL |
| 2 | Ivy | 2 | 4.01 | 16.52 | BQL | 5.52 | BQL | BQL |
| 2 | Tabi | 2 | 8.28 | 31.33 | 1.62 | 18.90 | BQL | BQL |
| | | MEAN: | 4.63 | 17.31 | 1.87 | 8.64 | N/A | N/A |
| 2 | Hank | 3 | 2.59 | 8.18 | BQL | 4.83 | BQL | BQL |
| 2 | Amy | 3 | 2.89 | 18.61 | BQL | 8.42 | BQL | BQL |
| 2 | Pepi | 3 | BQL | 7.37 | BQL | 2.94 | BQL | BQL |
| 2 | Lassi | 3 | 2.19 | 16.97 | BQL | 7.72 | BQL | BQL |
| 2 | Yetti | 3 | 7.81 | 31.32 | 1.90 | 13.24 | BQL | BQL |
| 2 | Amelia | 3 | 5.21 | 9.56 | BQL | 3.28 | BQL | BQL |
| 2 | Ivy | 3 | 5.00 | 15.70 | BQL | 5.50 | BQL | BQL |
| 2 | Tabi | 3 | 7.52 | 28.01 | 1.79 | 15.48 | BQL | BQL |
| | | MEAN: | 4.75 | 16.96 | 1.84 | 7.68 | N/A | N/A |
| 2 | Hank | 4 | 1.99 | 8.30 | BQL | 4.39 | BQL | BQL |
| 2 | Amy | 4 | 2.30 | 21.04 | BQL | 8.12 | BQL | BQL |
| 2 | Pepi | 4 | 1.14 | 6.67 | BQL | 3.03 | BQL | BQL |
| 2 | Lassi | 4 | 1.89 | 15.73 | BQL | 8.16 | BQL | BQL |
| 2 | Yetti | 4 | 9.11 | 41.36 | 1.40 | 13.99 | BQL | BQL |
| 2 | Amelia | 4 | 2.92 | 8.56 | BQL | 2.43 | BQL | BQL |
| 2 | Ivy | 4 | 7.09 | 18.61 | 1.19 | 6.12 | BQL | BQL |
| 2 | Tabi | 4 | 6.69 | 25.22 | 1.45 | 12.52 | BQL | BQL |
| | | MEAN: | 4.14 | 18.19 | 1.35 | 7.34 | N/A | N/A |
| 2 | Hank | 8 | 4.04 | 10.74 | BQL | 6.55 | BQL | BQL |
| 2 | Amy | 8 | 1.92 | 18.93 | BQL | 7.41 | BQL | BQL |
| 2 | Pepi | 8 | 2.75 | 15.57 | BQL | 5.53 | BQL | BQL |
| 2 | Lassi | 8 | 1.62 | 17.69 | BQL | 8.21 | BQL | BQL |
| 2 | Yetti | 8 | 4.93 | 27.83 | 1.00 | 8.78 | BQL | BQL |
| 2 | Amelia | 8 | 4.61 | 12.65 | 1.17 | 3.44 | BQL | BQL |
| 2 | Ivy | 8 | 2.83 | 18.89 | BQL | 4.54 | BQL | BQL |
| 2 | Tabi | 8 | 2.25 | 13.05 | BQL | 5.37 | BQL | BQL |
| | | MEAN: | 3.12 | 16.92 | 1.09 | 6.23 | N/A | N/A |
| 2 | Hank | 12 | 1.01 | 8.98 | BQL | 3.80 | BQL | BQL |
| 2 | Amy | 12 | 1.25 | 16.95 | BQL | 5.49 | BQL | BQL |
| 2 | Pepi | 12 | BQL | 11.24 | BQL | 2.56 | BQL | BQL |
| 2 | Lassi | 12 | BQL | 10.32 | BQL | 4.25 | BQL | BQL |
| 2 | Yetti | 12 | 1.84 | 23.37 | BQL | 6.05 | BQL | BQL |
| 2 | Amelia | 12 | 0.97 | 10.99 | BQL | 1.60 | BQL | BQL |
| 2 | Ivy | 12 | 1.87 | 20.14 | BQL | 3.73 | BQL | BQL |
| 2 | Tabi | 12 | 2.04 | 13.17 | BQL | 4.97 | BQL | BQL |
| | | MEAN: | 1.50 | 14.40 | N/A | 4.06 | N/A | N/A |
| 2 | Hank | 24 | BQL | 3.34 | BQL | 1.62 | BQL | BQL |
| 2 | Amy | 24 | BQL | 8.25 | BQL | 3.38 | BQL | BQL |
| 2 | Pepi | 24 | BQL | 8.73 | BQL | 1.78 | BQL | BQL |
| 2 | Lassi | 24 | BQL | 3.49 | BQL | 1.41 | BQL | BQL |
| 2 | Yetti | 24 | BQL | 9.60 | BQL | 2.95 | BQL | BQL |
| 2 | Amelia | 24 | BQL | 4.06 | BQL | 0.68 | BQL | BQL |
| 2 | Ivy | 24 | BQL | 10.14 | BQL | 2.43 | BQL | BQL |
| 2 | Tabi | 24 | BQL | 9.04 | BQL | 3.17 | BQL | BQL |
| | | MEAN: | N/A | 7.08 | N/A | 2.18 | N/A | N/A |
| 2 | Hank | 48 | BQL | BQL | BQL | BQL | BQL | BQL |
| 2 | Amy | 48 | BQL | BQL | BQL | BQL | BQL | BQL |
| 2 | Pepi | 48 | BQL | BQL | BQL | BQL | BQL | BQL |
| 2 | Lassi | 48 | BQL | BQL | BQL | BQL | BQL | BQL |
| 2 | Yetti | 48 | BQL | BQL | BQL | BQL | BQL | BQL |
| 2 | Amelia | 48 | BQL | BQL | BQL | BQL | BQL | BQL |
| 2 | Ivy | 48 | BQL | BQL | BQL | BQL | BQL | BQL |
| 2 | Tabi | 48 | BQL | 1.08 | BQL | 0.62 | BQL | BQL |
| | | MEAN: | N/A | 1.08 | N/A | 0.62 | N/A | N/A |

| | | | | 7- | | | | |
|---|---|---|---|---|---|---|---|---|
| | | | | COOH- | 7-OH- | 11-OH- | COOH- | COOH- |
| | | CBC | CBN | CBD | CBD | THC | THC | THC-Glu |
| | | MRM: | | | | | | |
| | | 315 > | 311 > | 345 > | 331 > | 331 > | 345 > | 521 > |
| | | 193 (+) | 223 (+) | 299 (+) | 201 (+) | 201 (+) | 299 (+) | 299 (+) |
| | | RT: | | | | | | |
| | | 6.15 | 5.4 | 2.35 | 2.5 | 3.6 | 3.7 | 2.15 |
| | | IS: | | | | | | |
| | | | | 7- | | | | COOH- |
| DOSE | Horse | | | COOH- | 7-OH- | 11-OH- | COOH- | THC- |
| (mg/kg) | ID | THC-d3 | CBD-d3 | CBD-d3 | CBD-d5 | THC-d3 | THC-d9 | Glu-d3 |
| 2 | Hank | BQL | BQL | BQL | BQL | BQL | BQL | BQL |
| 2 | Amy | BQL | BQL | BQL | BQL | BQL | BQL | BQL |
| 2 | Pepi | BQL | BQL | BQL | BQL | BQL | BQL | BQL |
| 2 | Lassi | BQL | BQL | BQL | BQL | BQL | BQL | BQL |
| 2 | Yetti | BQL | BQL | BQL | BQL | BQL | BQL | BQL |
| 2 | Amelia | BQL | BQL | BQL | BQL | BQL | BQL | BQL |
| 2 | Ivy | BQL | BQL | BQL | BQL | BQL | BQL | BQL |
| 2 | Tabi | BQL | BQL | BQL | BQL | BQL | BQL | BQL |
| | | N/A | N/A | N/A | N/A | N/A | N/A | N/A |
| 2 | Hank | BQL | BQL | 1.10 | NC | BQL | BQL | BQL |
| 2 | Amy | BQL | BQL | 1.66 | NC | BQL | BQL | BQL |
| 2 | Pepi | BQL | BQL | 2.67 | NC | BQL | BQL | BQL |
| 2 | Lassi | BQL | BQL | 1.91 | NC | BQL | BQL | BQL |
| 2 | Yetti | BQL | BQL | 4.77 | NC | BQL | BQL | BQL |
| 2 | Amelia | BQL | BQL | BQL | NC | BQL | BQL | BQL |
| 2 | Ivy | BQL | BQL | 3.24 | NC | BQL | BQL | BQL |
| 2 | Tabi | BQL | BQL | 5.09 | NC | BQL | BQL | BQL |
| | | N/A | N/A | 2.92 | N/A | N/A | N/A | N/A |
| 2 | Hank | BQL | BQL | 6.83 | NC | BQL | BQL | BQL |
| 2 | Amy | BQL | BQL | 10.54 | NC | BQL | BQL | BQL |
| 2 | Pepi | BQL | BQL | 9.65 | NC | BQL | BQL | BQL |
| 2 | Lassi | BQL | BQL | 12.77 | NC | BQL | BQL | BQL |
| 2 | Yetti | BQL | BQL | 32.08 | NC | BQL | BQL | BQL |
| 2 | Amelia | BQL | BQL | 7.80 | NC | BQL | BQL | BQL |
| 2 | Ivy | BQL | BQL | 11.25 | NC | BQL | BQL | BQL |
| 2 | Tabi | BQL | BQL | 41.73 | NC | BQL | BQL | BQL |
| | | N/A | N/A | 16.58 | N/A | N/A | N/A | N/A |
| 2 | Hank | BQL | BQL | 16.26 | NC | BQL | BQL | BQL |
| 2 | Amy | BQL | BQL | 19.90 | NC | BQL | BQL | BQL |
| 2 | Pepi | BQL | BQL | 15.46 | NC | BQL | BQL | BQL |
| 2 | Lassi | BQL | BQL | 29.05 | NC | BQL | BQL | BQL |
| 2 | Yetti | BQL | BQL | 62.39 | NC | BQL | BQL | BQL |
| 2 | Amelia | BQL | BQL | 22.37 | NC | BQL | BQL | BQL |
| 2 | Ivy | BQL | BQL | 27.88 | NC | BQL | BQL | BQL |
| 2 | Tabi | BQL | BQL | 97.25 | NC | BQL | BQL | BQL |
| | | N/A | N/A | 36.32 | N/A | N/A | N/A | N/A |
| 2 | Hank | BQL | BQL | 24.61 | NC | BQL | BQL | BQL |
| 2 | Amy | BQL | BQL | 30.74 | NC | BQL | BQL | BQL |
| 2 | Pepi | BQL | BQL | 19.50 | NC | BQL | BQL | BQL |
| 2 | Lassi | BQL | BQL | 44.05 | NC | BQL | BQL | BQL |
| 2 | Yetti | BQL | BQL | 84.42 | NC | BQL | BQL | BQL |
| 2 | Amelia | BQL | BQL | 32.50 | NC | BQL | BQL | BQL |
| 2 | Ivy | BQL | BQL | 45.76 | NC | BQL | BQL | BQL |
| 2 | Tabi | BQL | BQL | 151.14 | NC | BQL | BQL | BQL |
| | | N/A | N/A | 54.09 | N/A | N/A | N/A | N/A |
| 2 | Hank | BQL | BQL | 27.50 | NC | BQL | BQL | BQL |
| 2 | Amy | BQL | BQL | 43.07 | NC | BQL | BQL | BQL |
| 2 | Pepi | BQL | BQL | 26.67 | NC | BQL | BQL | BQL |
| 2 | Lassi | BQL | BQL | 55.75 | NC | BQL | BQL | BQL |
| 2 | Yetti | BQL | BQL | 119.78 | NC | BQL | BQL | BQL |
| 2 | Amelia | BQL | BQL | 54.51 | NC | BQL | BQL | BQL |
| 2 | Ivy | BQL | BQL | 66.55 | NC | BQL | BQL | BQL |
| 2 | Tabi | BQL | BQL | 163.68 | NC | BQL | BQL | BQL |
| | | N/A | N/A | 69.69 | N/A | N/A | N/A | N/A |
| 2 | Hank | BQL | BQL | 35.46 | NC | BQL | BQL | BQL |
| 2 | Amy | BQL | BQL | 54.62 | NC | BQL | BQL | BQL |
| 2 | Pepi | BQL | BQL | 33.68 | NC | BQL | BQL | BQL |
| 2 | Lassi | BQL | BQL | 61.30 | NC | BQL | BQL | BQL |
| 2 | Yetti | BQL | BQL | 145.45 | NC | BQL | BQL | BQL |
| 2 | Amelia | BQL | BQL | 61.26 | NC | BQL | BQL | BQL |
| 2 | Ivy | BQL | BQL | 88.88 | NC | BQL | BQL | BQL |
| 2 | Tabi | BQL | BQL | 177.11 | NC | BQL | BQL | BQL |
| | | N/A | N/A | 82.22 | N/A | N/A | N/A | N/A |
| 2 | Hank | BQL | BQL | 58.92 | NC | BQL | BQL | BQL |
| 2 | Amy | BQL | BQL | 89.95 | NC | BQL | BQL | BQL |
| 2 | Pepi | BQL | BQL | 145.93 | NC | BQL | BQL | BQL |
| 2 | Lassi | BQL | BQL | 80.00 | NC | BQL | BQL | BQL |
| 2 | Yetti | BQL | BQL | 197.63 | NC | BQL | BQL | BQL |
| 2 | Amelia | BQL | BQL | 122.68 | NC | BQL | BQL | BQL |
| 2 | Ivy | BQL | BQL | 137.12 | NC | BQL | BQL | BQL |
| 2 | Tabi | BQL | BQL | 171.12 | NC | BQL | BQL | BQL |
| | | N/A | N/A | 125.42 | N/A | N/A | N/A | N/A |
| 2 | Hank | BQL | BQL | 61.79 | NC | BQL | BQL | BQL |
| 2 | Amy | BQL | BQL | 99.02 | NC | BQL | BQL | BQL |
| 2 | Pepi | BQL | BQL | 128.17 | NC | BQL | BQL | BQL |
| 2 | Lassi | BQL | BQL | 80.56 | NC | BQL | BQL | BQL |
| 2 | Yetti | BQL | BQL | 178.79 | NC | BQL | BQL | BQL |
| 2 | Amelia | BQL | BQL | 102.54 | NC | BQL | BQL | BQL |
| 2 | Ivy | BQL | BQL | 144.75 | NC | BQL | BQL | BQL |
| 2 | Tabi | BQL | BQL | 166.86 | NC | BQL | BQL | BQL |
| | | N/A | N/A | 120.31 | N/A | N/A | N/A | N/A |
| 2 | Hank | BQL | BQL | 54.44 | NC | BQL | BQL | BQL |
| 2 | Amy | BQL | BQL | 100.10 | NC | BQL | BQL | BQL |
| 2 | Pepi | BQL | BQL | 122.59 | NC | BQL | BQL | BQL |
| 2 | Lassi | BQL | BQL | 66.25 | NC | BQL | BQL | BQL |
| 2 | Yetti | BQL | BQL | 178.13 | NC | BQL | BQL | BQL |
| 2 | Amelia | BQL | BQL | 82.46 | NC | BQL | BQL | BQL |
| 2 | Ivy | BQL | BQL | 144.34 | NC | BQL | BQL | BQL |
| 2 | Tabi | BQL | BQL | 184.92 | NC | BQL | BQL | BQL |
| | | N/A | N/A | 116.66 | N/A | N/A | N/A | N/A |
| 2 | Hank | BQL | BQL | 37.33 | NC | BQL | BQL | BQL |
| 2 | Amy | BQL | BQL | 68.33 | NC | BQL | BQL | BQL |
| 2 | Pepi | BQL | BQL | 82.78 | NC | BQL | BQL | BQL |
| 2 | Lassi | BQL | BQL | 46.70 | NC | BQL | BQL | BQL |
| 2 | Yetti | BQL | BQL | 122.19 | NC | BQL | BQL | BQL |
| 2 | Amelia | BQL | BQL | 55.84 | NC | BQL | BQL | BQL |
| 2 | Ivy | BQL | BQL | 112.45 | NC | BQL | BQL | BQL |
| 2 | Tabi | BQL | BQL | 114.97 | NC | BQL | BQL | BQL |
| | | N/A | N/A | 80.07 | N/A | N/A | N/A | N/A |

TABLE 3
Serum cannabinoid levels in horses after an 8 mg/kg dose of hemp oil

| | | | | | | | | | | | | | |
|---|---|---|---|---|---|---|---|---|---|---|---|---|---|
| LLOQ | 1 | 1 | 1 | 0.25 | 0.5 | 1 | 2.5 | 1 | 1 | 10 | 2.5 | 1 | 1 |
| ULOQ | 1000 | 1000 | 1000 | 1000 | 1000 | 1000 | 1000 | 1000 | 2500 | 1000 | 1000 | 500 | 500 |

| | | | | | |
|---|---|---|---|---|---|
| CBD | CBDA | THC | THCA | CBG | CBGA |

MRM:

| | | | | | |
|---|---|---|---|---|---|
| 315 > | 359 > | 315 > | 357 > | 317 > | 361 > |
| 193 (+) | 219 (+) | 193 (+) | 313 (−) | 193 (+) | 219 (+) |

RT:

| | | | | | | |
|---|---|---|---|---|---|---|
| Time | 4.7 | 4.35 | 5.8 | 6.3 | 4.6 | 4.55 |

| | | | |
|---|---|---|---|
| DOSE | Horse | Point | IS: |

| (mg/kg) | ID | (h) | CBD-d3 | CBD-d3 | THC-d3 | THCA-d3 | CBD-d3 | CBD-d3 |
|---|---|---|---|---|---|---|---|---|
| 8 | Hank | 0 | BQL | BQL | BQL | BQL | BQL | BQL |
| 8 | Amy | 0 | BQL | BQL | BQL | BQL | BQL | BQL |
| 8 | Pepi | 0 | BQL | BQL | BQL | BQL | BQL | BQL |
| 8 | Lassi | 0 | BQL | BQL | BQL | BQL | BQL | BQL |
| 8 | Yetti | 0 | BQL | BQL | BQL | BQL | BQL | BQL |
| 8 | Amelia | 0 | BQL | BQL | BQL | BQL | BQL | BQL |
| 8 | Ivy | 0 | BQL | BQL | BQL | BQL | BQL | BQL |
| 8 | Tabi | 0 | BQL | 3.02 | BQL | BQL | BQL | BQL |
| | | MEAN: | N/A | 3.02 | N/A | N/A | N/A | N/A |
| 8 | Hank | 0.5 | 2.19 | 382.81 | BQL | 47.19 | BQL | 9.70 |
| 8 | Amy | 0.5 | 2.81 | 241.58 | BQL | 34.79 | BQL | 5.18 |
| 8 | Pepi | 0.5 | 3.03 | 720.23 | BQL | 58.75 | BQL | 13.67 |
| 8 | Lassi | 0.5 | 4.10 | 848.89 | BQL | 66.60 | BQL | 15.72 |
| 8 | Yetti | 0.5 | NS | NS | NS | NS | NS | NS |
| 8 | Amelia | 0.5 | 1.03 | 84.13 | BQL | 13.89 | BQL | 2.94 |
| 8 | Ivy | 0.5 | BQL | 16.38 | BQL | 1.23 | BQL | BQL |
| 8 | Tabi | 0.5 | BQL | 6.16 | BQL | 0.87 | BQL | BQL |
| | | MEAN: | 2.63 | 328.60 | N/A | 31.90 | N/A | 9.44 |
| 8 | Hank | 1 | 6.38 | 227.45 | 1.36 | 72.78 | BQL | 8.99 |
| 8 | Amy | 1 | 10.41 | 161.53 | 2.02 | 48.56 | BQL | 3.52 |
| 8 | Pepi | 1 | 7.77 | 454.52 | 1.64 | 91.36 | BQL | 10.81 |
| 8 | Lassi | 1 | 11.03 | 549.98 | 1.49 | 101.27 | BQL | 10.62 |
| 8 | Yetti | 1 | 15.19 | 476.75 | 2.32 | 73.67 | BQL | 10.34 |
| 8 | Amelia | 1 | 8.33 | 55.44 | 1.37 | 26.34 | BQL | 2.41 |
| 8 | Ivy | 1 | 4.37 | 47.30 | BQL | 11.70 | BQL | 1.08 |
| 8 | Tabi | 1 | 1.23 | 21.22 | BQL | 6.29 | BQL | BQL |
| | | MEAN: | 8.09 | 249.27 | 1.70 | 54.00 | N/A | 6.82 |
| 8 | Hank | 1.5 | 12.79 | 114.27 | 2.42 | 62.51 | BQL | 3.90 |
| 8 | Amy | 1.5 | 18.38 | 129.59 | 3.26 | 49.02 | BQL | 2.31 |
| 8 | Pepi | 1.5 | 14.73 | 259.75 | 3.37 | 78.49 | BQL | 6.22 |
| 8 | Lassi | 1.5 | 25.44 | 346.97 | 4.84 | 90.05 | BQL | 6.89 |
| 8 | Yetti | 1.5 | 31.43 | 274.24 | 4.66 | 76.11 | BQL | 6.05 |
| 8 | Amelia | 1.5 | 15.68 | 32.74 | 3.07 | 19.32 | BQL | 0.96 |
| 8 | Ivy | 1.5 | 8.68 | 26.81 | 1.37 | 11.68 | BQL | BQL |
| 8 | Tabi | 1.5 | 5.02 | 26.73 | BQL | 12.59 | BQL | BQL |
| | | MEAN: | 16.52 | 151.39 | 3.29 | 49.97 | N/A | 4.39 |
| 8 | Hank | 2 | 15.46 | 98.61 | 3.29 | 52.92 | BQL | 2.60 |
| 8 | Amy | 2 | 24.83 | 168.14 | 4.02 | 54.23 | BQL | 2.05 |
| 8 | Pepi | 2 | 17.62 | 147.48 | 4.15 | 59.33 | BQL | 2.47 |
| 8 | Lassi | 2 | 28.61 | 273.18 | 5.19 | 89.31 | BQL | 4.83 |
| 8 | Yetti | 2 | 35.70 | 176.16 | 5.49 | 62.49 | BQL | 3.49 |
| 8 | Amelia | 2 | 18.71 | 25.79 | 3.58 | 15.29 | BQL | BQL |
| 8 | Ivy | 2 | 8.19 | 38.88 | 1.94 | 11.32 | BQL | BQL |
| 8 | Tabi | 2 | 8.69 | 26.47 | 1.09 | 14.02 | BQL | BQL |
| | | MEAN: | 19.72 | 119.34 | 3.59 | 44.86 | N/A | 3.09 |
| 8 | Hank | 3 | 17.66 | 78.38 | 3.12 | 44.08 | BQL | 1.84 |
| 8 | Amy | 3 | 33.51 | 164.34 | 5.06 | 59.39 | BQL | 2.19 |
| 8 | Pepi | 3 | 11.02 | 84.10 | 2.60 | 37.45 | BQL | 1.07 |
| 8 | Lassi | 3 | 34.25 | 346.82 | 5.26 | 108.78 | BQL | 6.33 |
| 8 | Yetti | 3 | 37.83 | 184.55 | 5.79 | 58.75 | BQL | 2.71 |
| 8 | Amelia | 3 | 21.39 | 34.32 | 3.83 | 14.57 | BQL | BQL |
| 8 | Ivy | 3 | 12.46 | 64.71 | 2.24 | 16.30 | BQL | 1.04 |
| 8 | Tabi | 3 | 20.42 | 118.61 | 2.31 | 35.50 | BQL | 2.05 |
| | | MEAN: | 23.57 | 134.48 | 3.78 | 46.85 | N/A | 2.46 |
| 8 | Hank | 4 | 14.63 | 81.85 | 2.61 | 40.36 | BQL | 1.53 |
| 8 | Amy | 4 | 43.60 | 206.13 | 7.19 | 76.43 | BQL | 1.65 |
| 8 | Pepi | 4 | 12.59 | 97.02 | 2.51 | 35.97 | BQL | 1.64 |
| 8 | Lassi | 4 | 52.48 | 356.78 | 8.41 | 113.30 | BQL | 5.20 |
| 8 | Yetti | 4 | 45.17 | 177.03 | 7.65 | 62.16 | BQL | 3.02 |
| 8 | Amelia | 4 | 16.19 | 36.59 | 3.38 | 12.75 | BQL | BQL |
| 8 | Ivy | 4 | 19.63 | 88.13 | 3.25 | 24.34 | BQL | 1.07 |
| 8 | Tabi | 4 | 38.69 | 218.71 | 5.02 | 77.54 | BQL | 3.94 |
| | | MEAN: | 30.37 | 157.78 | 5.00 | 55.35 | N/A | 2.58 |
| 8 | Hank | 8 | 24.19 | 144.29 | 4.40 | 51.37 | BQL | 2.36 |
| 8 | Amy | 8 | 15.80 | 138.61 | 3.57 | 40.19 | BQL | BQL |
| 8 | Pepi | 8 | 8.28 | 78.44 | 2.82 | 26.87 | BQL | BQL |
| 8 | Lassi | 8 | 24.25 | 163.66 | 4.34 | 78.49 | BQL | 1.62 |
| 8 | Yetti | 8 | 64.35 | 161.79 | 7.63 | 54.03 | BQL | 1.93 |
| 8 | Amelia | 8 | 41.71 | 140.08 | 9.41 | 34.89 | BQL | 1.08 |
| 8 | Ivy | 8 | 31.32 | 122.90 | 5.10 | 29.31 | BQL | 1.51 |
| 8 | Tabi | 8 | 39.03 | 145.04 | 5.95 | 65.57 | BQL | 1.03 |
| | | MEAN: | 31.12 | 136.85 | 5.40 | 47.59 | N/A | 1.59 |
| 8 | Hank | 12 | 25.65 | 128.65 | 6.09 | 47.70 | BQL | BQL |
| 8 | Amy | 12 | 8.00 | 120.23 | 1.38 | 29.28 | BQL | BQL |
| 8 | Pepi | 12 | 4.63 | 61.48 | 1.61 | 14.69 | BQL | BQL |
| 8 | Lassi | 12 | 11.95 | 102.32 | 2.18 | 44.30 | BQL | BQL |
| 8 | Yetti | 12 | 28.29 | 118.14 | 4.95 | 32.54 | BQL | BQL |
| 8 | Amelia | 12 | 14.30 | 76.85 | 4.13 | 15.43 | BQL | BQL |
| 8 | lvy | 12 | 34.97 | 102.34 | 6.06 | 28.55 | BQL | BQL |
| 8 | Tabi | 12 | 15.23 | 91.81 | 3.49 | 40.66 | BQL | BQL |
| | | MEAN: | 17.88 | 100.23 | 3.74 | 31.64 | N/A | N/A |
| 8 | Hank | 24 | 5.13 | 50.85 | 1.30 | 25.27 | BQL | BQL |
| 8 | Amy | 24 | 2.85 | 45.99 | BQL | 16.91 | BQL | BQL |
| 8 | Pepi | 24 | 2.84 | 34.88 | 1.08 | 9.27 | BQL | BQL |
| 8 | Lassi | 24 | 4.25 | 44.81 | 1.17 | 22.86 | BQL | BQL |
| 8 | Yetti | 24 | 10.68 | 73.71 | 1.72 | 17.88 | BQL | BQL |
| 8 | Amelia | 24 | 8.53 | 55.29 | 2.59 | 9.29 | BQL | BQL |
| 8 | Ivy | 24 | 7.63 | 68.16 | 1.49 | 14.48 | BQL | BQL |
| 8 | Tabi | 24 | 5.35 | 78.12 | 1.01 | 23.32 | BQL | BQL |
| | | MEAN: | 5.91 | 56.48 | 1.48 | 17.41 | N/A | N/A |
| 8 | Hank | 48 | BQL | 1.56 | BQL | 1.73 | BQL | BQL |
| 8 | Amy | 48 | BQL | 2.01 | BQL | 1.78 | BQL | BQL |
| 8 | Pepi | 48 | BQL | 2.42 | BQL | 0.73 | BQL | BQL |
| 8 | Lassi | 48 | BQL | 2.34 | BQL | 2.10 | BQL | BQL |
| 8 | Yetti | 48 | 1.73 | 3.90 | BQL | 1.67 | BQL | BQL |
| 8 | Amelia | 48 | 1.15 | 2.76 | BQL | 0.45 | BQL | BQL |
| 8 | Ivy | 48 | 1.11 | 4.66 | BQL | 1.35 | BQL | BQL |
| 8 | Tabi | 48 | 1.61 | 5.54 | BQL | 3.85 | BQL | BQL |
| | | MEAN: | 1.40 | 3.15 | N/A | 1.7 | N/A | N/A |

| | | | | 7- | | | | |
|---|---|---|---|---|---|---|---|---|
| | | | | COOH- | 7-OH- | 11-OH- | COOH- | COOH- |
| | | CBC | CBN | CBD | CBD | THC | THC | THC-Glu |
| | | MRM: | | | | | | |
| | | 315 > | 311 > | 345 > | 331 > | 331 > | 345 > | 521 > |
| | | 193 (+) | 223 (+) | 299 (+) | 201 (+) | 201 (+) | 299 (+) | 299 (+) |
| | | RT: | | | | | | |
| | | 6.15 | 5.4 | 2.35 | 2.5 | 3.6 | 3.7 | 2.15 |
| | | IS: | | | | | | |
| | | | | 7- | | | | COOH- |
| DOSE | Horse | | | COOH- | 7-OH- | 11-OH- | COOH- | THC- |
| (mg/kg) | ID | THC-d3 | CBD-d3 | CBD-d3 | CBD-d5 | THC-d3 | THC-d9 | Glu-d3 |
| 8 | Hank | BQL | BQL | BQL | BQL | BQL | BQL | BQL |
| 8 | Amy | BQL | BQL | BQL | BQL | BQL | BQL | BQL |
| 8 | Pepi | BQL | BQL | BQL | BQL | BQL | BQL | BQL |
| 8 | Lassi | BQL | BQL | BQL | BQL | BQL | BQL | BQL |
| 8 | Yetti | BQL | BQL | BQL | BQL | BQL | BQL | BQL |
| 8 | Amelia | BQL | BQL | BQL | BQL | BQL | BQL | BQL |
| 8 | Ivy | BQL | BQL | BQL | BQL | BQL | BQL | BQL |
| 8 | Tabi | BQL | BQL | BQL | BQL | BQL | BQL | BQL |
| | | N/A | N/A | N/A | N/A | N/A | N/A | N/A |
| 8 | Hank | BQL | BQL | 4.41 | NC | BQL | BQL | BQL |
| 8 | Amy | BQL | BQL | 5.66 | NC | BQL | BQL | BQL |
| 8 | Pepi | BQL | BQL | 5.88 | NC | BQL | BQL | BQL |
| 8 | Lassi | BQL | BQL | 7.51 | NC | BQL | BQL | BQL |
| 8 | Yetti | NS | NS | NS | NC | BQL | BQL | BQL |
| 8 | Amelia | BQL | BQL | 2.03 | NC | BQL | BQL | BQL |
| 8 | Ivy | BQL | BQL | BQL | NC | BQL | BQL | BQL |
| 8 | Tabi | BQL | BQL | BQL | NC | BQL | BQL | BQL |
| | | N/A | N/A | 5.10 | N/A | N/A | N/A | N/A |
| 8 | Hank | BQL | BQL | 33.39 | NC | BQL | BQL | BQL |
| 8 | Amy | BQL | BQL | 43.12 | NC | BQL | BQL | BQL |
| 8 | Pepi | BQL | BQL | 39.76 | NC | BQL | BQL | BQL |
| 8 | Lassi | BQL | BQL | 69.15 | NC | BQL | BQL | BQL |
| 8 | Yetti | BQL | BQL | 30.09 | NC | BQL | BQL | BQL |
| 8 | Amelia | BQL | BQL | 16.99 | NC | BQL | BQL | BQL |
| 8 | Ivy | BQL | BQL | 8.19 | NC | BQL | BQL | BQL |
| 8 | Tabi | BQL | BQL | 3.73 | NC | BQL | BQL | BQL |
| | | N/A | N/A | 30.55 | N/A | N/A | N/A | N/A |
| 8 | Hank | BQL | BQL | 83.28 | NC | BQL | BQL | BQL |
| 8 | Amy | BQL | BQL | 95.24 | NC | BQL | BQL | BQL |
| 8 | Pepi | BQL | BQL | 119.08 | NC | BQL | BQL | BQL |
| 8 | Lassi | BQL | BQL | 164.41 | NC | BQL | BQL | BQL |
| 8 | Yetti | BQL | BQL | 82.39 | NC | BQL | BQL | BQL |
| 8 | Amelia | BQL | BQL | 47.31 | NC | BQL | BQL | BQL |
| 8 | Ivy | BQL | BQL | 40.07 | NC | BQL | BQL | BQL |
| 8 | Tabi | BQL | BQL | 23.45 | NC | BQL | BQL | BQL |
| | | N/A | N/A | 81.90 | N/A | N/A | N/A | N/A |
| 8 | Hank | BQL | BQL | 125.66 | NC | BQL | BQL | BQL |
| 8 | Amy | BQL | BQL | 159.46 | NC | BQL | BQL | BQL |
| 8 | Pepi | BQL | BQL | 171.04 | NC | BQL | BQL | BQL |
| 8 | Lassi | BQL | BQL | 225.39 | NC | BQL | BQL | BQL |
| 8 | Yetti | BQL | BQL | 147.65 | NC | BQL | BQL | BQL |
| 8 | Amelia | BQL | BQL | 92.92 | NC | BQL | BQL | BQL |
| 8 | Ivy | BQL | BQL | 59.87 | NC | BQL | BQL | BQL |
| 8 | Tabi | BQL | BQL | 50.22 | NC | BQL | BQL | BQL |
| | | N/A | N/A | 129.03 | N/A | N/A | N/A | N/A |
| 8 | Hank | BQL | BQL | 178.14 | NC | BQL | BQL | BQL |
| 8 | Amy | BQL | BQL | 262.81 | NC | BQL | BQL | BQL |
| 8 | Pepi | BQL | BQL | 220.19 | NC | BQL | BQL | BQL |
| 8 | Lassi | BQL | BQL | 376.82 | NC | BQL | BQL | BQL |
| 8 | Yetti | BQL | BQL | 213.63 | NC | BQL | BQL | BQL |
| 8 | Amelia | BQL | BQL | 146.10 | NC | BQL | BQL | BQL |
| 8 | Ivy | BQL | BQL | 94.67 | NC | BQL | BQL | BQL |
| 8 | Tabi | BQL | BQL | 134.62 | NC | BQL | BQL | BQL |
| | | N/A | N/A | 203.37 | N/A | N/A | N/A | N/A |
| 8 | Hank | BQL | BQL | 219.22 | NC | BQL | BQL | BQL |
| 8 | Amy | BQL | BQL | 425.90 | NC | BQL | BQL | BQL |
| 8 | Pepi | BQL | BQL | 269.17 | NC | BQL | BQL | BQL |
| 8 | Lassi | BQL | BQL | 570.10 | NC | BQL | BQL | BQL |
| 8 | Yetti | BQL | BQL | 240.11 | NC | BQL | BQL | BQL |
| 8 | Amelia | BQL | BQL | 170.22 | NC | BQL | BQL | BQL |
| 8 | Ivy | BQL | BQL | 175.67 | NC | BQL | BQL | BQL |
| 8 | Tabi | BQL | BQL | 296.25 | NC | BQL | BQL | BQL |
| | | N/A | N/A | 295.83 | N/A | N/A | N/A | N/A |
| 8 | Hank | BQL | BQL | 388.42 | NC | BQL | BQL | BQL |
| 8 | Amy | BQL | BQL | 581.42 | NC | BQL | BQL | BQL |
| 8 | Pepi | BQL | BQL | 601.96 | NC | BQL | BQL | BQL |
| 8 | Lassi | BQL | BQL | 827.39 | NC | BQL | BQL | BQL |
| 8 | Yetti | BQL | BQL | 677.41 | NC | BQL | BQL | BQL |
| 8 | Amelia | BQL | BQL | 585.03 | NC | BQL | BQL | BQL |
| 8 | Ivy | BQL | BQL | 356.50 | NC | BQL | BQL | BQL |
| 8 | Tabi | BQL | BQL | 791.67 | NC | BQL | BQL | BQL |
| | | N/A | N/A | 601.23 | N/A | N/A | N/A | N/A |
| 8 | Hank | BQL | BQL | 720.14 | NC | BQL | BQL | BQL |
| 8 | Amy | BQL | BQL | 546.37 | NC | BQL | BQL | BQL |
| 8 | Pepi | BQL | BQL | 483.48 | NC | BQL | BQL | BQL |
| 8 | Lassi | BQL | BQL | 881.99 | NC | BQL | BQL | BQL |
| 8 | Yetti | BQL | BQL | 754.41 | NC | BQL | BQL | BQL |
| 8 | Amelia | BQL | BQL | 511.10 | NC | BQL | BQL | BQL |
| 8 | lvy | BQL | BQL | 804.58 | NC | BQL | BQL | BQL |
| 8 | Tabi | BQL | BQL | 845.18 | NC | BQL | BQL | BQL |
| | | N/A | N/A | 693.41 | N/A | N/A | N/A | N/A |
| 8 | Hank | BQL | BQL | 750.03 | NC | BQL | BQL | BQL |
| 8 | Amy | BQL | BQL | 562.59 | NC | BQL | BQL | BQL |
| 8 | Pepi | BQL | BQL | 488.17 | NC | BQL | BQL | BQL |
| 8 | Lassi | BQL | BQL | 840.47 | NC | BQL | BQL | BQL |
| 8 | Yetti | BQL | BQL | 899.56 | NC | BQL | BQL | BQL |
| 8 | Amelia | BQL | BQL | 559.71 | NC | BQL | BQL | BQL |
| 8 | Ivy | BQL | BQL | 646.28 | NC | BQL | BQL | BQL |
| 8 | Tabi | BQL | BQL | 754.59 | NC | BQL | BQL | BQL |
| | | N/A | N/A | 687.67 | N/A | N/A | N/A | N/A |
| 8 | Hank | BQL | BQL | 549.48 | NC | BQL | BQL | BQL |
| 8 | Amy | BQL | BQL | 419.11 | NC | BQL | BQL | BQL |
| 8 | Pepi | BQL | BQL | 342.05 | NC | BQL | BQL | BQL |
| 8 | Lassi | BQL | BQL | 542.02 | NC | BQL | BQL | BQL |
| 8 | Yetti | BQL | BQL | 758.37 | NC | BQL | BQL | BQL |
| 8 | Amelia | BQL | BQL | 348.17 | NC | BQL | BQL | BQL |
| 8 | Ivy | BQL | BQL | 612.90 | NC | BQL | BQL | BQL |
| 8 | Tabi | BQL | BQL | 591.88 | NC | BQL | BQL | BQL |
| | | N/A | N/A | 520.50 | N/A | N/A | N/A | N/A |

BQL = Below Quantitation Level
MRM = Multiple Reaction Monitoring
RT = Retention Time
IS = Internal Standard
NS—No sample
THCA: a putative isomer seen at RT 6.5 min in dosed animals, in addition to RT 6.3 min of ref standard.
COOH-THC: different RT in some samples (3.85 min in dosed animals vs of 3.7 min in ref standard; putative isomer? (at low levels, <5 ng/mL).
COOH-THC-Glu: different RT in some samples (2.35 min in dosed animals vs 2.15 min in ref standard; putative isomer? (at low levels, <5 ng/mL).
NC = Not Confirmed. 7-OH-CBD may be present at levels of lower end of standard curve (10-20 ng/mL), but it could not be confirmed due to matrix interference in dosed animals/insufficient sensitivity.

### Example 11

Pharmacokinetic Study of Hemp Extract Paste in Cats

This study assesses the pharmacokinetics of a hemp oil paste in cats.

### Method

In this study, a population of 10 cats is assessed. Patients receive a paste at a dose of 2 mg/kg of proprietary hemp oil once every 12 hours for about 15 days. The proprietary hemp oil comprises a mix of cannabinoids with about 90% of the mix being CBD and CBDA. Patients are healthy domestic short-hair cats, 1-8 years old, and weighing 4 to 5.5 kg.

The test utilizes 10 cats for 15 days. On Day 0, the test article is administered in the morning and food is provided shortly after. Serial blood collections for pharmacokinetic analysis are obtained from each cat over a 24-hour period. Beginning on Day 1, after the 24-hour blood collection, the test article is administered BID for 14 days. On Days 7 and 14, a six-hour blood collection is performed for pharmacokinetic analysis after the morning dose. All cats are fed the control diet only during the study. Daily observations, food consumption and weekly body weight are assessed throughout the study. Physical examinations are performed prior to study start and at study completion. Blood analysis (CBC and chemistry screen) is performed prior to study start and at the conclusion of the study.

### Example 12

Anti-inflammatory Effect of CBD/CBDA in Dogs

This study assesses the pharmacokinetics of a hemp oil chew in dogs.

### Method

In this study, a population of 8 dogs is assessed. Blood is taken from the dogs and undergoes separation of lymphocytes and neutrophils. PMA induced neutrophil chemotaxis, reactive oxygen species production and phagocytic function is assessed after 24-48 hr using in vitro assays. Incubation with 50 and 500 ng of CBD, CBDA and whole plant extract is performed. Furthermore, ConA and PHA stimulated T cell function assays are done with similar incubations examining T cell elaboration of pro-inflammatory cytokines (IL-17, IL2, IL-4 and IFN gamma) and anti-inflammatory cytokine IL-10 using ELISPOT assays over 72 hrs. Additional prostaglandin ELISAs are performed on neutrophil supernatants and in A-72 fibroblast assays after CBD, CBDA and whole plant extracts for 48 hr and subsequent 24 hr stimulation of LPS. In addition, a cytokine array of the treated A-72 fibroblast supernatants is performed by Eve Technologies. Based on the results, a second arm of the study examining in vivo effects on cells before and after treatment in dogs with chronic inflammatory conditions is assessed to further understand the effects of hemp oil on immune cell function.

### Example 13

Pharmacokinetic Study of CBG/CBGA Hemp Oil in Dogs

This study assesses the pharmacokinetics of cannabigerol (CBG) and cannabigerolic acid comprising hemp oil in dogs.

### Method

In this study, a population of 6 dogs is assessed. Patients receive a proprietary hemp oil comprising cannabigerol/cannabigerolic acid at a 2 mg/kg oral dose (approximately 1 mg of CBG and 1 mg of CBGA) in a sesame oil base, have 24 hr pharmacokinetics assessed, and then on day 2-14 are dosed every 12 hrs with serum assessments at week 1 and 2, 6 hrs after the morning dose on day 7 and day 14 of administration. Dogs are fasted during this phase and fed 2 hrs after dosing during this phase of the trial. Dogs have then a 2-week washout period and afterward undergo the same protocol with feeding ¼ can of wet food during the administration of the CBG/CBGA oil for the following two week period. Dogs have physical examinations and heart rate assessed multiple times on the first day of administration and then every 3-4 days during the trial weeks.

The daily protocol is the following. Day 1: blood draws at 0.5, 1, 2, 4, 8, 12 and 24 hrs, a physical exam, and a heart rate measurement are performed. Day 2: a blood draw, a physical exam, and a heart rate measurement are performed. Day 3: a blood heart rate measurement and a physical exam are performed. Day 7: a physical exam, a heart rate measurement, and a blood draw are performed. Day 10: a physical exam and a heart rate measurement are performed. Day 14: a physical exam, a heart rate measurement and a blood draw are performed. Day 27: a blood draw, a physical exam and a heart rate measurement are performed. Day 28: blood draws a 1, 2, 4, 8, and 12 hrs, a physical exam, and a heart rate measurement are performed. Day 29: a blood draw, a physical exam and a heart rate measurement are performed. Day 30: a physical exam and a heart rate measurement are performed. Day 35: a physical exam, a heart rate measurement and a blood draw are performed. Day 38: a physical exam and a heart rate measurement are performed. Day 42: a physical exam, a heart rate measurement and a blood draw are performed.

### Example 14

P450 and P-gp Characterization of CBD, CBDA, and Hemp Extracts in Dogs

These two studies aim to identify the main canine P450 and P-gp proteins responsible for metabolizing CBD and CBDA in the liver and determine the potential for CBD, CBDA and hemp extracts rich in CBD and CBDA to inhibit canine P450 drug metabolism.

### Method (P450 Characterization)

In this first study, an in vitro substrate depletion assay to measure the rate of metabolism of CBD and CBDA in dog liver is performed. Incubations are performed using pooled dog liver microsome fractions and P450 cofactors. Substrates remaining in the incubation are measured by HPLC-MS and compared to negative control reactions that use inactivated microsomes. A complementary study to evaluate direct conjugation by UDP-glucosyltransferases (UGTs) as an alternate (non-P450) metabolic pathway is also performed using the same approach (UGT cofactor used instead of P450 cofactor).

The rates of metabolism of CBD and CBDA by all available dog recombinant P450 enzymes are determined (8 commercial and 3 generated in the PI's lab). The assays are the same as above except recombinant P450 enzymes are used instead of liver microsomes. Since the amount of each P450 enzyme varies greatly in the liver, the results to canine liver are extrapolated using tissue specific P450 concentrations that are previously determined for each isoform.

Inhibition potency (i.e. IC50) is determined by measuring the decrement in metabolism of P450-specific marker activities in pooled dog liver microsomes with increasing concentrations of CBD, CBDA, and hemp extracts. P450-specific markers include bupropion 6-hydroxylation (CYP2B11), dextromethorphan 0-demethylation (CYP2D15) and omeprazole sulfoxidation (CYP3A12). IC50 values are determined both with and without preincubation of liver microsomes with potential inhibitor to evaluate whether there is mechanism-based time-dependent inhibition.

The following parameters are assessed: (1) the rates of CBD and CBDA metabolism by dog liver microsomes and by 11 recombinant canine P450 enzymes; (2) the predicted percent contribution of each P450 to total CBD and CBDA metabolism in dog liver; and (3) IC50 values for inhibition of CYP2B11, CYP2D15 and CYP3A12 metabolism in dog liver, both with and without inhibitor preincubation.

Partial Results

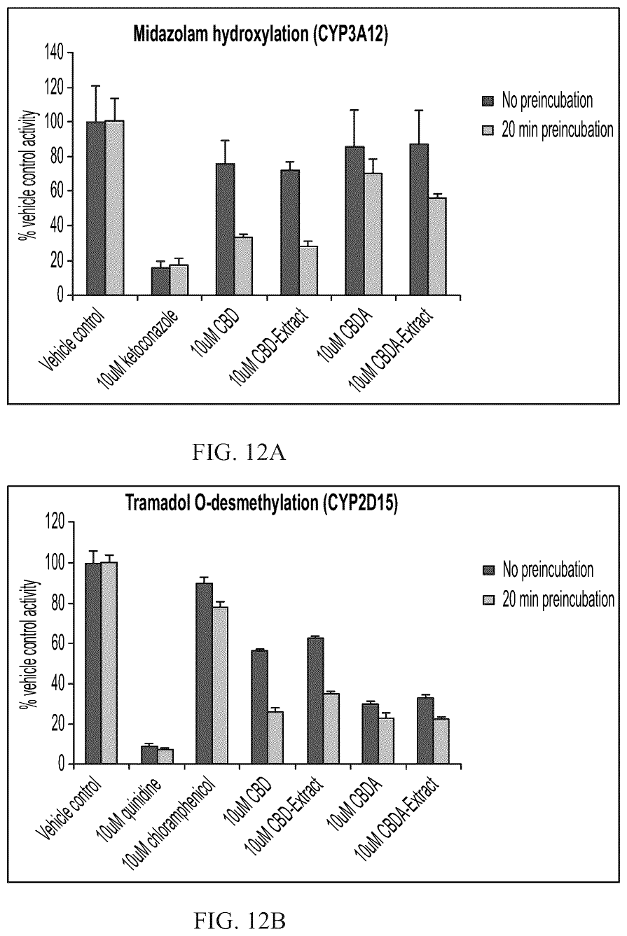

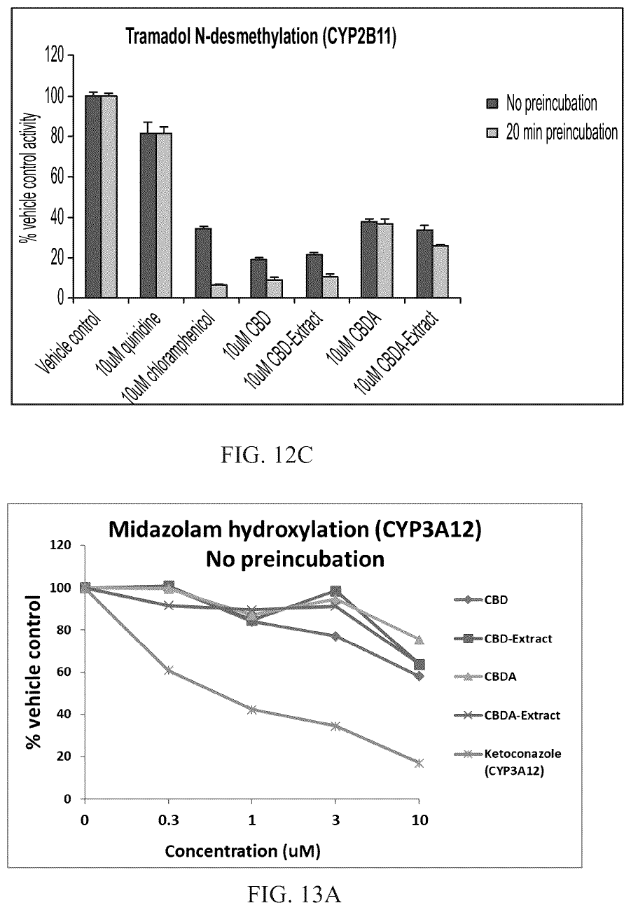

Initial results are shown in FIGS. 12-15.

The CBD and CBDA extracts yielded results that were identical to the respective pure compounds. See FIG. 12A-12C and compare “10 μM CBD” to “10μM CBD-Extract” and “10μM CBDA” to “10 μM CBDA-Extract”

CBD and CBD-extract showed time-dependent inhibition (TDI) for all P450 activities; CBDA and CBDA-extract did not show TDI (compare “No preincubation” bars (left bar, no outline) to “20 min preincubation” (right bar, with outline).

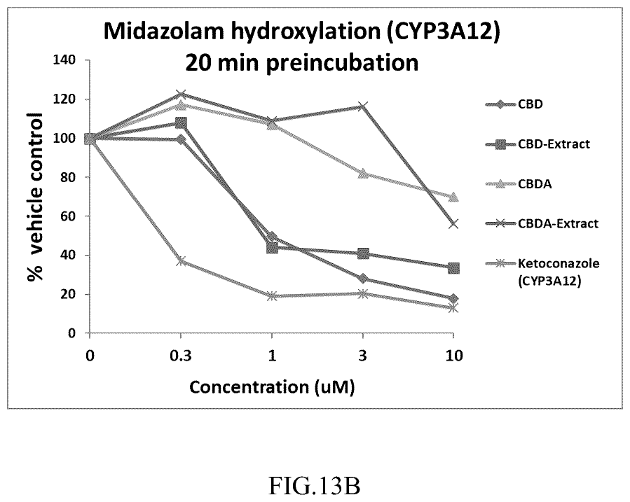

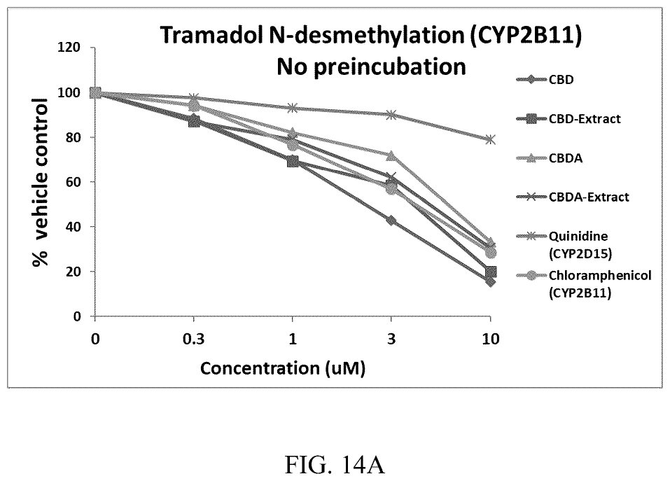

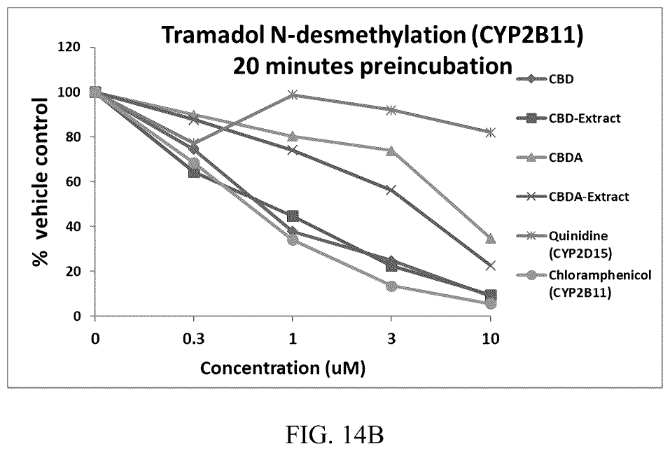

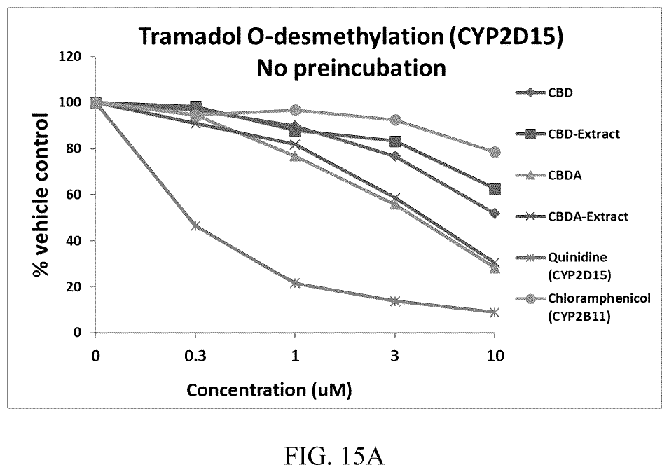

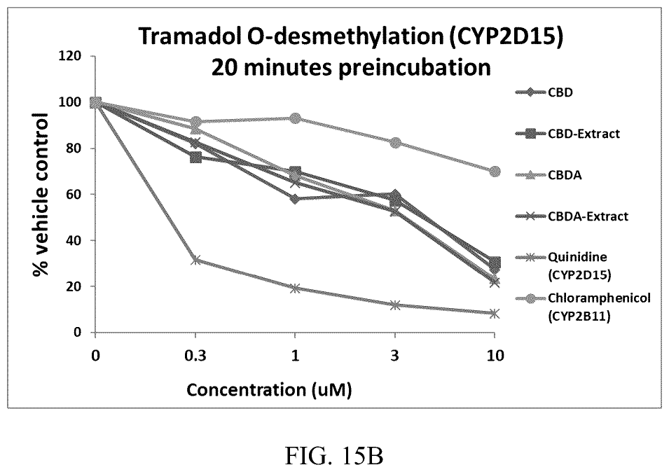

FIGS. 13-15 show activity assays for CYP3A12 (FIGS. 13A and 13B), CYP2B11 (FIGS. 14A and 14B), and CYP2D15 (FIGS. 15A and 15B). Assays using either no preincubation (FIGS. 13A, 14A, and 15A) or 20 minutes of preincubation (FIGS. 13B, 14B, and 15B) were performed. Only CBD and CBD extract showed potent inhibition (e.g., IC50 of less than 1 μM). This occurred when either CBD or CBD-extract was preincubated with microsomes and NADPH, and either CYP2B11 activity (tramadol N-demethylation, FIG. 14B) or CYP3A12 activities (midazolam hydroxylation, FIG. 13B) were measured.

CYP2D15 activities (tramadol 0-demethylation, FIGS. 15A and 15B) were much less potently inhibited (IC50>3 μM) by all compounds regardless of preincubation.

5. Overall, these results suggest that CBD and CBD-extract could have clinically significant interactions with CYP2B11 and CYP3A12 substrates in vivo. However, these results are preliminary.

### Method (P-gp Characterization)

In this second study, a competitive P-gp substrate assay incorporating a canine P-gp expressing cell line is used to assess each test compound's ability to compete with the P-gp substrate rhodamine 123. Each concentration is tested on 3 separate days with same-day duplicates. Positive controls and negative controls are included in each run.

Results for each concentration of test compound evaluated are reported as non-P-gp substrate, weak P-gp substrate, moderate P-gp substrate, or strong P-gp substrate. The concentrations for CBD and CBDA are as follows: 10 ng/mL, 100 ng/mL and 1,000 ng/mL.

Results

A competitive P-gp substrate assay using a canine P-gp expressing cell line was used to assess CBDA and/or CBD as a substrate for canine P-glycoprotein.

The concentrations proposed for CBD and CBDA are as follows: 10 ng/mL, 100 ng/mL and 1,000 ng/mL Data for CBD at 1,000 ng/mL are shown in Table 4. The highest concentration of CBD tested does not compete with rhodamine for PGP efflux with an MFI ratio essentially identical to the negative control, meaning that CBD is not a substrate for canine PGP in this assay. Results for lower concentrations of CBD have looked appear similar, but final data has not been collected.

TABLE 4
P-glycoprotein competitive transport assay

| | MFI in MDCK | Mean MFI ratio |
|---|---|---|
| Compound Tested | PGP cells | compound:vehicle |
| Rhodamine alone | 215 | 1 |
| Loperamide (canine + | 1533 | 7.1 |
| control) | | |
| Carboplatin (canine − | 160 | 0.74 |
| control) | | |
| CBD (1000 ng/mL) | 156 | 0.73 |

### Example 15

Effect of Cannabidiol on Dogs Neoplastic Cell Proliferation and Mitogen-activated Protein Kinase Activation During Autophagy and Apoptosis

This study is to assess both the anti-proliferative and cell death response associated with in vitro treatment of canine cancer cell lines with CBD alone and combination with common chemotherapeutics and the proliferative pathways (e.g., p38, JNK, AKT and mTOR) potentially involved in the response to treatment with CBD.

### Materials and Method

1. Cannabinoids and Chemotherapeutics

CBD and its acid derivative CBDA are purchased as a 10 mg/mL and a 1 mg/mL formulation in methanol, respectively (Cayman Chemical Corporation, Ann Arbor, Michigan). A whole hemp based extract is received directly from a manufacturer with third party analysis (Proverde Laboratory, Milford, Massachusetts) revealing a product with approximately 30 mg/mL of CBD, 31 mg/mL CBDA, 1.4 mg/mL THC and 1.3 mg/mL tetrahydrocannabinoic acid (THCA) with less than 1 mg/mL of cannabigerol, cannabichromene and cannabinol, and 5.2 mg/mL of complex terpenes (ElleVet Sciences, Portland, Maine) in an ethanol base. The extract is diluted to a 20 mg/mL in a 50%/50% mix of ethanol and DMSO. The final stock extract contains 20 mg/mL of cannabidiols as an equal mix of CBD (10 mg) and CBDA (10 mg) as well as 0.4 mg/mL THC, 0.4 mg THCA, 0.1 mg or less of cannabichromene (CBC) and cannabigerol (CBG) with 1.8 mg of complex terpenes. Chemotherapeutic agent doxorubicin hydrochloride (Sigma Aldrich, St. Louis, Missouri) is freshly diluted in water to a 2 mM stock solution, while vincristine sulfate (Sigma Aldrich, St. Louis, Missouri) is prepared as a stock solution of 10 μM in sterile water before utilizing in cell culture experiments.

2. Cell Lines and Culture Conditions

Five established canine neoplastic cell lines are obtained and used for all experiments; a cell line of epithelial mammary gland carcinoma cell line—CMT12, a B cell lymphoma lineage-17-71, and three mesenchymal osteosarcoma lines HMPOS, D17 (#CCL-183; ATCC, Manassas, Virginia) and Abrams. The Abrams cell line is validated from its original source, while the D17 cell line is a validated cell line from the American Type Culture collections. The CMT12, 17-71 and HMPOS cell lines have not been validated genetically but display cell markers and characteristics of epithelial, round and osteosarcoma cell lines, respectively. All cell lines are deemed mycoplasma free by polymerase chain reaction from the Animal Health and Diagnostic Laboratory at Cornell University.

All cells are maintained on tissue culture-treated plates (Laboratory Product Sales [LPS], Rochester, New York) with Roswell Park Memorial Institute (RPMI) medium 1640 (Invitrogen, Carlsbad, California) with 10% fetal bovine serum (FBS; Invitrogen, Carlsbad, California) and 1% antibiotic and antimycotic solution (Invitrogen, Carlsbad, California). Cell lines are grown at 37° C. and 5% CO₂ for all experiments and passage of cells, unless indicated otherwise. Canine primary dermal fibroblast (Applied Biological Materials [ABM], Richmond, BC, Canada) are propagated and kept on PriCoat T25 flasks (ABM) in Prigrow II medium (ABM) containing 10 HI-FBS and 1% penicillin/streptomycin (Invitrogen, Carlsbad, California). The dermal fibroblasts are used to determine the effects of the extract on normal cells.

3. Cannabidiols and CBD-rich Hemp 48 Hours MTT Proliferation

3-(4,5-Dimethylthaizol-2-yl)-2,5-diphenyltetrazolium bromide (MTT) assays are performed on all previously described cell lines; CMT12, HMPOS, D17, Abrams and 17-71. Cells are plated at a density of 2500 cells per well in 96-well tissue culture-treated plates (Laboratory Product Sales, Rochester, New York). Cells are treated with vehicle (methanol or ethanol/DMSO mix) or various concentrations of the CBD, CBDA or CBD-rich hemp extract ranging from 0.42 to 20 μg/mL in serial dilution for 48 hours. MTT assays are performed after 48 hours of treatment by adding 20 μL of MTT dye (0.7 μM filtered 5 mg/mL in phosphate-buffered saline [PBS]) to each well and incubating at 37° C. in 5% CO₂ for 2 hours. The media is then aspirated, washed once with 200 μL of PBS and then solubilized in 200 μL of ethanol. Immediate analysis of the optical density of each well is performed using a spectrophotometric plate reader (Epoch; Biotek, Winooski, Vermont) at a wavelength of 570 nm as previously described (e.g., Vega-Avila E and Pugsley M K, An overview of colorimetric assay methods used to assess survival or proliferation of mammalian cells, Proc West Pharmacol Soc., 2011, 54, pp. 10-14 which is incorporated herein by reference in its entirety). The percent proliferating cells of control for each concentration is averaged and reported as a mean+/—SD from triplicate wells over three experiments.

4. Doxorubicin Cytotoxicity/Proliferation Assays

CMT12, 17-71 and D17 cells are plated at a density of 2500 cells per well in 96-well tissue culture-treated plates (Laboratory Product Sales, Rochester, New York). All cell lines are treated with identical concentrations of the pure CBD (0.34, 0.67, 1.25, 2.5, 5, 10, 20 g/mL) and various concentrations of doxorubicin or vincristine. The concentration of doxorubicin varies between cell lines in order to achieve between 20% and 80% proliferation inhibition. The cell lines are treated with serial dilutions of doxorubicin as follows; CMT12 and D17 (0.067-2 μM) and 17-71 (0.0167-0.5 μM). Methanol is used as a vehicle control for all CBD treatments and sterile water for doxorubicin and vincristine at the highest doses used to represent the vehicle control treated wells. Cells are then incubated for 48 hours prior to performing MTT assays, as previously described. Wells treated with the vehicle control are considered to represent 100% proliferating cells in triplicate over three experiments. Percent viable cells for each specific combination are averaged and reported as mean percent proliferation+/— SD for further CI evaluation.

5. Trypan Blue Exclusion Assay of Cell Viability

The trypan blue exclusion assay is performed on canine primary dermal fibroblasts (CDF) because of the slow rate of proliferation and low metabolic activity of these normal canine cells, precluding productive MTT assays. The effects of CBD treatments are compared with the results obtained on the 17-71, CMT12 and D17 cell lines for comparative purposes. For the CDF cells, applied cell extracellular matrix (ABM) is applied overnight to 24-well tissue culture-treated plates (Applied Biological Materials [ABM], Richmond, BC, Canada). For all cell lines, cells are plated at a density of 5×10³ cells per well and incubated until 60% confluent before treatment with methanol vehicle control, 3.75, 7.5, and 15 μg/mL of CBD to cells for 48 hours. Cells are then trypsinized, collected and centrifuged at 1900 g for 10 minutes. With the exception of the 17-71 cell line, cells are detached with Accumax (Invitrogen, Carlsbad, California). The cell pellet is resuspended in 0.1% trypan blue (Sigma Aldrich, St Louis, Missouri) in PBS solution, loaded on a Cell Countess disposable cell counting slide with automatic counting of all positively stained cells in the Countess II Cell Counter under identical settings for each cell line (Invitrogen; ThermoFisher Scientific, Carlsbad, California) using the same parameters for each cell line. All treatments are performed in triplicate and the percent of viable cells are averaged.

6. MTT and Trypan Blue Data Management and Calculations

Raw data from MTT proliferation assays and trypan blue exclusion assays (individual optical density of each well) are normalized to the vehicle control treatment for each cell line, considered to represent 100% proliferating cells (single or combined treatment). The percent proliferating cells is determined by comparing optical density readings or live counts, respectively, of treatment wells at each concentration compared with vehicle control wells in each cell line.

For CBD, CBDA and hemp extract concentrations needed to obtain a 50% inhibition of cell proliferation (IC50) are then calculated across experiments by Probit analysis using XLFit5 software (IDBS, Guildford, United Kingdom) for reporting the results for each cell line.

The compound interactions of chemotherapy and CBD treatment in the CI studies are calculated by multiple drug effect analysis using Compusyn software (v.2; Compusyn Inc, Paramus, New Jersey) which employs the median equation principle according to the methodology described by Chou and Talalay to determine a CI value by the Formula I (Chou T C, Drug combination studies and their synergy quantification using the Chou-Talalay method, Cancer Res., 2010, 70(2), pp. 440-446, which is incorporated herein by reference in its entirety):

$$CI = \frac{{( D )}_{1}}{{( {D}_{X} )}_{1}} + \frac{{( D )}_{2}}{{( {D}_{X} )}_{2}} + \frac{{( D )}_{1} {( D )}_{2}}{{( {D}_{X} )}_{1} {( {D}_{X} )}_{2}} \qquad ( I )$$

where (D)₁ and (D)₂ are the doses of both compounds in combination and (Dₓ)₁ and (Dₓ)₂ are the doses of each compound alone at x percent of inhibition. CI values≤0.9 indicate synergism, a CI value>0.9 and <1.1 indicates an additive effect, and CI values≥1.1 indicate antagonism.

7. Annexin V-FITC Apoptosis Assay

Apoptosis after 4 and 8 hours treatment is measured using Annexin-V staining (Invitrogen Annexin V-FITC staining kit, Carlsbad, California). Briefly, cells are detached with Accumax (Innovative Cell Technologies, San Diego, California), collected and centrifuged for 10 m at 1000 g at 4° C. The pellet is washed once with PBS before resuspension in Annexin Binding Buffer (ABB; 10 mM HEPES, 140 mM NaCl, 2.5 mM CaCl₂, pH 7.4) at a density of approximately 1×10⁶ cell/mL. Annexin V-FITC conjugate is added according to the manufacturer's suggestion to the cell suspensions and incubated for 15 minutes at room temperature. After the incubation, ABB is added to the cell suspension and kept on ice until fluorescence is measured with the BD FACScalibur flow cytometer using an argon laser (BD Biosciences, Ashland, Oregon). Ten thousand events are collected per sample. Analysis is performed with the FlowJo software (Version 10.7.1. Becton, Dickinson, Ashland, Oregon) by first gating based on the forward- and side-scatter characteristics for each cell line followed by Annexin V-FITC positive cells. Negative fluorescence controls are unstained cells. Three independent replicates are examined for each treatment.

8. Western Blot Assessment of Signalling Pathways and Autophag

Cells are plated on 100 mm tissue culture-treated plates and incubated overnight in complete medium until 60% confluency is reached. Cells are treated with methanol vehicle control or 10 μg/mL of CBD for 2, 4 or 8 hours. Cells are harvested and lysed at each time point for control and CBD treated cells using mammalian lysis buffer (25 mM Tris, 100 mM NaCl, 1 mM EDTA, 1% Triton X-100, pH 7.4), and then centrifuged for 5 minutes at 12 000 g at 4° C. The supernatant is collected and the protein concentration is determined using the Bradford assay (Coomassie-dye; ThermoFisher Scientific Pierce, Waltham, Massachusetts). Samples are equilibrated to a common volume (μg/μL) in lysis buffer and 5× laemmili loading buffer (300 mM Tris-HCl pH 6.8, 10% sodium dodecyl sulfate, 50% glycerol, 12.5% (β-mercaptoethanol, 0.025% bromophenol blue). For each protein of interest, 30 μg total protein are subjected to sodium dodecyl sulfate polyacrylamide gel electrophoresis (SDS-PAGE) on gels ranging from 6% to 15% based on the molecular weight of the protein of interest. The proteins are then transferred to 0.45 pin pore size polyvinylidene fluoride membrane (Immobilon-P Membrane, EMD Millipore, Billerica, Massachusetts) for 1 hour at 333 mA and then blocked in 5% milk in tris-buffered saline/0.05% Tween 20 solution (TBST). Membranes are incubated overnight in primary antibody solutions at a dilution of 1:1000 in TBST on a rocking platform at 4° C. Primary antibodies confirmed as cross reactive with canine cells or tissues include mouse extracellular regulated kinase (ERK) (R&D Biosciences, Boston, Massachusetts); rabbit anti-protein kinase B (AKT), Ser473 phosphorylated-AKT, stress-activated protein kinase/jun-amino-terminal kinase (SAPK/JNK), Thr183/Tyr185 phosphorylated-SAPK/JNK, mammalian target of rapamycin (mTOR), Ser2448 phosphorylated-mTOR, anti-Thr202/Tyr204 phosphorylated p44/42 MAPK (ERK1/2), anti-p38, anti-phosphorylated p38, anti-p62 and anti-LC3 A/B (Cell Signalling Technology, Danvers, Massachusetts)(e.g., Syrjä P, Anwar T, Jokinen T, Kyöstilä K, Jäderlund K H, Cozzi F, Rohdin C, Hahn K, Wohlsein P, Baumgärtner W, Henke D, Oevermann A, Sukura A, Leeb T, Lohi H, and Eskelinen E L, Basal Autophagy Is Altered in Lagotto Romagnolo Dogs with an ATG4D Mutation, Vet Pathol, 2017 November, 54(6), pp. 953-963, doi: 10.1177/0300985817712793; Gordon I K, Ye F, and Kent M, Evaluation of the mammalian target of rapamycin pathway and the effect of rapamycin on target expression and cellular proliferation in osteosarcoma cells from dogs, Amer J Vet Res, 2008, 69, pp. 1079-1084; Ikari A, Atomi K, Kinjo Y, and Sugatani J, Magnesium deprivation inhibits a MEK-ERK cascade and cell proliferation in epithelial Madin-Darby canine kidney cells, Life Sci, 2010, 86, pp. 766-773; and Levine C B, Bayle J, Biourge V, and Wakshlag J J, Effects and synergy of feed ingredients on canine neoplastic cell proliferation, BMC Vet Res, 2016, 12(1), 159, which are incorporated herein by reference in their entirety). Membranes are washed three times with TBST and incubated at room temperature for 1 hour in the corresponding secondary anti-rabbit IgG or anti-mouse IgG horseradish peroxidase-conjugated antibody at a dilution of 1:2000 (Cell Signalling Technology, Danvers, MA). Membranes are washed three times with TBST and visualized with a chemi-luminescent reagent (Clarity Western ECL Substrate; Bio-Rad, Hercules, California). Digital images are captured using an imaging system (Biospectrum 410; UVP, Upland, California or FluorChem E; Cell Biosciences, San Jose, California). Each blot is performed twice from two different experiments to confirm findings.

9. D17 and CMT12 Immunofluorescence

CMT12 and D17 adherent cell lines are split into Nunc chamber slides (ThermoFisher Scientific, Rochester, New York) and the cells that are 70% confluent are treated for 6 hours with either methanol control or 10 μg/mL of CBD. Cells are fixed with 4% paraformaldehyde for 1 hour and then permeabilized with PBS containing 0.1% Triton X-100 for 30 minutes. Cells are then washed with PBS and incubated with bovine serum albumin (Sigma Aldrich, St. Louis, Missouri) for 30 minutes and then goat anti-serum (Vector Labs, Burlingame, California) for 1 hour and then washed twice with PBS for 10 minutes while rocking at room temperature. Equal concentrations of rabbit polyclonal non-specific antibody (Vector Labs, Burlingame, California) or polyclonal rabbit-anti LC3A/B antibody (Cell Signalling, Danvers, Massachusetts) at identical concentrations as described by Syrja and colleagues are sued (Syrjä P, Anwar T, Jokinen T, Kyöstilä K, Jäderlund KH, Cozzi F, Rohdin C, Hahn K, Wohlsein P, Baumgärtner W, Henke D, Oevermann A, Sukura A, Leeb T, Lohi H, and Eskelinen E L, Basal Autophagy Is Altered in Lagotto Romagnolo Dogs with an ATG4D Mutation, Vet Pathol, 2017 November, 54(6), pp. 953-963, doi: 10.1177/0300985817712793, which is incorporated herein by reference in its entirety). The cells are incubated overnight at 4° C. and then washed twice with PBS. Cells are then incubated with a 1:400 dilution of Oregon green 488-conjugated secondary goat anti-rabbit antibody (Invitrogen, Carlsbad, California) for 2 hours at room temperature. Coverslips are mounted using Vectrashield DAPI mounting media (Vector Labs, Burlingame, California) and the images are captured at the same fluorescence intensity for each image at 400 or 600 magnification and processed using an Olympus fluorescent microscope and DP controller software (Olympus Corp., Center Valley, Pennsylvania).

10. Statistical Analysis

All statistical analysis regarding percent proliferating cells as measured by MTT assay and Annexin-FITC assay are performed using JMP Pro (v. 11.2.1; SAS Institute Inc., Cary, North Carolina). The residuals of the statistical model are evaluated for normality and found to be not normally distributed in most analyses. Therefore, non-parametric Kruskal-Wallis testing is used to compare differences in percent proliferating cells for every treatment dose used within each cell line across experiments. Comparisons between each treatment group and vehicle control group are carried out using the Steel method adjusting alpha risk for multiple comparisons.

For the outcome of percent viability determined by the trypan blue exclusion assay residuals of the statistical model are found to be normally distributed, and therefore analyzed using analysis of variance with Dunnett's method for comparison to vehicle control, controlling for multiple comparisons. Differences are considered statistically significant at P<0.05 for all statistical testing.

### Results

1. 48 hours MTT Assays CBD, CBDA and Whole Hemp Extract

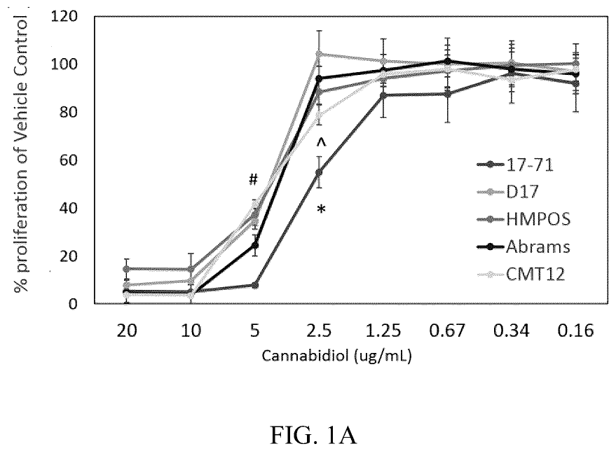

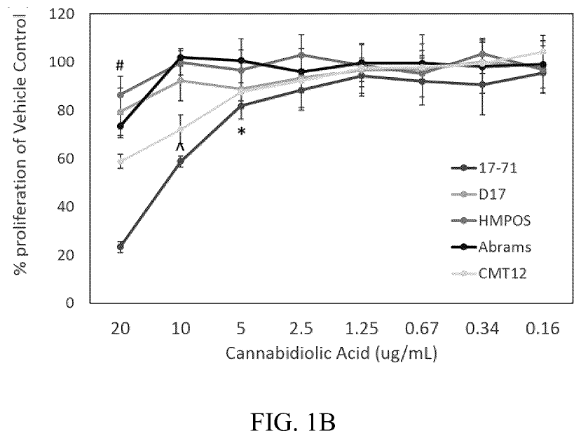

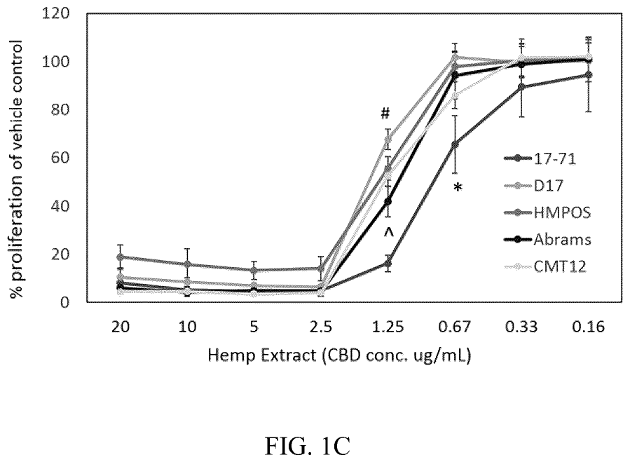

When examining the Probit analysis of CBD on the 5 cell lines it is found that the IC50 for 17-71 cells is 2.5 μg/mL and the concentrations that caused significantly diminished proliferation are 2.5 μg/mL and above. The CMT12 cell line exhibits a similar profile with significantly diminished proliferation at 2.5 μg/mL and above with a slightly higher probit IC50 of 3.5 μg/mL. The Abrams, D17 and HMPOS show similar probit analysis concentrations of 4.1. 4.1 and 3.6 μg/mL respectively, with the concentrations of 5 μg/mL and above being significant for slowing cell proliferation when compared with vehicle control treated cells (FIG. 1A).

Whole hemp extract using concentrations of CBD in the extract of 20 μg/mL and lower in conjunction with other cannabinoids and terpenes shows that lesser CBD concentrations are needed across all cell lines. The hemp extract shows that the probit IC50 is approximately 0.8 μg/mL for the 17-71 cell line, with the first significantly different concentration to slow proliferation being 0.67 μg/mL and higher. CMT12, Abrams, D17, and HMPOS cells probit reveal IC50's of 1.5, 1.3, 1.6 and 1.7 μg/mL, respectively; and these cell lines show significant decreases in proliferation at 1.25 μg/mL and higher when compared with vehicle control treated cells (FIG. 1B).

Considering the hemp extract used has a significant proportion of CBDA, 48 hours MTT assays are performed with CBDA. CBDA treatment of 17-71 cells exhibits an IC50 of 15.1 μg/mL, with concentrations of 5 μg/mL and above showing significant slowing of cell proliferation. The CMT12 cell lines show significant slowing of proliferation starting at 10 μg/mL and higher, and an IC50 could not be determined since 50% growth inhibition is not achieved in the assay. The three osteosarcoma cell lines, Abrams, D17 and HMPOS show significant growth inhibition at 20 μg/mL, while IC50 calculations could not be calculated because of lack of growth inhibition (FIG. 1C).

The graphs from FIG. 1A-1C depict assay results on 3 different time points performed in duplicate. * Depicts initial concentration that is significantly different from vehicle control treated baseline for 17-71 including any higher concentrations (P<0.05), \textcircled{ }depicts initial point that is significantly different from vehicle control treated baseline for CMT12 including any higher concentrations (P<0.05). #Depicts initial point that is significantly different from vehicle control treated baseline for D17, HMPOS and Abrams including any higher concentrations (P<0.05).

2. Trypan Blue Exclusion Assay for CBD

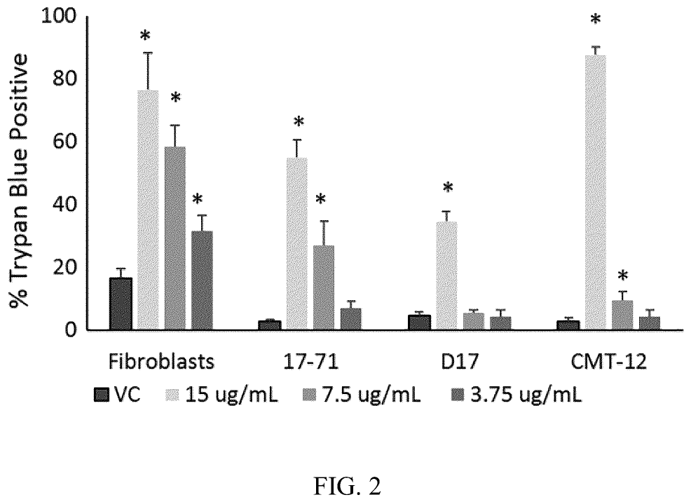

The percent trypan blue positive dermal fibroblasts for methanol vehicle control cells are 16±3% of the cell population. This is significantly higher when treated for 48 hours with 15, 7.5 and 3.75 μg/mL of CBD at 77±12%, 58±7% and 32±5%, respectively (FIG. 2). 17-71 cells show a 3±1% trypan blue positive cell population when treated with methanol as vehicle control. There is a significant increase in trypan blue positive cells at both 15 μg/mL and 7.5 μg/mL at 55%±6% and 27±8%, respectively. 17-71 cells treated with 3.75 μg/mL are no different from vehicle control treated cells at 7±4% positive cells. D17 cells show a 5±1% trypan blue positive cell population when treated with methanol as vehicle control. There is a significant increase in trypan blue positive cells at 15 μg/mL at 35±4%. D17 cells treated with 7.5 and 3.75 μg/mL are not different from vehicle control treated cells at 5±2% and 4±2% positive cells, respectively. CMT12 cells show a 3±1% trypan blue positive cell population when treated with methanol as vehicle control. There is a significant increase in trypan blue positive cells at 15 μg/mL at 87%±8% and 7.5 μg/mL with 9±3%. CMT12 cells treated with 3.75 μg/mL show no difference from vehicle control treated cells at 4±2% (FIG. 2). Each cell line from FIG. 2 is assessed for trypan positive cell death across three experiments with * indicating a significant increase in trypan positive cells as compared with VC cells (P<0.05).

3. Combination Index MTT Dual Treatment Assays with CBD and Chemotherapeutics (Doxorubicin/Vincristine)

When all three cell lines are treated with doxorubicin, the 17-71 lymphoma cell line is most sensitive with inhibitory concentrations from IC20-IC80 between 0.033 and 0.125 μM, while CMT12 and D17 cell lines require higher concentrations to hinder cell proliferation (0.5-2 μM). Regardless of the cell line treated with doxorubicin, when coupled with IC20-IC80 concentrations of CBD, it is evident that at higher concentrations of CBD (10 and 5 μM), there is synergy or additive effects with CI values of less than 1.1 (Table 5). The only universally antagonistic effects (CI values above 1.1) observed across the cell lines are at lower concentrations of CBD (2.5 and 1.25 μg/mL) and lower concentrations of doxorubicin.

TABLE 5
17-71, D17 and CMT12 cell lines CI values under dual treatment with CBD
and doxorubicin (Dox) or CBD and vincristine (Vin) at various concentrations
between the IC80-IC20 concentrations for each drug combination.

| 17-71 | | | | | 17-71 | | | | |
|---|---|---|---|---|---|---|---|---|---|
| CBD μg/mL | 10 | 5 | 2.5 | 1.25 | CBD μg/mL | 10 | 5 | 2.5 | 1.25 |
| Dox 0.125 μM | 0.7 | 0.8 | 1.7 | >2.0 | Vin 1 nM | 1 | 0.8 | 0.5 | 0.4 |
| Dox 0.067 μM | 0.8 | 1 | >2.0 | >2.0 | Vin 0.5 nM | 1 | 0.8 | 0.5 | 0.5 |
| Dox 0.033 μM | 0.7 | 0.8 | >2.0 | >2.0 | Vin 0.25 nM | 1 | 0.9 | 0.9 | 1.1 |
| D17 | | | | | D17 | | | | |
| CBD μg/mL | 10 | 5 | 2.5 | 1.25 | CBD μg/mL | 10 | 5 | 2.5 | 1.25 |
| Dox 2 μM | 1.1 | 0.6 | 0.7 | 0.7 | Vin 6.6 nM | 1.1 | 0.9 | 1.3 | 0.9 |
| Dox 1 μM | 1.1 | 0.6 | 0.7 | 0.9 | Vin 3.3 nM | 0.9 | 0.7 | 1 | 1 |
| Dox 0.5 μM | 1.1 | 0.6 | >2.0 | >2.0 | Vin 1.7 nM | 0.8 | 0.5 | 0.9 | 0.8 |
| CMT12 | | | | | CMT12 | | | | |
| CBD μg/mL | 10 | 5 | 2.5 | 1.25 | CBD μg/mL | 10 | 5 | 2.5 | 1.25 |
| Dox 2 μM | 1 | 0.6 | 0.5 | 0.5 | Vin 6.6 nM | 0.8 | 0.5 | 0.4 | 0.3 |
| Dox 1 μM | 1 | 0.7 | 0.8 | 1.1 | Vin 3.3 nM | 0.7 | 0.5 | 0.5 | 0.3 |
| Dox 0.5 μM | 1 | 0.9 | 1.5 | 1.7 | Vin 1.7 nM | 0.6 | 0.7 | 0.7 | 0.3 |

Notes:
CI values less the 0.9 suggest synergistic interactions, 0.9-1.1 suggest additive interactions and values above 1.1 suggest antagonism between the drug combination. All bolded numbers indicate additive or synergistic interactions while italicized numbers are antagonistic interactions.

When all three cell lines are treated with vincristine, the 17-71 lymphoma cell line again shows to be most sensitive to the treatment with IC20-IC80 values between 0.25 and 1 nM, while CMT12 and D17 cell lines treated with vincristine require higher concentrations to hinder cell proliferation (1.7-6.8 nM). Nearly universally, the treatment of vincristine and CBD showed synergistic or additive effects regardless of the cell line examined with nearly all CI values being 1.1 or lower, suggesting that vincristine and CBD are likely to augment the effects of one another in canine neoplastic cell lines examined (Table 5).

4. Annexin V Apoptosis Assay

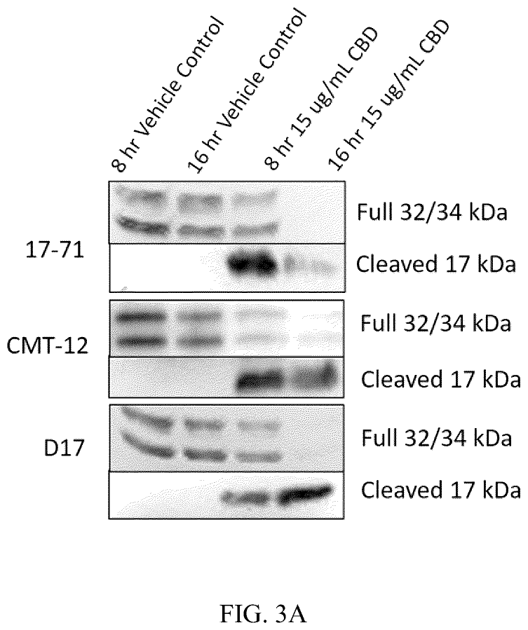

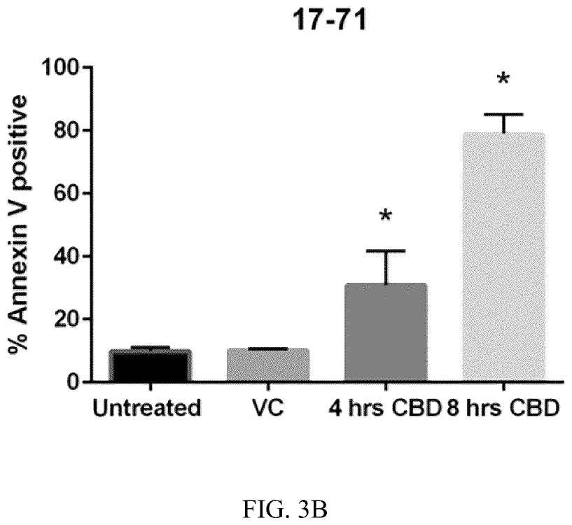

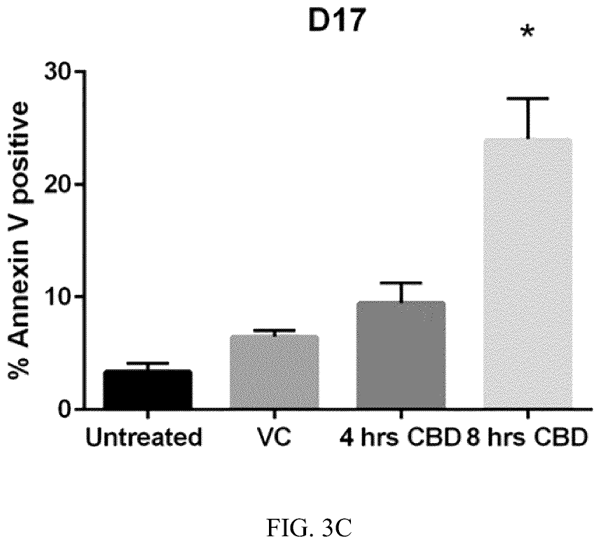

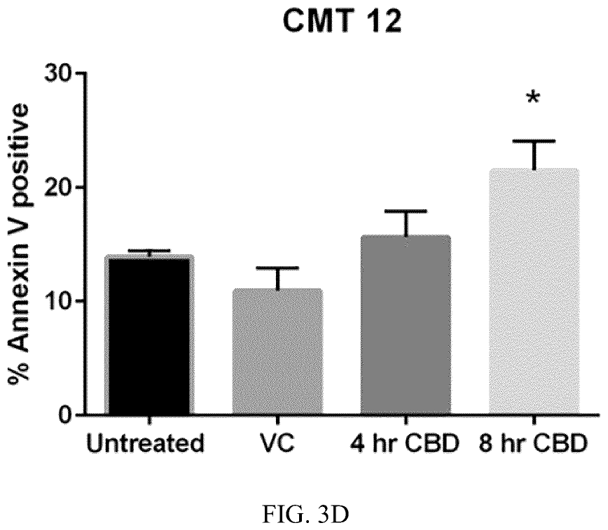

Annexin V apoptosis assays using flow cytometry are performed at 4 and 8 hours after treatment with 15 μg CBD or vehicle control (methanol) for 8 hours. Annexin V staining reveals a mean percentage and SD of 31.0±10.8% and 79.0±6.1% positive staining cells at times 4 and 8 hours respectively in the 17-71 cell line, showing significant increases in positive cells at both 4 and 8 hours when compared with VC cells and untreated cells (10.1±0.4% and 9.9±1.2%, respectively). D17 cells treated with CBD show a significant increase in Annexin V positive cells at only the 8 hour time point (24. 0±3.6%) when compared with VC cells (6.5±0.6%) and untreated cells (3.3±1.0%). D17 cells treated for 4 hours with 15 μg of CBD (9.4±1.8%) are higher that VC or untreated cells, but this increase is not significant. Similarly, the CMT12 cell line shows a significant increase in positive cells at the 8 hour time point with 21.5±2.5% of the cells staining for Annexin V. In the CMT12 cell line, there are also increases in Annexin V stained cells (15.7±2.2%) at 4 hours, but they are not significantly increased from VC treated cells (10.9±2.0%) or untreated (13.9+0.6%) when using nonparametric conservative statistical testing (FIGS. 3B, 3C, and 3D). FIGS. 3B, 3C and 3D are bar charts showing the apoptosis of neoplastic cell lines 17-71, D17 and CMT12 after treatment with 15 μg/mL of CBD. In these figures, the Annexin V apoptosis assay represents the relative percent of Annexin V positive cells identified via flow cytometry. In these figures, the results from the baseline vehicle control treated cells (VC), untreated cells and treated cells (4 and 8 hours of 15 μg/mL CBD treatment) are compared, and “*” indicates a significant increase percentage from VC treatment (P<0.05).

5. Time Course Western Blot Analysis for Pathway Implications

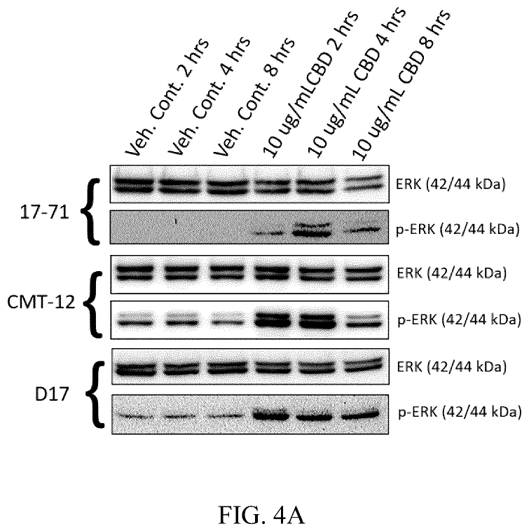

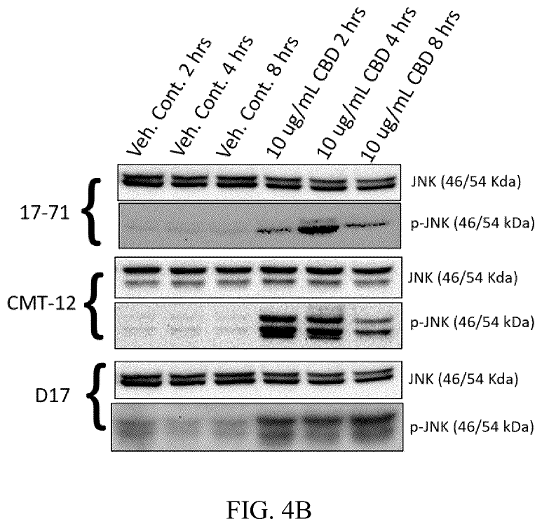

After treatment of cells with methanol vehicle control or 10 μg/mL of CBD for 2, 4 and 8 hours, numerous immunoblots are performed. The immunoblots performed using AKT and phosphorylated-AKT or mTOR and phosphorylated-mTOR show no depression or increased intensity of the signal over time regardless of treatments over two different time course analyses experiments. The lack of changes suggests there is no alteration in the PI3Kinase-AKT-mTOR pathway as it relates to induction of autophagy or apoptosis. Assessment of the MAP kinase pathway shows repeatable increases in both ERK and JNK phosphorylation across all three cell lines in the presence of 10 μg/mL of CBD when compared with equal amounts of methanol vehicle control treatment over the 8 hour time-course (FIGS. 4A and 4B). ERK and phosphorylated ERK expression at time 2, 4 and 8 hours compared with methanol vehicle control treated cells showing extensive phosphorylation of 17-71, D17 and CMT12 cell lines at 2, 4 ad 8 hours. The presence of baseline ERK and JNK phosphorylation is not evident in 17-71 cells and both show abundant phosphorylation peaking at 4 hours of treatment with CBD. The ERK baseline phosphorylation is more evident in D17 and CMT12 cells and is rapidly induced peaking at 2-4 hours in these cell lines (FIG. 4A). The JNK phosphorylation status is robust in the CMT12 cells lines after CBD treatment, while the D17 cells show a milder induction of phosphorylated JNK. Overall, the baseline JNK and ERK protein expression across the cell lines does not change substantially regardless of treatment or time. The blots presented are representative of duplicate time course immunoblotting experiments performed.

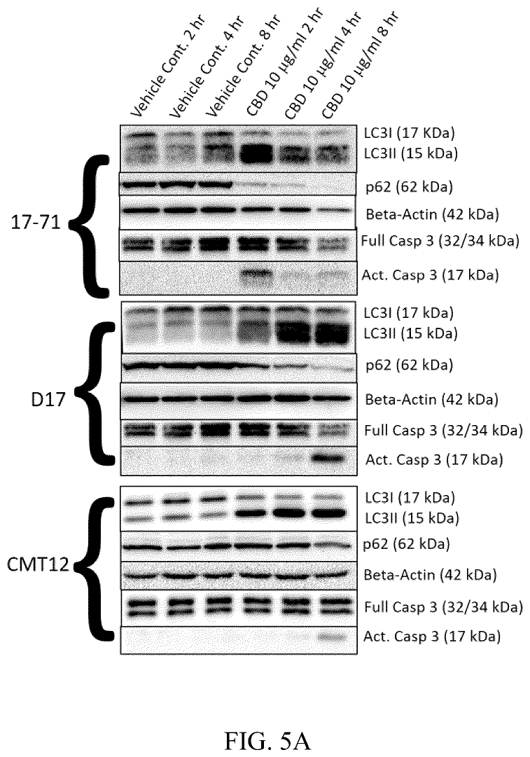

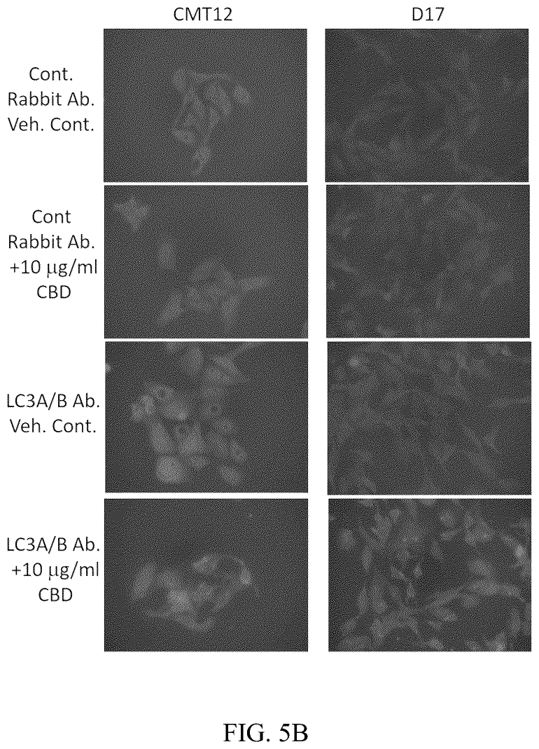

In tandem to these time course analyses, the assessment of LC3 protein is assessed as part of the autophagy response in cells over 8 hours. Universally, in all three cell lines there is an increase in the LC3II proportion of LC3 protein which represents the ethanolamine conjugated form of the protein found in autophagy vesicles or autophagosomes. This increase in LC3II is prominent starting at 2 hours of treatment and persists throughout the 8 hours of treatment which is not observed in vehicle control treated cells. (FIG. 5A). Coupled with the LC3II response, one also observes a marked decrease in western blotting for the autophagy cargo carrier protein p62 which is another marker for autophagy often discussed in the literature (Yoshi S R and Mizushima N, Monitoring and measuring autophagy, Int J Mol Sci, 2017, 18, 1865, which is incorporated herein by reference in its entirety). These observations are also accompanied by an activation of caspase 3 which can also be seen at 2 hours in the 17-71 cells, but is not evident until hour 8 in the CMT12 and D17 CBD treated cells showing autophagy appears to precede the apoptotic response in these cells (FIG. 5A).

6. Immunofluorescence for LC3I/II

Cellular fluorescence imaging is captured at 400 and 600 times magnification for the D17 and CMT12 cell lines, respectively. The use of rabbit polyclonal antibody as a control shows a mild background fluorescence that is minimal with or without 10 μg/mL of CBD treatment (FIG. 5B). FIG. 5B, row 1, shows control Rabbit Antibody immunostaining on vehicle control treated cells. FIG. 5B, row 2, shows control Rabbit Antibody immunostaining on CBD 10 μg/mL treated cells. FIG. 5B, row 3, shows Rabbit LC3A/B antibody immunostaining in vehicle control treated cells showing variable staining of cell cytoplasm. FIG. 5B, row 4, shows rabbit LC3A/B immunostaining in cells treated with 10 μg/mL CBD showing punctate autophagosomes in cytoplasm of cells.

When using the LC3A/B rabbit polyclonal antibody, there is a mild diffuse cytoplasmic staining in methanol vehicle control treated cells regardless of the cell line used. Both the CMT12 and the D17 cells, when treated with 10 μg/mL of CBD immunofluorescence with the LC3A/B antibody, show numerous discrete punctate intensely staining vesicles which are consistent with LC3 protein localization to autophagosomes or autophagolysosomes (FIG. 5B).

### Analysis

An objective of the study is to examine whether CBD and its native acidic form (CBDA) caused similar cytotoxic effect in a select number of common canine cancers, representing mesenchymal, round, and epithelial origins. In accordance with numerous human cell lines studies, the results show that CBD resulted in similar canine cancer cell death uniformly, while CBDA has limited effect at the concentrations used except for a mild effect on the lymphoma cell line at the highest concentrations. These assays are allowed to incubate for 48 hour similar to other natural products studies, which often required longer than 24 hours to induce cytotoxicity.

The results also demonstrate significant reductions in canine cancer cell proliferation when treated with CBD concentrations ranging from 2.5 to 10 μg/mL with similar effects on all 5 cell lines examined. The CBD-rich whole hemp extract used in this study resulted in a significant reduction in cancer cell proliferation at the lowest CBD concentrations, ranging from 0.67 to 10 μg/mL. When using the whole hemp extract, CBD concentrations identical to what is used in the pure CBD experiments are selected. The lethality of CBD in the presence of CBDA and/or other phytocannabinoids and terpenes at lower concentrations can be what potentiated the whole plant extract effects in this study. This synergistic finding is commonly reported in as the “entourage effect,” whereby the mixture of cannabinoids and terpenes can work in concert to produce an augmented effect.

The cell proliferation response of 17-71, CMT12 and D17 cell lines when treated with 10 to 15 μg/mL CBD results in apoptosis, as substantiated by Annexin V staining and cleaved caspase 3 immunoblotting (FIG. 3A), and occurs within 8 hours of treatment. FIG. 3A are images of immunoblottings for cleaved caspase 3 (17 Kda) after 8 and 16 hours of CBD treatment as compared with methanol vehicle control showing cleaved caspase across all cell lines at both time points. In addition, an autophagic response is observed with a proportional phase shift of LC3-I to the phosphatidyl-ethanolamine LC3-II moiety of the protein, p62 degradation and localization of autophagosomes-associated LC3 proteins. In conjunction with this rise in LC3-II, there is a concomitant decrease in the autophagy cargo adapter protein SQSTM1/p62.

The cellular signalling mechanisms assessments reveal no alterations that are pronounced in signalling pathways through AKT or mTOR signalling. However, there is a repeatable and dramatic rise in the phosphorylation of MAP kinase pathways, in particular ERK and JNK signalling, with minimal influence on the p38 phosphorylation and activation. The synergistic and antagonistic results on cancer cell proliferation when canine cancer cells are treated with commonly utilized chemotherapeutics in combination with CBD are significant. The combination of vincristine and CBD consistently potentiated the decrease in cell viability as compared with either treatment alone proving that at lethal to sublethal dosing of both CBD and vincristine there is a prominent synergistic response. Significantly, combination treatment of doxorubicin and CBD results in both synergistic and antagonistic outcomes depending on the specific concentrations of each compound used. Based on the results, as more lethal doses of CBD and doxorubicin are utilized there can be an additive to synergistic response, while at sublethal dosing the effect can be antagonistic. The doses of doxorubicin used in these in-vitro assays is at the higher end of what is observed in serum of dogs during IV infusion when treating the CMT12 and D17 cell lines (1-2 μM) while the 17-71 cell line was far more sensitive to doxorubicin treatment at nearly 1/10^{th} the concentration. Similarly, the 17-71 cell line is more sensitive to vincristine than the D17 and CMT12 cell lines and all of the concentrations used are at 5-20-fold lower than what has been observed to be effective in canine clinical use which is approximately 20 to 40 nM.

### Example 16

Canine Atopy Study

A study was conducted to determine the efficacy of hemp extract on treating atopy in dogs.

The study was a double-blinded, prospective, placebo controlled, randomized study. Client-owned dogs with atopic dermatitis (cAD) were included. Eligible dogs were diagnosed with cAD based on published guidelines. Owner consent was obtained for each case before the study. Parasitic and infectious causes of pruritus were excluded with negative combings, skin scrapings, and cytological examinations. Dogs in the study showed no improvement with a prior two month hydrolyzed or novel protein exclusion diet. For each case, a dermatological examination was performed and lesions, and pruritus were evaluated using validated scoring systems. Only cases of localized and mild to moderate cAD were included. Mild to moderate cAD is defined as those patients with a Pruritus Visual Analog Scale (pVAS) score between 3 and 7 and a Canine Atopic Dermatitis Extent and Severity Index-4 (CADESI-4) score between 10 and 35.

Concurrent administration of cyclosporine (either brand name Atopica or generic microemulsion) are not allowed, and patients who have received this medication in the past two months are excluded. Concurrent administration of Allergen Specific Immunotherapy, injectable or sublingual, is allowed for patients who have received this treatment for at least one year prior to the initial study. Concurrent administration of Oclacitinib, oral glucocorticoids, ketoconazole and other imidazoles is allowed for patients who have received these medications for at least two months prior to the initial study. Patients who have received a Cytopoint injection within three months of the initial study are excluded. No other treatments, concomitant medications, or changes in medications are allowed during the study. No change in the patient's typical bathing routine is allowed during the study. No dietary changes are allowed during the study.

Investigators were blinded to the group assignments. Group A consisted of those patients receiving oral, oil based CBD:CBDA product at a dose of 2 mg/kg twice daily in a capsule form. Group B consisted of those patients receiving a matching placebo oil in capsule form. Dog with concurrent co-morbidities such as kidney failure, hepatic disease, endocrine diseases or other immunological dysfunctions (ITP, I MHA) were excluded. The study involved 29 dogs with cAD (17 in the treatment group and 12 in the control group). Dosage with oral CBD:CBDA product was approximately 2 mg/kg twice daily for twenty-eight days. Owners were asked to administer the drug with a meal.

During the duration of the study, the doses of allowed medications the patient was receiving prior to the study remain unchanged. Additionally, the medication doses were not changed within the 21 days prior to the initiation of the study.

### Procedures

Clients were asked to sign a consent form. On day (0) each dog received an initial consultation by an investigator including a thorough history and physical examination. Treatment was initiated as described above and follow up visits are conducted at weeks 2 and 4 weeks. Changes in owner perception of the extent and severity of atopic dermatitis were evaluated using the pVAS. Changes in the extent and severity of atopic dermatitis were evaluated by the veterinarian based CADESI-4. Benchmarks separating normal/remission from mild, moderate and severe cAD using CADESI-4 are 10, 35 and 60, respectively, as suggested by a validating study. Prior to the onset of the investigation, all participating investigators were trained in how to evaluate and interpret the CADESI-4, which includes evaluating clinical case examples. 1 cc of serum from visits 0 and 4 was stored at −20° C. for future cytokine analysis and for serum CBD testing.

### Analyses

Current power analysis revealed that if there is a 2.5 standard deviation and a 2.0 change in VAS scoring by owners, that the necessary population to find a statistical significance is approximately 13 patients. To ensure adequate enrollment and completion of the trial, 18 dogs were enrolled based on a random number generator allocating up to 36 patients into groups A or B. After data collection and cleaning, all scores and blood parameters were assessed using a mixed model analysis of variance including the effects of time, treatment, concurrent medications, and the random effect of dog with an alpha set at 0.05 or less as significant. If differences were found, Tukey's post hoc analysis was performed to assess the differences between time points.

### Results

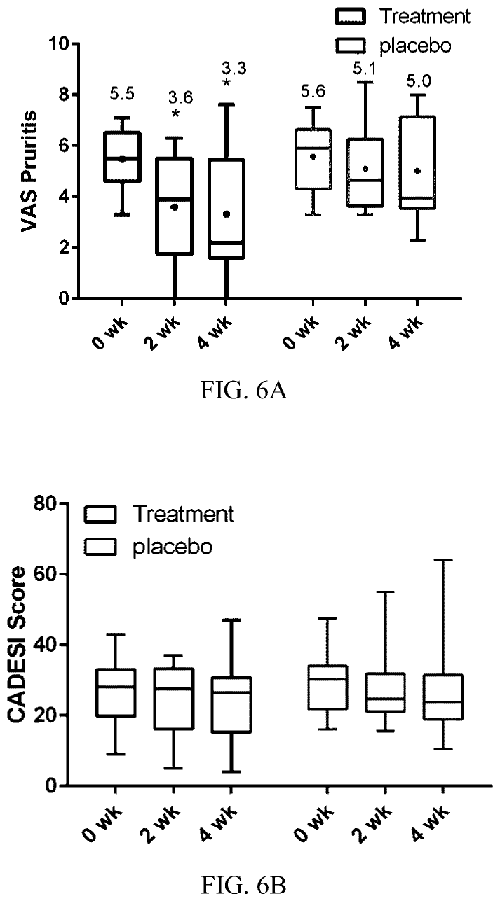

FIG. 6A shows VAS scores for the treatment group and control group. FIG. 6B shows CADESI scores for the treatment group and control group at zero and 4 weeks. Significant differences are marked with asterisks at zero and 4 weeks.

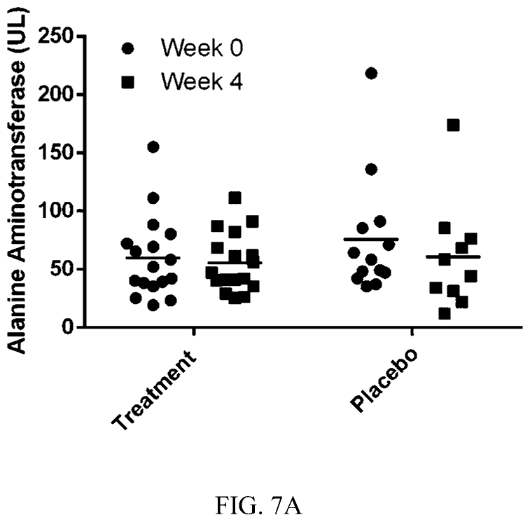

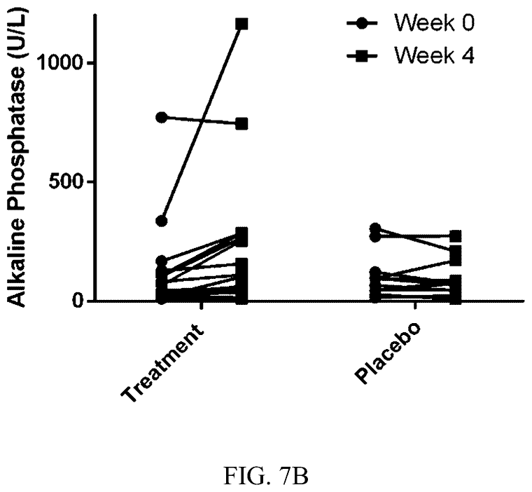

FIG. 7A shows alkaline aminotransferase levels in dogs treated with either the CBD treatment or the placebo. Values are shown for both zero and 4 weeks. FIG. 7B shows alkaline phosphatase levels in dogs treated with either the CBD treatment or the placebo. Values are shown for both zero and 4 weeks.

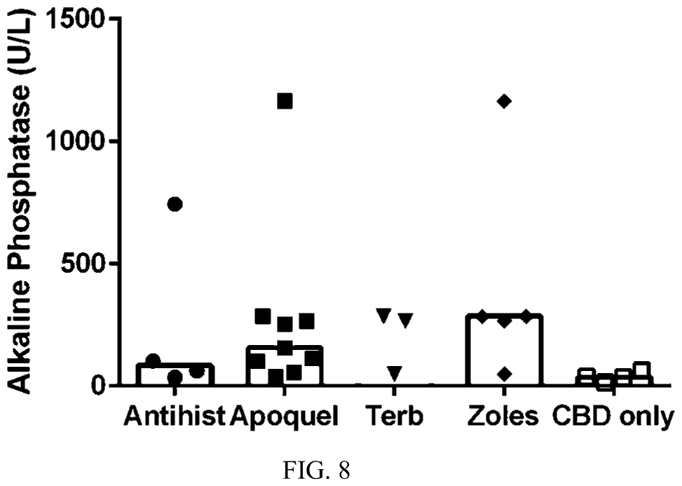

FIG. 8 shows alkaline phosphatase levels in dogs when the CBD treatment was combined with various other drugs as indicated, or when the CBD treatment was administered without other drugs (right-most column).

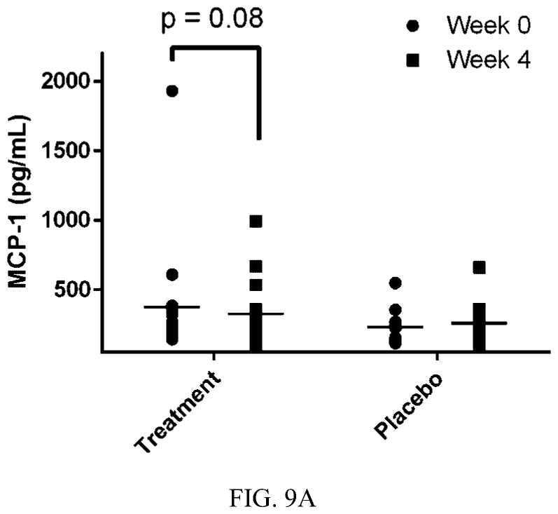

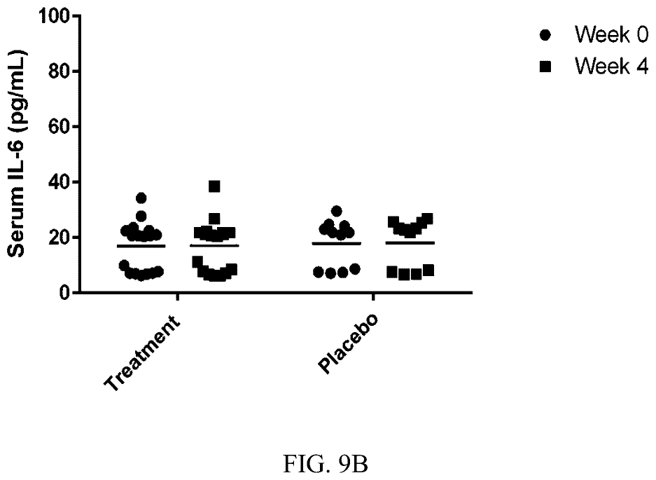

FIG. 9A shows serum levels of monocyte chemoattractant protein-1 (MCP-1) for study participants at zero and 4 weeks. FIG. 9B shows serum levels of IL-6 for study participants at zero and 4 weeks.

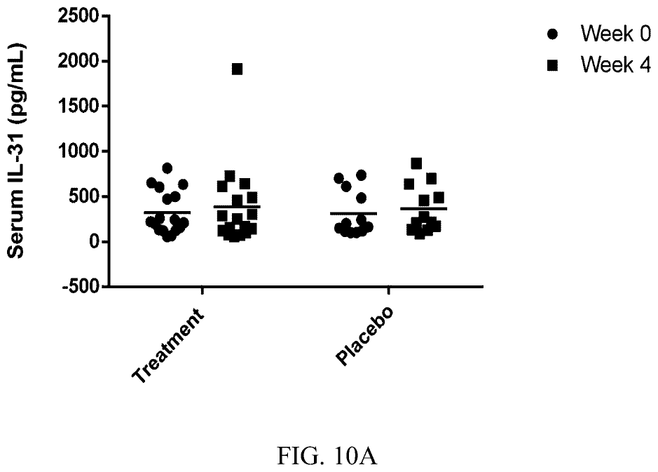

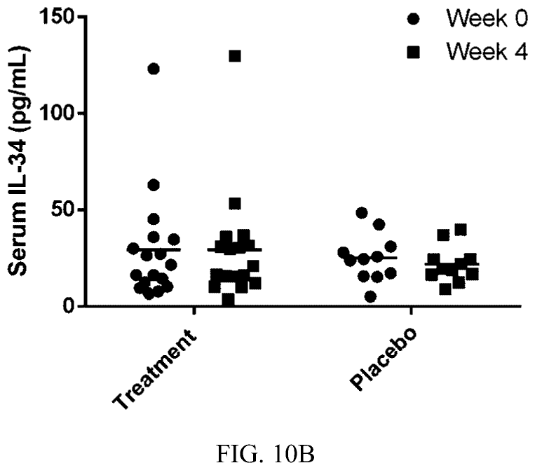

FIG. 10A shows serum levels of IL-31 for study participants at zero and 4 weeks. FIG. 10B shows serum levels of IL-34 for study participants at zero and 4 weeks.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

1. A method for treating recurrent diarrhea disease in a veterinary subject in need thereof, the method comprising administering to the subject a therapeutically effective amount of hemp extract.

2. The method of claim 1, wherein the veterinary subject is a primate.

3. The method of claim 2, wherein the primate is a non-human primate.

4. The method of claim 3, therein the non-human primate is a macaque.

5. The method of claim 1, wherein the recurrent diarrhea disease comprises idiopathic diarrhea.

6. The method of claim 1, wherein the hemp extract is administered orally.

7. The method of claim 1, wherein the hemp extract is administered in the form of a chew, a marshmallow, a gummy, or via syringe.

8. The method of claim 1, wherein the hemp extract is administered at a dosage of about 2 mg/kg, about 4 mg/kg, or about 8 mg/kg.

9. The method of claim 1, wherein the hemp extract is administered until the veterinary subject has a stool score of ≤2 for 3 days or for 21 days.

10. The method of claim 1, wherein the hemp extract is administered once a day, twice a day, three times a day, or four times a day.

11. A method for treating inflammation, noise aversion, pyoderma, atopy, lameness, or arthritis or promoting wound healing in a veterinary subject in need thereof, the method comprising administering to the subject a therapeutically effective amount of hemp extract.

12. The method of claim 11, wherein the veterinary subject is a dog, a horse, or a rabbit.

13. The method of claim 11, wherein the hemp extract comprises CBD, CBDA, CBG, CBGA, or a combination thereof.

14. The method of claim 11, wherein the hemp extract modulates neutrophil function, generation of reactive oxygen species, phagocytosis, eicosanoid concentrations, chemotaxis, cytokine production, and/or fibroblast response.

15. (canceled)

16. (canceled)

17. The method of claim 11, wherein the noise aversion is a phobia or wherein the noise aversion is to fireworks or thunderstorms.

18-30. (canceled)

31. The method of claim 11, wherein the hemp extract is administered topically or orally.

32-51. (canceled)

52. A method for treating a neural condition in a subject in need thereof, comprising administering to the subject a therapeutically effective amount of hemp extract.

53. The method of claim 52, wherein the subject is a human or a veterinary subject.

54. (canceled)

55. The method of claim 52, wherein the neural condition comprises a traumatic neural injury or a degenerative neural disease.

56. The method of claim 52, wherein the hemp extract comprises THCA.

57-63. (canceled)

64. The method claim 1, wherein the hemp extract comprises:
cannabidiol; and
cannabidiolic acid;
wherein the ratio of cannabidiol to cannabidiolic acid is about 0.1:1 to about 1:0.1.

65. The method of claim 64, wherein the hemp extract further comprises:
cannabigerolic acid;
Δ9-tetrahydrocannabinol; and
cannabichromene.

66-80. (canceled)

81. The method of claim 1, wherein the hemp extract is formulated in a carrier.

82. The method of claim 81, wherein the carrier is selected from the group consisting of hemp seed oil, linseed oil, olive oil, fish oil, salmon oil, coconut oil, catnip oil, sesame oil, MCT oil, and grapeseed oil.

83-85. (canceled)

86. The method of claim 1, wherein the hemp extract comprises lecithin.

87. The method of claim 86, wherein the lecithin is sunflower lecithin.

88-106. (canceled)

107. The method of claim 1 wherein the hemp extract is administered in a dosage form comprising one or more pharmaceutically acceptable additives, flavoring agents, surfactants, and adjuvants.

108-129. (canceled)

130. The method of claim 107, wherein the dosage form is formulated as a chew for oral administration.

131-161. (canceled)
